(19) United States
(12) Patent Application Publication
(10) Pub. No.: US 2025/0352490 A1
(43) Pub. Date: Nov. 20, 2025

(54) P-SELECTIN TARGETED NANOPARTICLES AND USES THEREOF

(71) Applicant: Ramot at Tel-Aviv University Ltd., Tel-Aviv (IL)

(72) Inventors: Ronit SATCHI-FAINARO, Tel-Aviv (IL); Shani MICHAEL, Tel-Aviv (IL); Pradip DEY, Tel-Aviv (IL)

(73) Assignee: Ramot at Tel-Aviv University Ltd., Tel-Aviv (IL)

(21) Appl. No.: 19/287,868

(22) Filed: Aug. 1, 2025

Related U.S. Application Data
(63) Continuation of application No. PCT/IL2024/050131, filed on Feb. 2, 2024.
(60) Provisional application No. 63/442,780, filed on Feb. 2, 2023.

(51) Int. Cl. A61K 9/51; A61K 31/5025; A61K 47/69; A61P 35/00

(52) U.S. Cl. CPC A61K 9/5153; A61K 31/5025; A61K 47/6937; A61P 35/00

(57) ABSTRACT

Particles made of a polymeric matrix having associated therewith a therapeutically active agent usable in treating a medical condition associated with an overexpression of P-selectin in a subject in need thereof and featuring a P-selectin selective targeting moiety represented by Formula I as defined and described in the specification and claims, compositions comprises these particles and uses thereof, are provided.

### RELATED APPLICATIONS

This application is a Continuation of PCT Patent Application No. PCT/IL2024/050131 having International filing date of Feb. 2, 2024, which claims the benefit of priority under 35 USC § 119(e) of U.S. Provisional Patent Application No. 63/442,780 filed on Feb. 2, 2023. The contents of the above applications are all incorporated by reference as if fully set forth herein in their entirety.

### FIELD AND BACKGROUND OF THE INVENTION

The present invention, in some embodiments thereof, relates to therapy and, more particularly, but not exclusively, to targeted polymeric nanoparticles and uses thereof as delivery vehicles of therapeutically active agents.

In the past decade, promising targeted therapies were developed to treat melanoma. Yet, 70% of the patients undergo relapse during the first 3-5 years due to the development of acquired resistance. Targeted therapies have also been studied to treat other cancer types, particularly brain cancers and brain metastases and breast cancers.

One of the most common causes for melanoma progression is BRAF mutations that occur in 50-70% of melanoma patients and lead to a constitutive activation, independent of extracellular factors, of the mitogen-activated protein kinase (MAPK) pathway, which results in cell proliferation and survival. BRAF mutations were found in several cancer types, other than melanoma, including glioblastoma, colon, anaplastic thyroid, non-small cell lung cancer [see, for example, Wen, P. Y., et al. Lancet Oncol (2021)]. BRAF and MEK inhibitors are considered the standard of care (SoC) for BRAF-mutant melanoma patients, alongside immunotherapy with anti-PD-1 (Nivolumab or Pembrolizumab) and anti-CTLA-4 (Ipilimumab) antibodies.

To overcome resistance, a combination treatment of MEKi with BRAFi was examined. The combination of DBF and TRM improved the overall survival (OS) and progression-free survival (PFS) of melanoma patients compared to DBF alone (25.1 versus 18.7 months, and 11 versus 8.8 months, respectively), and therefore the combination is the first line of treatment for melanoma patients with BRAF V600E or V600K mutations with (1) unresectable or metastatic disease or (2) as adjuvant therapy for patients with lymph nodes involvement. The combined treatment reduced the incidence of cutaneous squamous cell carcinoma associated with DBF [Flaherty, K. T., et al. New England Journal of Medicine 367, 1694-1703 (2012); Long, G. V., et al. New England Journal of Medicine 371, 1877-1888 (2014); Davies, M. A., et al. The Lancet Oncology 18, 863-873 (2017); and Long, G. V., et al. Lancet 386, 444-451 (2015)].

Nevertheless, adverse events cause treatment discontinuation in 18% of the patients, and the 5 years PFS rates were only 19% with a median PFS duration of 11.1 months [Robert, C., et al. The New England journal of medicine 381, 626-636 (2019)].

The main factor that besets the response duration is acquired resistance, which originates predominantly in reactivation of the MAPK pathway through BRAF ultra-amplification or concurrent mutations in MEK1/MEK2 or RAS.

Additionally, DBF and TRM demonstrate limited brain penetration (steady-state brain to plasma concentration ratio was 0.019±0.02 for DBF and 0.03±0.01 for TRM). Due to the high tendency of melanoma to develop brain metastases, it may render the brain as a sanctuary for tumor cells. Thus, the incidence rate of de novo brain metastases (about 40%) did not change with the introduction of the new targeted therapies or immunotherapies, which means that brain metastases remain a therapeutic challenge.

Hence, there is an unmet medical need to facilitate the penetration of the drugs into the brain, especially in the early stages of the micrometastases, where the tumor cells reside behind an intact blood-brain barrier (BBB).

Breast cancer (BC) is the most frequently diagnosed cancer and the second most common cause of cancer mortality in women worldwide [Siegel et al., Cancer statistics, 2020. CA: A Cancer Journal for Clinicians 70, 7-30 (2020)]. Approximately 15% of all BC are triple negative (TNBC), among them, 30% are BRCA1- or BRCA2-mutated [S. De Talhouet et al., Sci Rep 10, 19248 (2020)]. These tumors are highly aggressive and invasive.

Recently, inhibition of poly(ADP-ribose)polymerase-1 (PARP1), a DNA repair enzyme, was shown to induce “synthetic lethality” in BRCA-mutated cancer cells prolonging PFS (Progression Free Survival) [Turkand Wisinski, Cancer 124, 2498-2498 (2018); Huang et al. Nature Reviews Drug Discovery 19, 23-38 (2020)]. This led to the FDA approval of PARP inhibitors (PARPi) for the treatment of BRCA-mutated BC. Despite their promise, resistance mechanisms to PARPi often develop affecting drug availability, (de)PARylation enzymes, restoration of Homologous Recombination (HR) or restoration of replication fork stability. Moreover, PARPi have been shown to have an impact on cancer-associated immunity, and their combination with immune checkpoint therapy (ICT) has been explored in clinical trials [H. Sato et al., Nature Communications 8, 1751-1751 (2017); S. Jiao et al., Clinical Cancer Research 23, 3711-3720 (2017); E. J. Lampert et al., Clin Cancer Res 26, 4268-4279 (2020); A. S. Zimmer et al., J Immunother Cancer 7, 197 (2019)].

To date, several PARP inhibitors have been approved by the FDA for the treatment of various cancer types, including, for example, ovarian cancer and breast cancer, including BRCA-mutated BC. Despite the potential of these therapeutic agents, BRCA-mutated BC tumors have been proved to acquire resistance to PARPi therapy through diverse mechanisms [L. J. Barber et al., J Pathol 229, 422-429 (2013); B. Norquist et al., J Clin Oncol 29, 3008-3015 (2011); C. Cruz et al. Ann Oncol 29, 1203-1210 (2018); W. Sakai et al., Cancer Res 69, 6381-6386 (2009)]. In addition, up-regulation of P-glycoprotein expression through PARPi therapy has been proved to increase drug efflux and consequently to reduce PARPi concentration in the cytoplasm [S. Rottenberg et al., Proc Natl Acad Sci USA 105, 17079-17084 (2008)]. PARPi were shown to promote the loss of DNA-repair proteins such as P53-binding protein (P53BP1) and REV7 or on the contrary, increase activity of MET/HGFR and PI3K/AKT signalling cascades that might reduce the affinity of PARPi to PARP protein [J. E. Jaspers et al., Cancer Discov 3, 68-81 (2013); A. Tapodi et al., J Biol Chem 280, 35767-35775 (2005); G. Xu et al., Nature 521, 541-544 (2015)]. There is also evidence of restoration of BRCA1/2 mutation during PARPi treatments [B. Norquist et al., J Clin Oncol 29, 3008-3015 (2011); W. Sakai et al., Cancer Res 69, 6381-6386 (2009)].

Thus, combined therapies were explored to allow reducing the dose of PARPi and to be the solution to postpone the occurrence of resistance to this drug by increasing the percent of injected dose that reaches the tumor thus, leading to a more powerful anti-tumor therapy [Miller et al., J Gynecol Oncol 33, e44 (2022)]. Accumulating evidence has suggested that conventional and targeted anticancer therapies like PARPi might trigger tumor-immune responses by recruiting cells that support tumor growth [Galluzzi et al. Cancer Cell 28, 690-714 (2015); Lee and Konstantopoulos, Ther Adv Med Oncol 12, 1758835920944116 (2020); Jijon et al., American Journal of Physiology—Gastrointestinal and Liver Physiology 279, G641-G651 (2000); Haddad et al., British Journal of Pharmacology 149, 23-30 (2006); Laudisi et al., Endocrine, Metabolic & Immune Disorders—Drug Targets 11, 326-333 (2011)]. Additionally, PARPi affect dendritic cell (DC) maturation, as they were shown to reduce the expression of DC activation markers (CD86 and CD83) as well as the production of pro-inflammatory cytokines (IL-12 and IL-10). PARPi can also protect CD8+ lymphocytes from radical oxygen-induced apoptosis [Aldinucci et al., The Journal of Immunology 179, 305-312 (2007); Thorén et al., The Journal of Immunology 176, 7301-7307 (2006)]. Additionally, PARPi treatment was shown to directly upregulate PD-L1 expression and enhance cancer-associated immunosuppression both in vitro and in vivo [Sato et al., Nature Communications 8, 1751-1751 (2017); S. Jiao et al., Clinical Cancer Research 23, 3711-3720 (2017)]. BRCA1-mutated BC have been also related to high basal expression of PD-L1 and high abundance of tumor-infiltrating immune cells [Wen and Leong, PLOS ONE 14, e0215381-e0215381 (2019)].

Currently, several clinical trials combining PARPi and anti-PD-L1 are being studied for BRCA-mutated cancers [L. Musacchio et al., ESMO Open 7, 100536 (2022)]. Although 3 antibodies targeting PD-L1 have been approved recently by the FDA for the treatment of several cancer types, such as Atezolizumab (March 2019) for TNBC, Avelumab (May 2019) for renal cell carcinoma (RCC) and Durvalumab (November 2022) for urothelial carcinoma, these therapeutic agents are administered intravenously, and exhibit side-effects. In addition, antibodies present other disadvantages such as high production costs, poor tumor accumulation as well as poor uptake and poor tissue penetration [Chames et al. British journal of pharmacology 157, 220-233 (2009); Kaplon et al. MAbs 15, 2153410 (2023); Y. Y. Syed, Erratum to: Durvalumab: First Global Approval. Drugs 77, 1817 (2017)].

Small molecules have at least the following advantages over antibodies: (i) higher oral bioavailability, (ii) better diffusion within the tumor microenvironment, (iii) enhanced targeting of intracellular proteins since they easily cross the cellular membrane, and (iv) ability to escape from tumor-associated macrophage-mediated resistance. Therefore, recent efforts have been focused on the development of small-molecule immune checkpoint inhibitors [Acúrcio et al., (2022), supra; Adams et al. Nat Rev Drug Discov 14, 603-622 (2015); Zhan et al., Drug Discov Today 21, 1027-1036 (2016): Arlauckas et al. Sci Transl Med 9 (2017)].

However, delivery of small molecules also imposes several challenges, including low drug solubility, rapid clearance, poor intracellular penetration, and endosomal release [Zhong et al., Signal Transduct Target Ther 6, 201 (2021)].

WO 2017/145164 describes the design, preparation, drug delivery, and properties of conjugates in which BRAF and/or MEK inhibitors (modified dabrafenib and selumetinib, respectively) are covalently linked to poly(a, L-glutamic acid) (PGA) or loaded into poly(lactic-co-glycolic acid) (PLGA) nanoparticles. The nanoconjugate enhanced the solubility and stability of the drugs and facilitated selective drug release by cathepsins at the tumor site. The combined treatment led to an antitumor effect in mice.

WO 2022/175955, which is incorporated by reference as if fully set forth herein, describes the design and preparation of small molecules which are usable as modulators of PD-1/PD-L1 interaction and/or as enhancers of T-cell function.

P-selectin (SELP) is a cell adhesion molecule responsible for leukocyte recruitment and platelet binding, which is expressed constitutively in endothelial cells. Upon endothelial activation with ionizing radiation, P-selectin translocates to the cell membrane [Hallahan et al. Cancer Res 58, 5216-5220 (1998); Bonfanti et al. Blood 73, 1109-1112 (1989)]. Elevated P-selectin expression has been found in the vasculature of human colon, breast, kidney and other cancers [Hanley, W. D., et al. FASEB J 20, 337-339 (2006); Shamay, Y., et al. Science translational medicine 8, 345ra387 (2016); Ferber, S., et al. Elife 6 (2017); and Yeini, E., et al. Nat Commun 12, 1912 (2021)]. P-selectin has also been reported to promote metastasis by arresting circulating tumor cells at the pre-metastatic niche and enabling the tumor cells to extravasate through the activated blood vessels and facilitate colonization [Lorenzon, P., et al. J Cell Biol 142, 1381-1391 (1998); Hoos, A., et al. Cancer Res 74, 695-704 (2014); Natoni, A., et al. Front Oncol 6, 93 (2016); and Laubli, H. & Borsig, L. Semin Cancer Biol 20, 169-177 (2010)]. SELP is also known to be expressed on activated endothelial cells and platelets at inflammation sites [Kansas, G.S. Blood 88, 3259-3287 (1996)].

Additional background art includes U.S. Pat. No. 9,737,614; Shamay et al., Sci Transl Med. 2016 June 29; 8(345): 345ra87; Tylawsky et al., Nature Materials, Volume 22, March 2023, pp. 391-399; Danhier, F., et al. J Control Release 161, 505-522 (2012); Shamay, Y., et al. Science translational medicine 8, 345ra387-345ra387 (2016); Solhi, L., et al. Molecular Systems Design & Engineering 5, 1671-1678 (2020); Dernedde, J., et al. Proc Natl Acad Sci USA 107, 19679-19684 (2010); Weinhart, M., et al. Macromol Biosci 11, 1088-1098 (2011); Kratz, F. & Warnecke, A. J Control Release 164, 221-235 (2012); Eldar-Boock, et al. Curr Opin Biotechnol 24, 682-689 (2013); Shi, D., et al. Adv Drug Deliv Rev 180, 114079 (2021); Abstiens et al. ACS Appl Mater Interfaces 11, 1311-1320 (2019); Dernedde, J., et al. Proc Natl Acad Sci USA 107, 19679-19684 (2010); and Weinhart, M., et al. Macromol Biosci 11, 1088-1098 (2011).

### SUMMARY OF THE INVENTION

According to an aspect of some embodiments of the present invention there is provided a composition comprising a plurality of particles, wherein in at least a portion of the particles, each particle comprises a polymeric matrix having associated therewith at least one therapeutically active agent usable in treating a medical condition associated with an overexpression of P-selectin in a subject in need thereof, wherein in at least a portion of the particles which comprise the polymeric matrix, the polymeric matrix has attached to a surface thereof a P-selectin selective targeting moiety represented by Formula I:

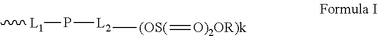

or a pharmaceutically acceptable salt thereof, wherein: R is hydrogen or alkyl; the curved line represents an attachment point to the polymeric matrix; P is an amphiphilic polymeric or oligomeric moiety; L₁ and L₂ are each independently a linking moiety or absent; and k is an integer ranging from 1 to 10, or from 1 to 6, or from 1 to 3, wherein when k is greater than 1, L₂ is or comprises a branching unit.

According to some of any of the embodiments described herein, P is or comprises a poly(alkylene glycol) moiety.

According to some of any of the embodiments described herein, an average molecular weight of the polymeric moiety ranges from about 100 to about 10,000, or from about 500 to about 5,000, or from about 1,000 to about 5,000, or from about 1,000 to about 3,000 grams/mol.

According to some of any of the embodiments described herein, L₁ and L₂ are each independently selected from an alkyl, an aminoalkyl, a hydroxyalkyl, a thioalkyl, an ether, a thioether, —O—, —S—, an amine, —C(═O)—, —C(═S)—, an amide, a carbamate, a carboxylate, a thiocarboxylate, a thiocarbamate, a thioamide, sulfonate, sulfoxide, phosphonate, sulfonamide, urea, thiourea, hydrazine, hydrazide, a hydrocarbon substituted or interrupted by any of the foregoing, and any combination thereof.

According to some of any of the embodiments described herein, L₁ is or comprises an amine or an aminoalkyl.

According to some of any of the embodiments described herein, L₂ is or comprises an amine or an aminoalkyl.

According to some of any of the embodiments described herein, k is 1.

According to some of any of the embodiments described herein, L₂ is or comprises a hydrocarbon interrupted by one or more of —O—, —S—, an amine, —C(═O)—, —C(═S)—, an amide, a carbamate, a carboxylate, a thiocarboxylate, a thiocarbamate, and a thioamide.

According to some of any of the embodiments described herein, the targeting moiety is represented by:

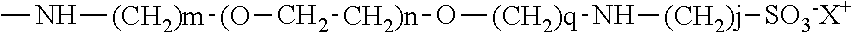

wherein: n is an integer of at least 10, or at least 20; each of m, q and j is independently 0, 1, 2, 3 or 4; and X⁺ is a monocation.

According to some of any of the embodiments described herein, k is greater than 1, and L₂ is or comprises the branching unit.

According to some of any of the embodiments described herein, the branching unit is derived from glycerol.

According to some of any of the embodiments described herein, k is 2 and the targeting moiety is represented by:

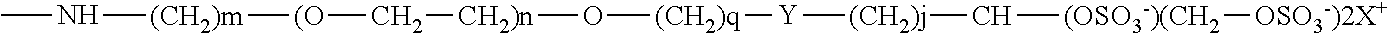

wherein: n is an integer of at least 10, or at least 20; each of m, q and j is independently 0, 1, 2, 3 or 4; X⁺ is a monocation; and Y is selected from —O—, —S—, an amine, —C(═O)—, —C(═S)—, an amide, a carbamate, a carboxylate, a thiocarboxylate, a thiocarbamate, and a thioamide.

According to some of any of the embodiments described herein, Y is a carbamate.

According to some of any of the embodiments described herein, Y is —S—.

According to some of any of the embodiments described herein, the medical condition is a SELP-expressing cancer.

According to some of any of the embodiments described herein, the medical condition is selected from melanoma, primary brain cancer (e.g., glioblastoma), brain metastases (originating from melanoma, lung cancer, breast cancer and colorectal cancer), colon cancer, pancreatic cancer, non-small cell lung cancer, ovarian carcinoma, head and neck squamous cell carcinoma, breast cancer, kidney cancer (e.g., renal cell carcinoma), pediatric glioma (e.g., pediatric low-grade glioma, DIPG, medulloblastoma, pilocytic astrocytoma followed by ganglioglioma, papillary craniopharyngioma), metastases thereof, and inflammation.

According to some of any of the embodiments described herein, the at least one therapeutically active agent is selected from a MEK inhibitor (e.g., pimasertib, binimetinib, cobimetinib, refametinib, selumetinib, trametinib, mirdametinib (PD325901), PD318088, PD334581, PD98059, PD184352 (CI-1040), AZD6244 (ARRY-142886). RDEA119, MEK162 (ARRY-438162)); a BRAF inhibitor (e.g., encorafenib (LGX818), dabrafenib, vemurafenib, sorafenib, GDC-0879, N-[3-(5-chloro-1H-pyrrolo[2,3-b]pyridin-3-ylcarbonyl)-2,4-difluorophenyl]propane-1-sulfonamide (PLX4720), (3R)—N-(3-[[5-(2-cyclopropylpyrimidin-5-yl)-1H-pyrrolo[2,3-b]pyridin-3-yl]carbonyl]-2,4-difluorophenyl)-3-fluoropyrrolidine-1-sulfonamide (PLX8394)); an EGFR inhibitor (e.g., cetuximab, panitumumab, osimertinib (merelectinib), erlotinib, gefitinib, necitumumab, neratinib, lapatinib, vandetanib, brigatinib); a poly ADP ribose polymerase (PARP) inhibitor (e.g., talazoparib, olaparib, rucaparib, niraparib, veliparib, pamiparib, iniparib, CEP9722, E7016); an inhibitor of HER2 and/or HER3 (e.g., lapatinib, trastuzumab. AC-480, erlotinib, gefitinib, afatinib, neratinib, CDX-3379, U-31402, HMBD-001, MCLA-128, KBP-5209, poziotinib, varlitinib, FCN-411, elgemtumab, sirotinib); a SHP2 inhibitor (e.g., 6-(4-amino-4-methylpiperidin-1-yl)-3-(2,3-dichlorophenyl)pyrazin-2-amine (SHP099), [3-[(3S,4S)-4-amino-3-methyl-2-oxa-8-azaspiro[4.5]decan-8-yl]-6-(2,3-dichlorophenyl)-5-methylpyrazin-2-yl]methanol (RMC-4550) RMC-4630, TNO155); an Axl inhibitor (e.g., R428, BGB324, crizotinib, bosutinib, cabozantinib, sunitinib, foretinib, merestinib, glesatinib), a PI3K inhibitor (e.g., buparlisib (BKM120), alpelisib (BYL719), samotolisib (LY3023414), 8-[(1R)-1-[(3,5-difluorophenyl)amino]ethyl]-N,N-dimethyl-2-(morpholin-4-yl)-4-oxo-4H-chromene-6-carboxamide (AZD8186), tenalisib (RP6530), voxtalisib hydrochloride (SAR-245409), gedatolisib (PF-05212384), panulisib (P-7170), taselisib (GDC-0032), trans-2-amino-8-[4-(2-hydroxyethoxy)cyclohexyl]-6-(6-methoxypyridin-3-yl)-4-methylpyrido[2,3-d]pyrimidin-7(8H)-one (PF-04691502), duvelisib (ABBV-954), N2-[4-oxo-4-[4-(4-oxo-8-phenyl-4H-1-benzopyran-2-yl)morpholin-4-ium-4-ylmethoxy]butyryl]-L-arginyl-glycyl-L-aspartyl-L-serine acetate (SF-1126), pictilisib (GDC-0941), 2-methyl-1-[2-methyl-3-(trifluoromethyl)benzyl]-6-(morpholin-4-yl)-1H-benzimidazole-4-carboxylic acid (GSK2636771), idelalisib (GS-1101), umbralisib tosylate (TGR-1202), pictilisib (GDC-0941), copanlisib hydrochloride (BAY 84-1236), dactolisib (BEZ-235), 1-(4-[5-[5-amino-6-(5-tert-butyl-1,3,4-oxadiazol-2-yl)pyrazin-2-yl]-1-ethyl-1H-1,2,4-triazol-3-yl]piperidin-1-yl)-3-hydroxypropan-1-one (AZD-8835), 5-[6,6-dimethyl-4-(morpholin-4-yl)-8,9-dihydro-6H-[1,4]oxazino[4,3-e]purin-2-yl]pyrimidin-2-amine (GDC-0084) everolimus, rapamycin, perifosine, sirolimus, temsirolimus); a SOS1 inhibitor (e.g., BI-3406), a signal transduction pathway inhibitor (e.g., Ras-Raf-MEK-ERK pathway inhibitors, PI3K-Akt-mTOR-S6K pathway inhibitors (PI3K inhibitors)), a CTLA-4 inhibitor (e.g., ipilimumab, tremelimumab), an apoptosis pathway modulator (e.g., camptothecin), a cytotoxic chemotherapeutic agent (e.g., irinotecan), an anti-angiogenesis agent (e.g., paclitaxel, axitinib, bevacizumab, cabozantinib, everolimus, lenalidomide, lenvatinib mesylate, pazopanib, ramucirumab, regorafenib, sorafenib, sunitinib, thalidomide, vandetanib, ziv-aflibercept), a PD-1 and/or PD-L1 inhibitor (e.g., atezolizumab, avelumab, durvalumab, KN035, cosibelimab (CK-301), AUNP12, CA-170, BSM-986189, cemiplimab, dostarlimab, nivolumab, pembrolizumab (MK-3475), vopratelimab (JTX-4014), spartalizumab (PDR001), camrelizumab (SHR1210), sintilimab (IBI308), tislelizumab (BGB-A317), toripalimab (JS 001), INCMGA00012 (MGA012), AMP-224, AMP-514), an immune-check-point regulator (e.g., modulators of PD-1, PD-L1, B7H2, B7H4, CTLA-4, CD80, CD86, LAG-3, TIM-3, KIR, IDO, CD19, OX40, 4-1BB (CD137), CD27, CD70, CD40, GITR, CD28 and ICOS (CD278), CCL2, CCR2), a topoisomerase inhibitor (e.g., camptothecin derivatives such as topotecan and irinotecan, anthracyclines such as doxorubicin and daunorubicin, epipodophyllotoxins such as etoposide and teniposide, flavopiridol, ixabepilone, belomustine, lurtotecan), an ataxia telangiectasia and Rad3 related (ATR) kinase inhibitor (e.g., berzosertib (VX-970, M6620), VE-821, AZD6738, KU-60019, BAY-59-8862), a VEGF receptor tyrosine kinase inhibitor (e.g., tivozanib), a CDK4 and/or CDK6 inhibitor (e.g., ribociclib), a receptor tyrosine kinase RET (rearranged during transfection) inhibitor (e.g., selpercatinib), a KIT proto-oncogene receptor tyrosine kinase (KIT) inhibitor (e.g., ripretinib), a mTOR inhibitor (e.g., sirolimus, temsirolimus, ridaforolimus, AZD2014), a selective inhibitor of nuclear export (SINE) (e.g., selinexor), an ABL inhibitor (e.g., imatinib, nilotinib, dasatinib, busatinib, ponatinib), and an agent capable of interfering with an interaction between PD-1 and PD-L1 (e.g., any of the compounds disclosed in WO 2022/175955).

According to some of any of the embodiments described herein, the at least one therapeutically active agent is or comprises an agent that downregulates an activity of MEK and/or BRAF.

According to some of any of the embodiments described herein, the at least one agent that downregulates an activity of MEK and/or BRAF is selected from pimasertib, binimetinib, cobimetinib, refametinib, selumetinib, trametinib, mirdametinib (PD325901), PD318088.

PD334581, PD98059, PD184352 (CI-1040), AZD6244 (ARRY-142886), RDEA119, MEK162 (ARRY-438162), encorafenib (LGX818), dabrafenib, vemurafenib, sorafenib, GDC-0879, N-[3-(5-chloro-1H-pyrrolo[2,3-b]pyridin-3-ylcarbonyl)-2,4-difluorophenyl]propane-1-sulfonamide (PLX4720), (3R)—N-(3-[[5-(2-cyclopropylpyrimidin-5-yl)-1H-pyrrolo[2,3-b]pyridin-3-yl]carbonyl]-2,4-difluorophenyl)-3-fluoropyrrolidine-1-sulfonamide (PLX8394), and a structural analog thereof.

According to some of any of the embodiments described herein, the at least one therapeutically active agent comprises an immune checkpoint inhibitor.

According to some of any of the embodiments described herein, the at least one therapeutically active agent is or comprises an agent that interferes with an activity or expression of PD1 and/or PDL1, and/or an agent that interferes with an interaction between PD1 and PDL1.

According to some of any of the embodiments described herein, the at least one therapeutically active agent comprises a PARP inhibitor.

According to some of any of the embodiments described herein, the at least one therapeutically active agent comprises a topoisomerase 1 inhibitor.

According to some of any of the embodiments described herein, the composition comprises at least two of the therapeutically active agents.

According to some of any of the embodiments described herein, the at least two therapeutically active agents act in synergy in treating the medical condition.

According to some of any of the embodiments described herein, in at least a portion of the particles, each particle comprises the at least two therapeutically active agents.

According to some of any of the embodiments described herein, in at least a portion of the particles each particle comprises a first therapeutically active agent and in at least another portion of the particles each particle comprises a second therapeutically active agent, and wherein the first and second therapeutically active agents act in synergy.

According to some of any of the embodiments described herein, in at least a portion of the particles, each particle comprises at least one agent that downregulates an activity of MEK and at least one agent that downregulates an activity of BRAF.

According to some of any of the embodiments described herein, one portion of the particles comprises an agent that downregulates an activity of MEK as the therapeutically active agent and another portion of the particles comprises an agent that downregulates an activity of BRAF as the therapeutically active agent.

According to some of any of the embodiments described herein, the agent that downregulates an activity of MEK and the agent that downregulates an activity of BRAF act in synergy.

According to some of any of the embodiments described herein, the agent that downregulates the activity of MEK is trametinib.

According to some of any of the embodiments described herein, the agent that downregulates the activity of BRAF is dabrafenib.

According to some of any of the embodiments described herein, at least one of the agent that downregulates an activity of MEK and the agent that downregulates an activity of BRAF is used in a sub-therapeutically effective amount.

According to some of any of the embodiments described herein, in at least a portion of the particles, each particle comprises the at least one PARP inhibitor and at least one agent that that interferes with an activity or expression of PD1 and/or PDL1, and/or an agent that interferes with an interaction between PD1 and PDL1.

According to some of any of the embodiments described herein, one portion of the particles comprises the at least one PARP inhibitor as the therapeutically active agent and another portion of the particles comprises at least one agent that that interferes with an activity or expression of PD1 and/or PDL1, and/or an agent that interferes with an interaction between PD1 and PDL1 as the therapeutically active agent.

According to some of any of the embodiments described herein, the PARP inhibitor and the at least one agent that that interferes with an activity or expression of PD1 and/or PDL1, and/or an agent that interferes with an interaction between PD1 and PDL1 act in synergy.

According to some of any of the embodiments described herein, in at least a portion of the particles, each particle comprises the at least one PARP inhibitor and at least one topoisomerase inhibitor.

According to some of any of the embodiments described herein, one portion of the particles comprises the at least one PARP inhibitor as the therapeutically active agent and another portion of the particles comprises at least one topoisomerase inhibitor as the therapeutically active agent.

According to some of any of the embodiments described herein, the PARP inhibitor and the topoisomerase inhibitor act in synergy.

According to some of any of the embodiments described herein, the composition is a pharmaceutical composition that further comprises a pharmaceutical acceptable carrier.

According to an aspect of some embodiments of the present invention there is provided a composition as described herein in any of the respective embodiments and any combination thereof, for use in treating the medical condition in a subject in need thereof, as described herein in any of the respective embodiments and any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawings will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

### In the Drawings:

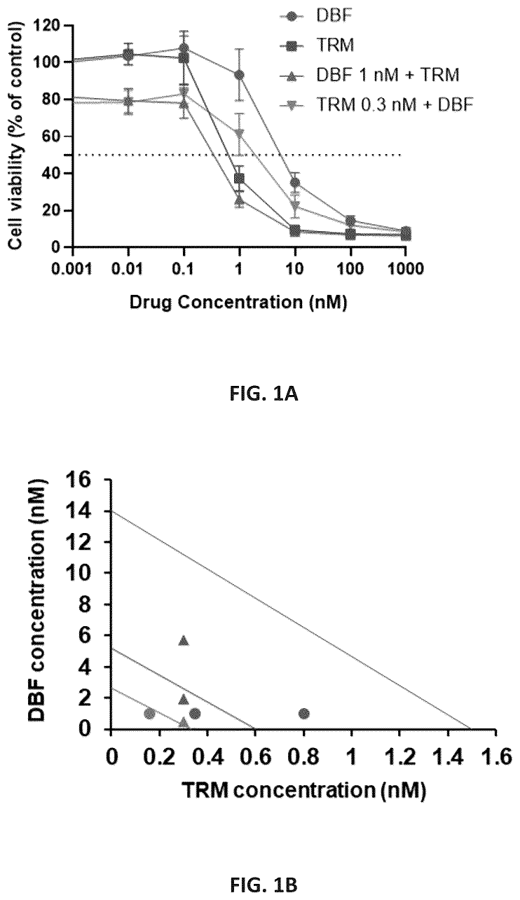

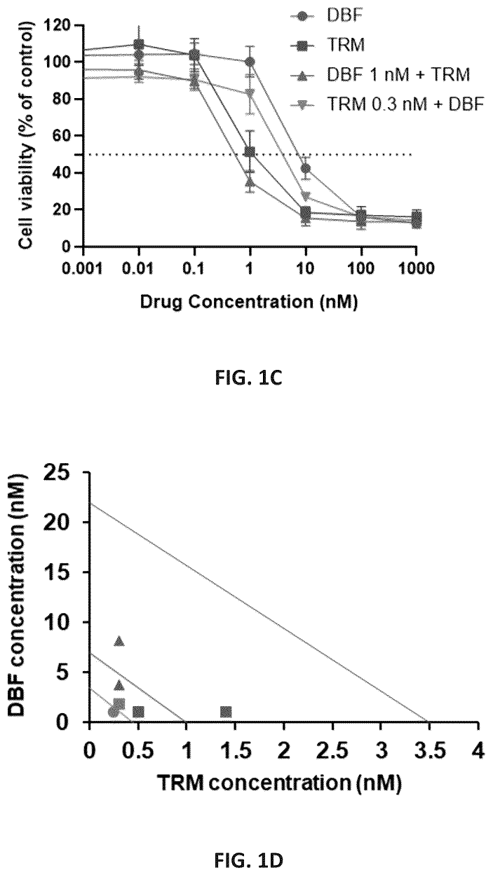

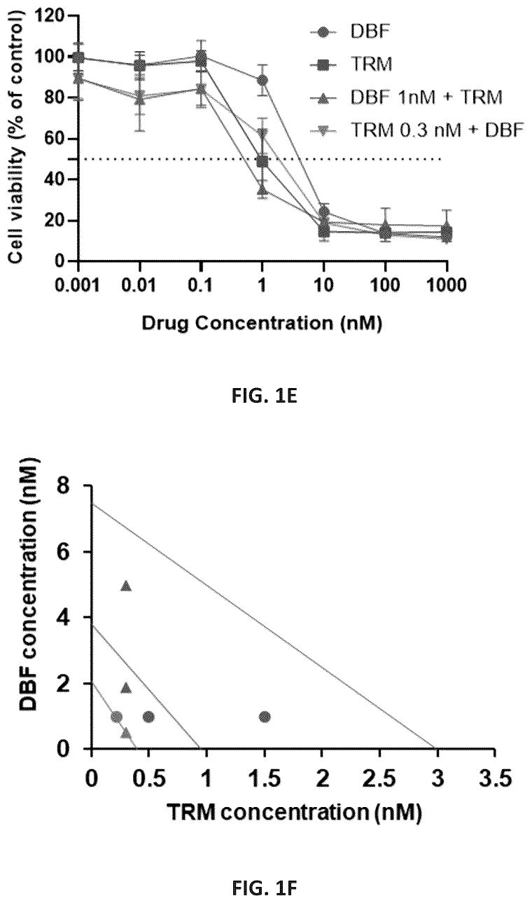

FIGS. 1A-F are comparative plots showing inhibition of cell proliferation (FIGS. 1A, 1C, and 1E) and respective isobolograms of DBF and TRM drug combination (FIGS. 1B, 1D, and 1F) on different cell lines: murine D4M.3A (FIGS. 1A-B), human 131/4-5B1 (FIGS. 1C-D), and human A375 (FIGS. 1E-F) cell lines. Data represent mean±SD of triplicate wells and the graphs summarize three independent experiments.

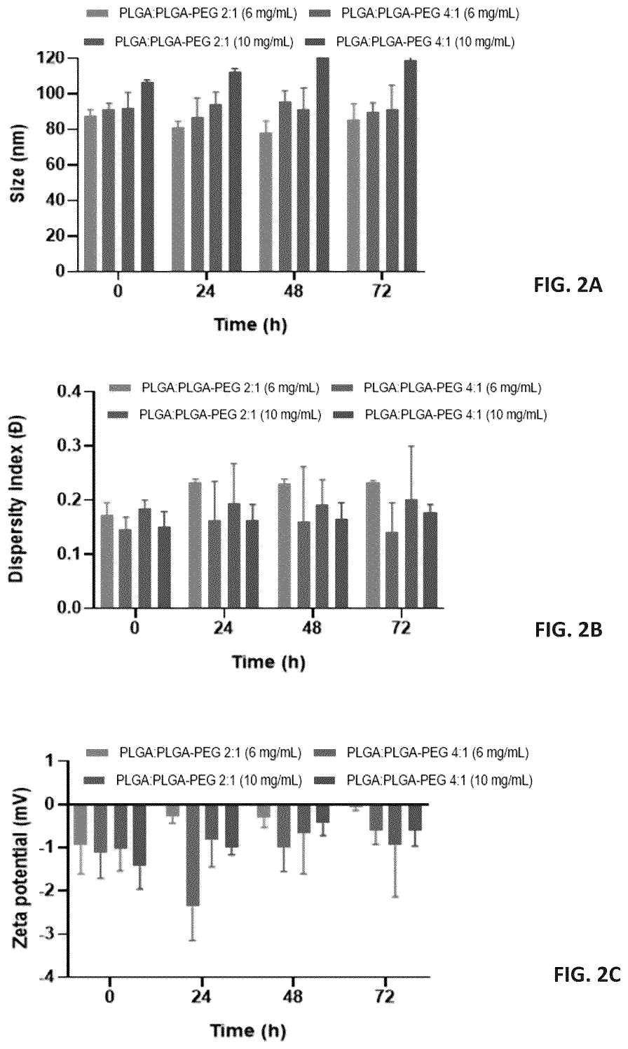

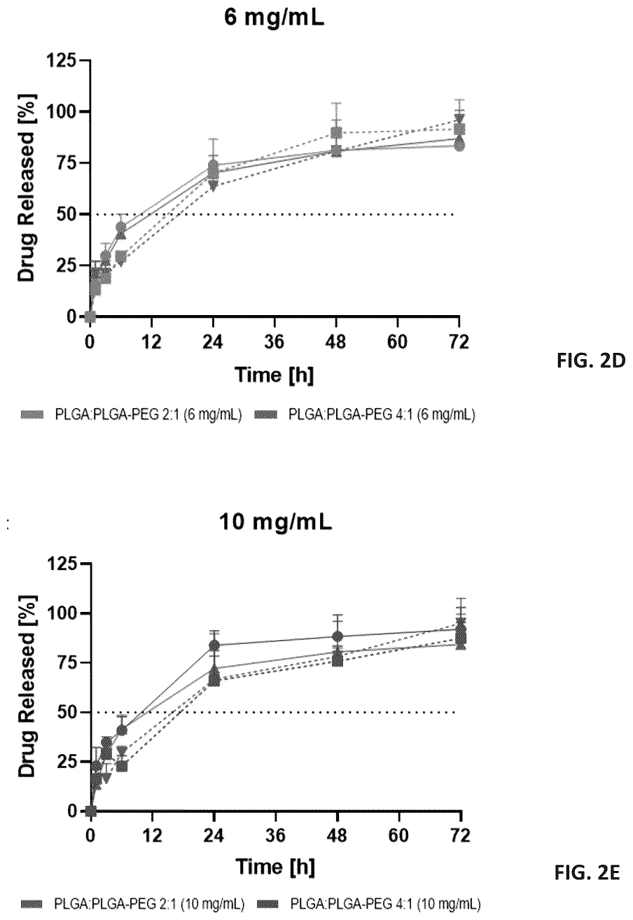

FIGS. 2A-C are bar graphs showing stability data of nanoparticles comprising a ratio of 2:1 (green and red graphs) or 4:1 (purple and blue graphs) PLGA: PLGA-PEG at 6 mg/mL (green and purple graphs) or 10 mg/mL (red and blue graphs) polymer concentration, as measured by DLS: hydrodynamic size (FIG. 2A), dispersity index (FIG. 2B), and zeta potential (FIG. 2C). The NPs were suspended in PBS and gently stirred for 72 hours at 37° C.

FIGS. 2D and 2E are comparative plots presenting release profiles of DBF (solid line) and TRM (dashed line), at low (6 mg/mL; FIG. 2D) and high (10 mg/mL; FIG. 2E) concentrations of PLGA in the tested nanoparticles from FIGS. 2A-C. The NPs were aliquoted into 200 μL dialysis tubes and incubated in 1 Liter PBS at 37° C. At predetermined time points, 200 μL of targeted NPs were collected, lyophilized, and analyzed for drug content. The data represent the average of three independent repeats, and it signifies mean±SD.

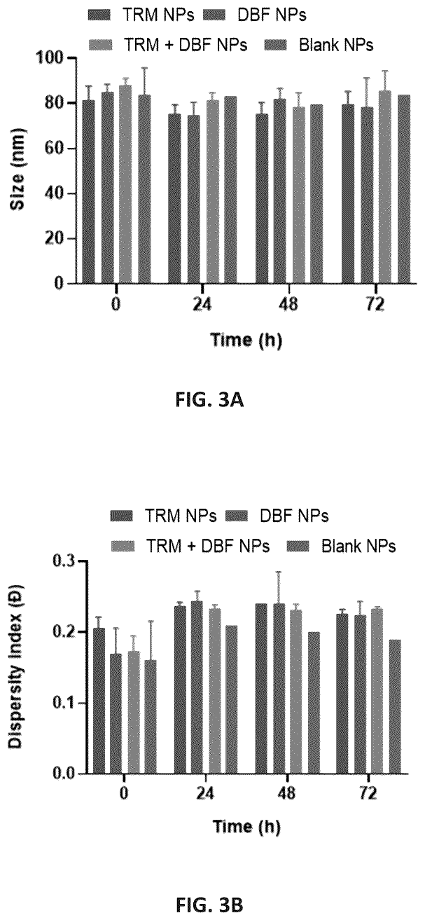

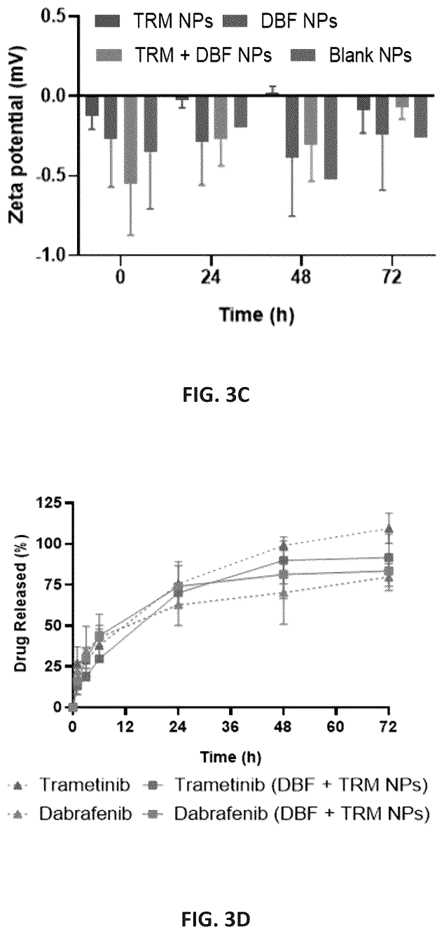

FIGS. 3A-C are bar graphs showing stability of nanoparticles formulation comprising 6 mg of polymers at a 2:1 PLGA: PLGA-PEG ratio (6 mg/mL polymer concentration) formulated with 0.1 mg TRM (blue), 0.9 mg DBF (red), 0.1 mg TRM and 0.9 mg DBF (green), or in the absence of TRM and/or DBF (blank; purple), as measured by DLS: NPs size (FIG. 3A), dispersity index (FIG. 3B), and zeta potential (FIG. 3C).

FIG. 3D are comparative plots presenting release profiles of DBF and TRM single-loaded and dual-loaded DBF+TRM NPs, as described in FIGS. 3A-C.

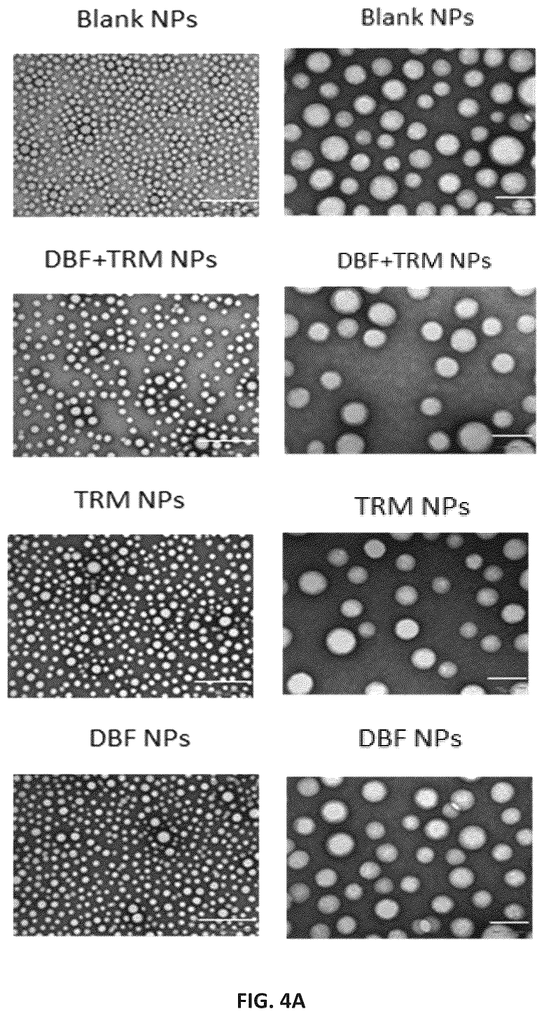

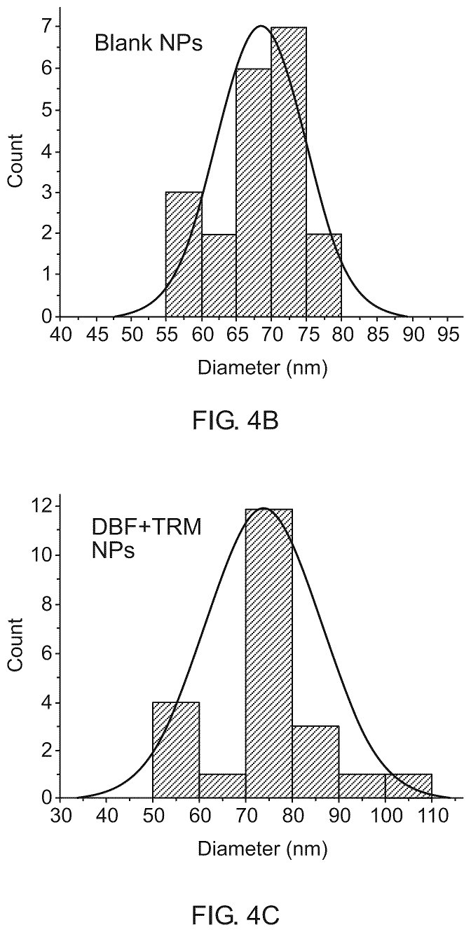

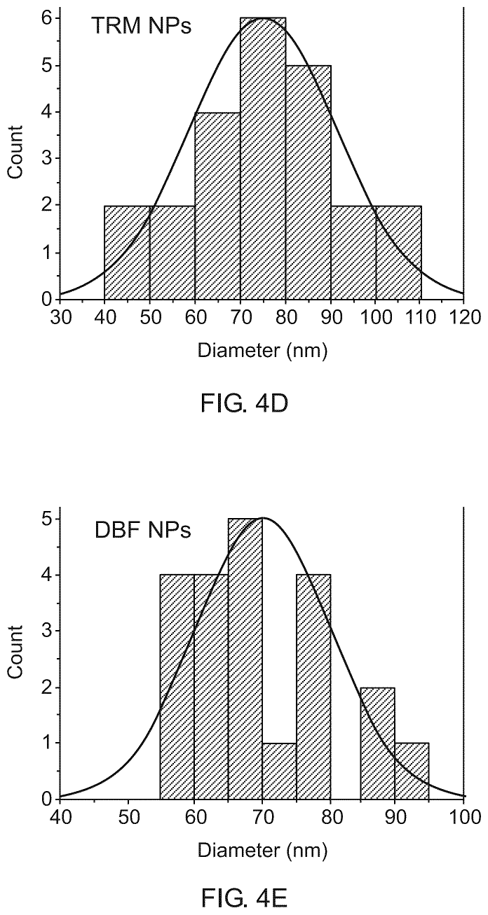

FIG. 4A are representative TEM images and size distribution of blank (empty) NPs, dual-loaded DBF and TRM NPs, single-loaded TRM NPs, and single-loaded DBF NPs, as indicated, as described in FIGS. 3A-C. Scale bars are 500 nm (left-side insets) and 100 nm (right-side insets).

FIGS. 4B-E are diameter distribution images of NPs formulated as described in FIGS. 3A-C, as produced by ImageJ software using the corresponding TEM images in FIG. 4A. The Data represent mean±SD.

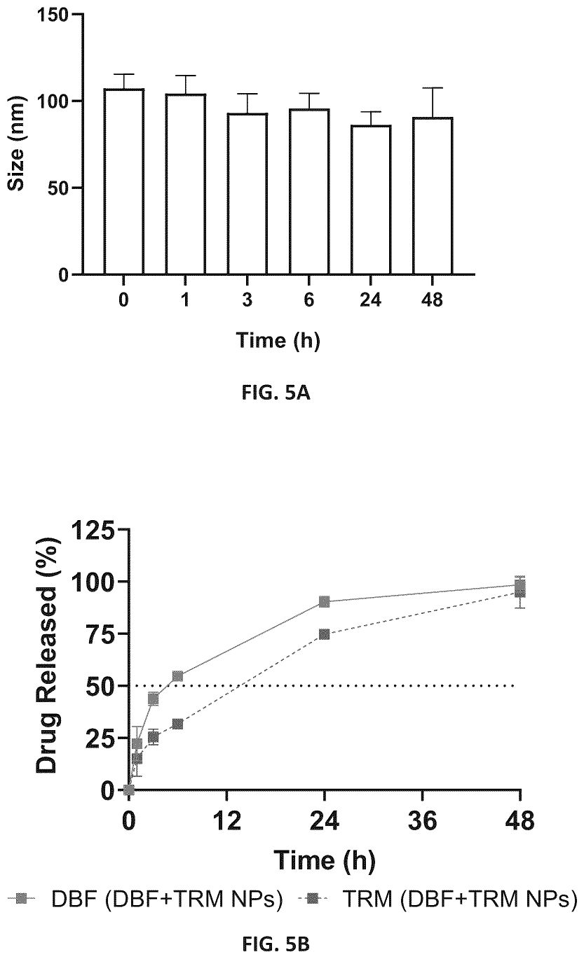

FIG. 5A is a bar graph presenting the time-dependent size of nanoparticles formulation comprising 6 mg of polymers at a 2:1 PLGA: PLGA-PEG ratio (6 mg/mL polymer concentration) formulated with 0.1 mg TRM and 0.9 mg DBF, as measured by DLS, indicating the stability of the NPs. The NPs were suspended in DMEM medium containing 10% FBS and were gently stirred for 48 hours at 37° C.

FIG. 5B is a comparative plot presenting release profiles of DBF and TRM from dual-loaded DBF+TRM NPs, as described in FIG. 5A. The NPs were suspended in DMEM medium containing 10% FBS and gently stirred for 48 hours at 37° C.

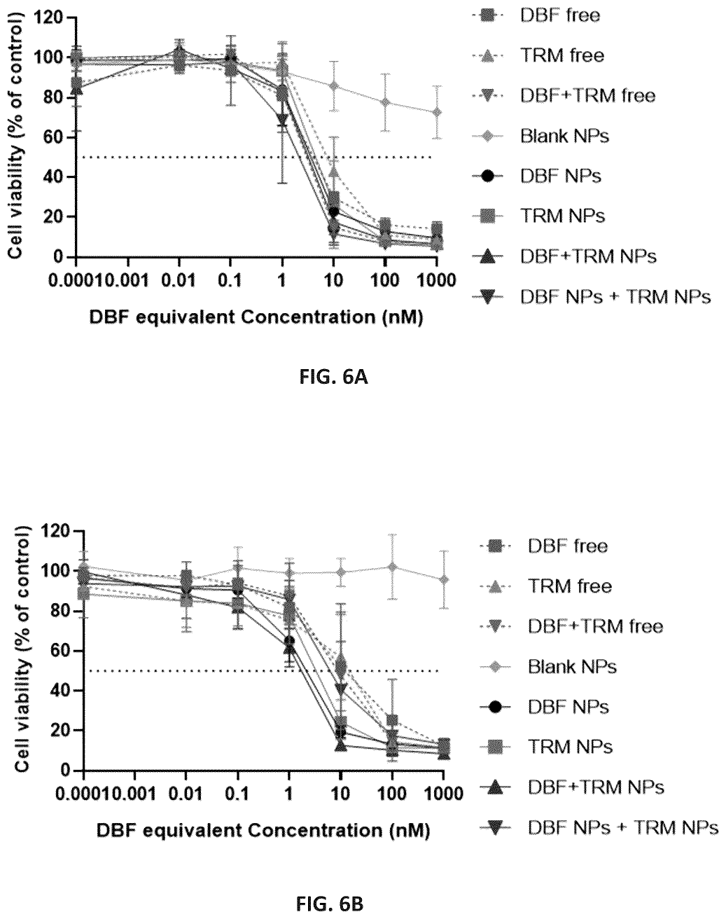

FIGS. 6A and 6B are comparative plots showing the dose-dependent murine D4M.3A (FIG. 6A) and human A375 (FIG. 6B) melanoma cell viability following treatment with DBF- and/or TRM-drug-loaded NPs or free drugs at serially diluted concentrations for 72 hours and growth inhibition was evaluated. The data represent mean±SD, N=3.

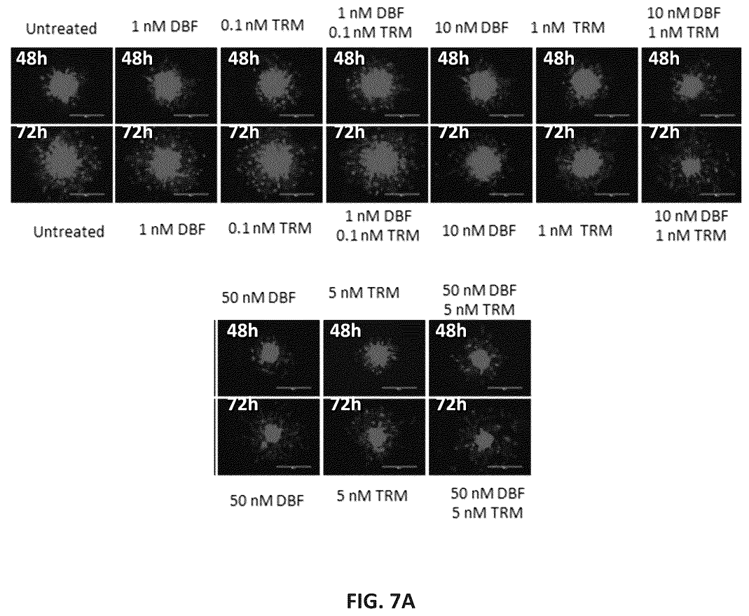

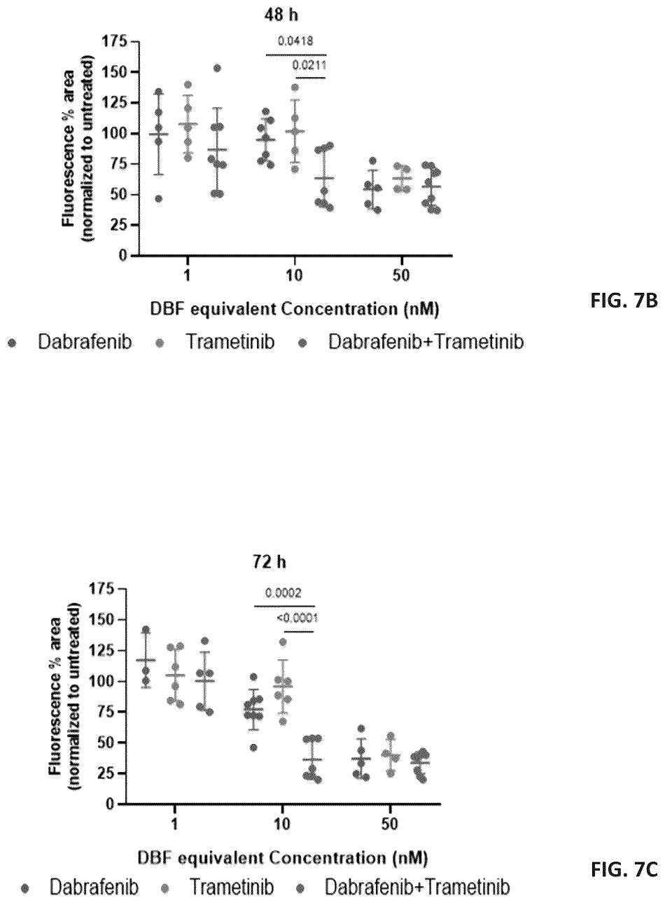

FIG. 7A are representative images of dose-dependent effect of free drugs combination on the sprouting of mCherry-labeled human WM115 melanoma 3D tumor spheroids, 48 hours and 72 hours post-treatment. The spheroids were imaged 48-72 hours post-treatment with free drugs. The cells were incubated in “hanging drop” for 72 hours, then the spheroids were transferred to Matrigel® and treated with free DBF, TRM, and their combination at a 10:1 ratio, respectively. At least 3 spheroids were imaged for each treatment. Data are shown as mean±SD. Scale bar is 400 μm. Statistical analysis was performed using two-sided repeated-measures ANOVA (p-value <0.05).

FIGS. 7B and 7C are quantification graph of mCherry % fluorescent area of WM115 melanoma 3D tumor spheroids, at 48 hours (FIG. 7B) and 72 hours (FIG. 7C) post-treatment, as described in FIG. 7A.

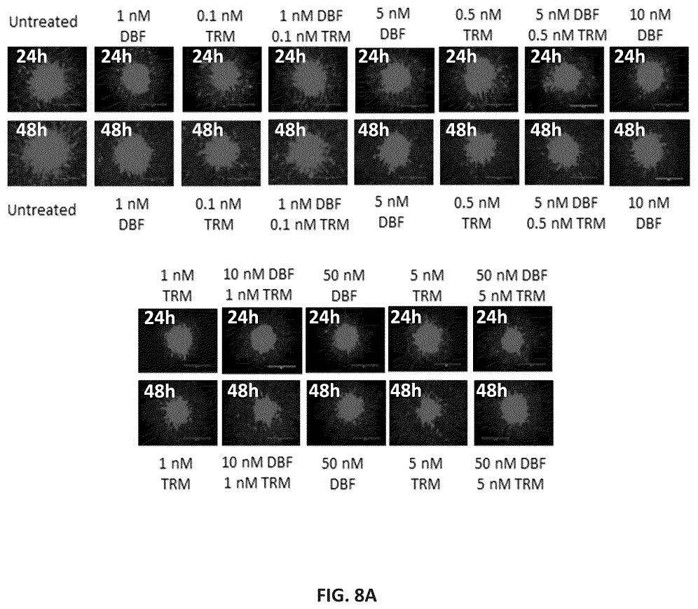

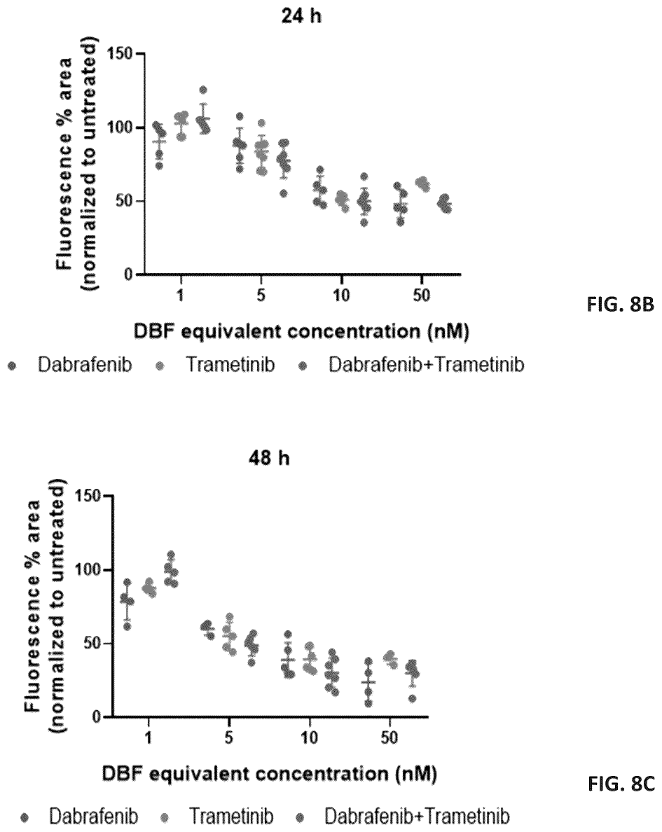

FIG. 8A are representative images of dose-dependent effect of free drugs combination on the sprouting of mCherry-labeled murine D4M.3A melanoma 3D tumor spheroids, 24 hours and 48 hours post-treatment. The spheroids were imaged 24-48 hours post-treatment with free drugs. The cells were incubated in “hanging drop” for 72 hours, then the spheroids were transferred to Matrigel® and treated with free DBF, TRM, and their combination at a 10:1 ratio, respectively. At least 3 spheroids were imaged for each treatment. Data are shown as mean±SD. Scale bar is 400 μm. Statistical analysis was performed using two-sided repeated-measures ANOVA (p-value <0.05).

FIGS. 8B and 8C are quantification graph of mCherry % fluorescent area of D4M.3A melanoma 3D tumor spheroids, as described in FIG. 8A.

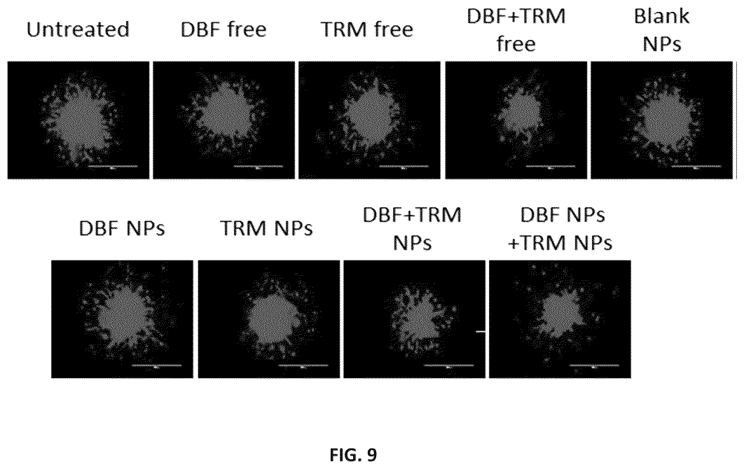

FIG. 9 are representative images of mCherry-labeled WM115 spheroids following 48 hours incubation with drug-loaded NPs formulated using a ratio of 2:1 PLGA: PLGA-PEG at 6 mg/mL polymer concentration in the presence of 10 nM DBF and 1 nM TRM or free drugs at 10 nM DBF and 1 nM TRM, separately or combined. At least 3 spheroids were imaged for each treatment, the assay was repeated thrice. Scale bar is 400 μm.

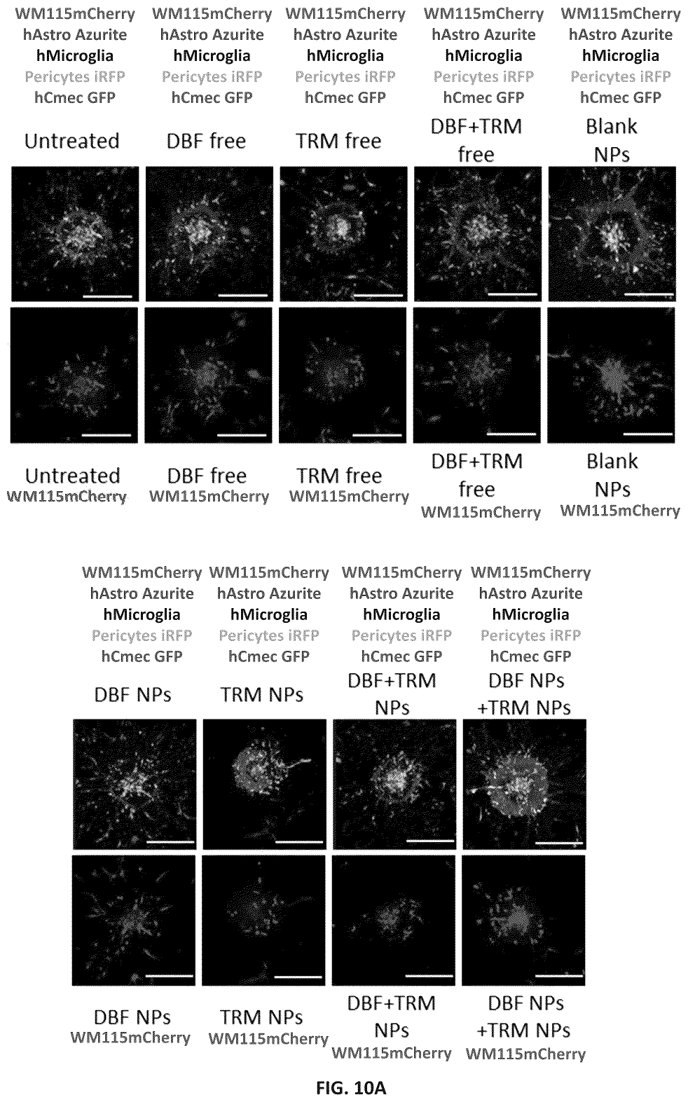

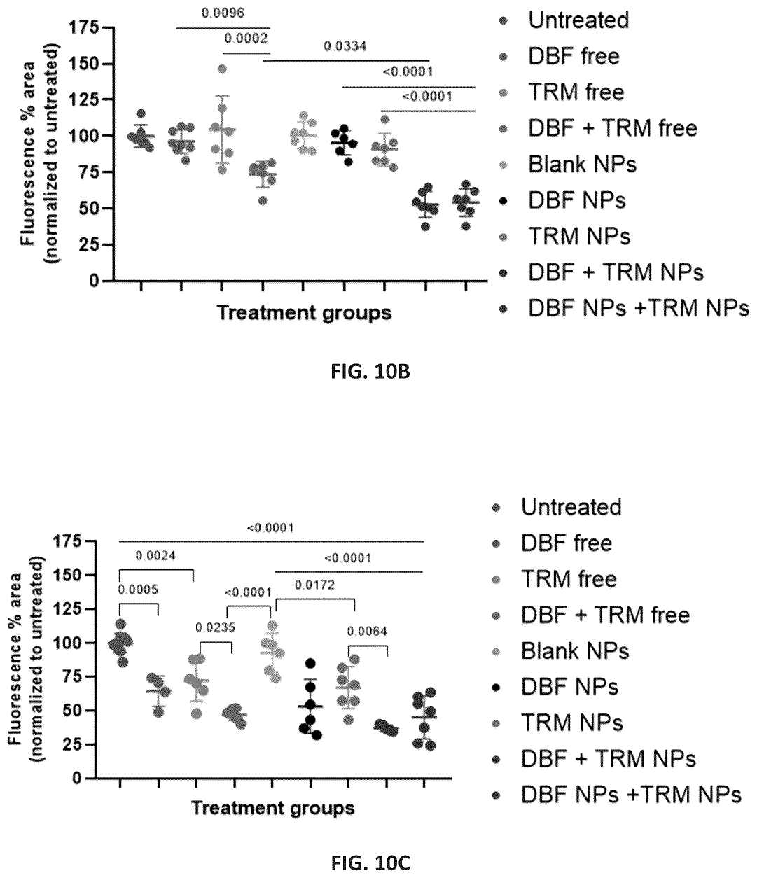

FIG. 10A are confocal images of multicellular spheroids. The spheroids contained tumor cells (WM115 mCherry), astrocytes (hAstro azurite), microglia (hMicroglia), pericytes (hPericytes iRFP) and endothelial cells (hCMEC GFP) at 1:1:0.5:0.5:2 ratio, respectively, and were treated with 10 nM DBF and 1 nM TRM for 72 hours. At least 3 spheroids were imaged for each treatment, the assay was repeated twice. Scale bar is 400 μm.

FIGS. 10B and 10C are quantification graphs of mCherry % fluorescent area of WM115 spheroids (FIG. 9) and melanoma brain metastasis (MBM) multicellular tumor spheroids (MCTS) (FIG. 10A) untreated or treated with free drugs (10 nM DBF, 1 nM TRM, each alone and combined) or nanoparticles (formulated using a ratio of 2:1 PLGA: PLGA-PEG at 6 mg/mL polymer concentration; blank or loaded with DBF and/or TRM, at 10 nM DBF and/or 1 nM TRM, or combined DBF nanoparticles and TRM nanoparticles).

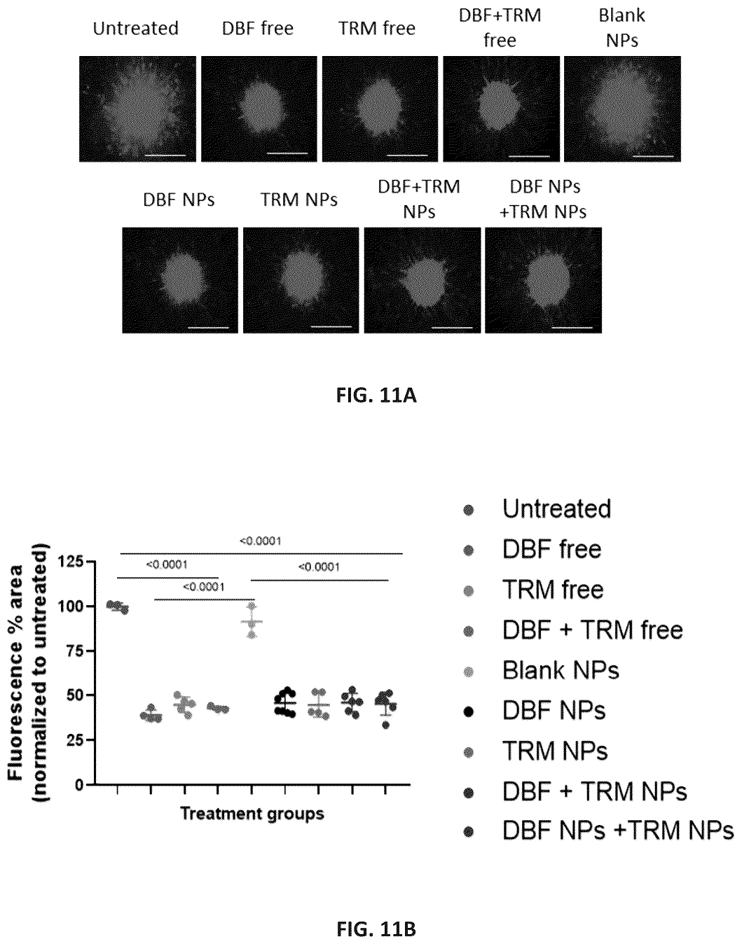

FIG. 11A are representative images of D4M.3A mCherry-labeled spheroids, following 48 hours incubation with drug-loaded NPs formulated as described in FIG. 9 or free drugs.

FIG. 11B is quantification graph of mCherry % fluorescent area of D4M.3A spheroids untreated or treated with free drugs (10 nM DBF, 1 nM TRM, each alone and combined) or nanoparticles (blank, NPs loaded with DBF and/or TRM, at 10 nM DBF and/or 1 nM TRM, or combined DBF nanoparticles and TRM nanoparticles). Data are shown as mean±SD. Scale bar is 400 μm. All Statistical analysis was performed using two-sided repeated-measures ANOVA (p-value <0.05) and the quantification was made for the mCherry-labeled tumor cells using ImageJ.

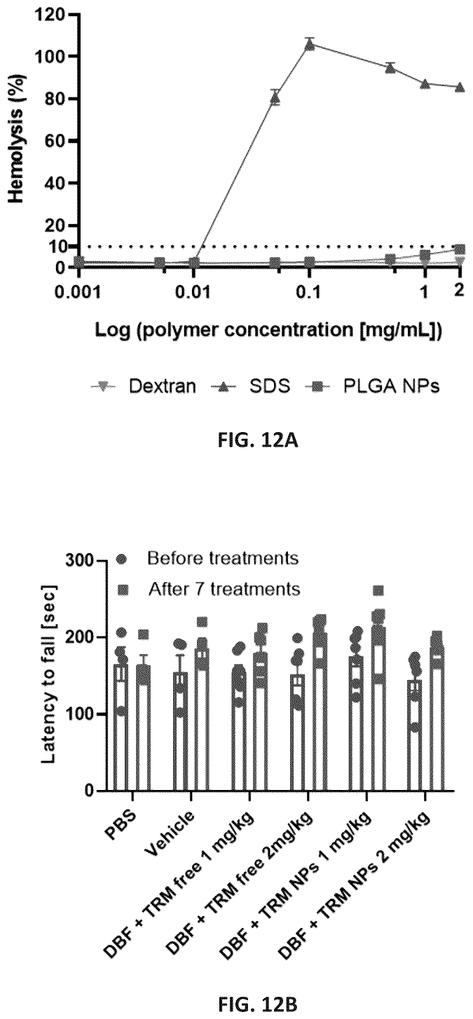

FIG. 12A is a comparative plot, showing dose-dependent hemolysis % in the presence of various polymers. Red blood cells (RBC) were treated with increasing concentrations of PLGA NPs, dextran (negative control), or sodium dodecyl sulfate (SDS, positive control) for 1 hour, and the percentage of hemoglobin released was measured using a spectrophotometer.

FIG. 12B are bar charts, showing the effect of systemic administration of free drugs and drug-loaded PLGA NPs on the motor performance of the treated mice. Data are presented as mean±SEM. Individual measurements represent the average of the 3 longest performances out of 5 trials. N=4 mice for PBS and vehicle groups and N=6-7 mice for the rest of the groups.

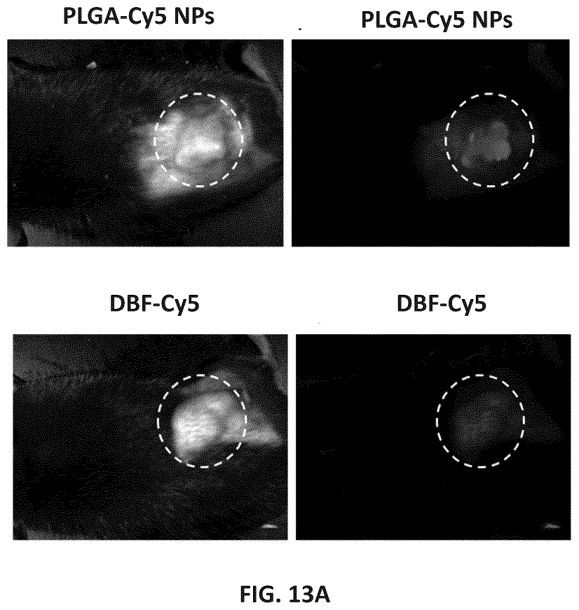

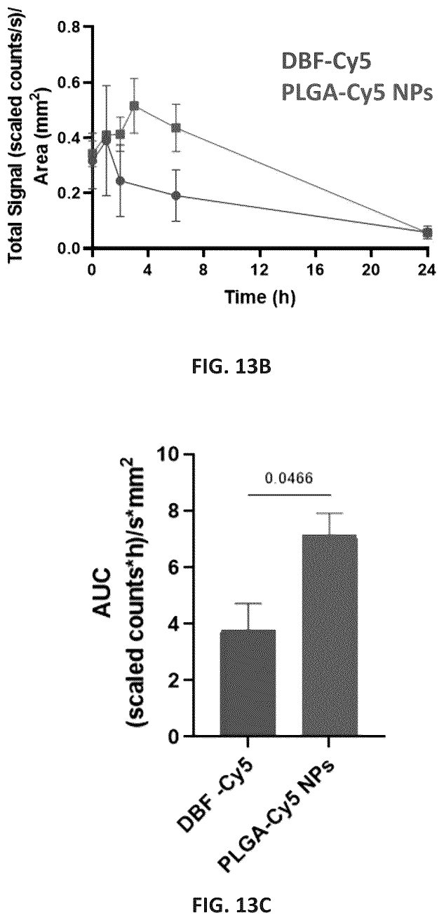

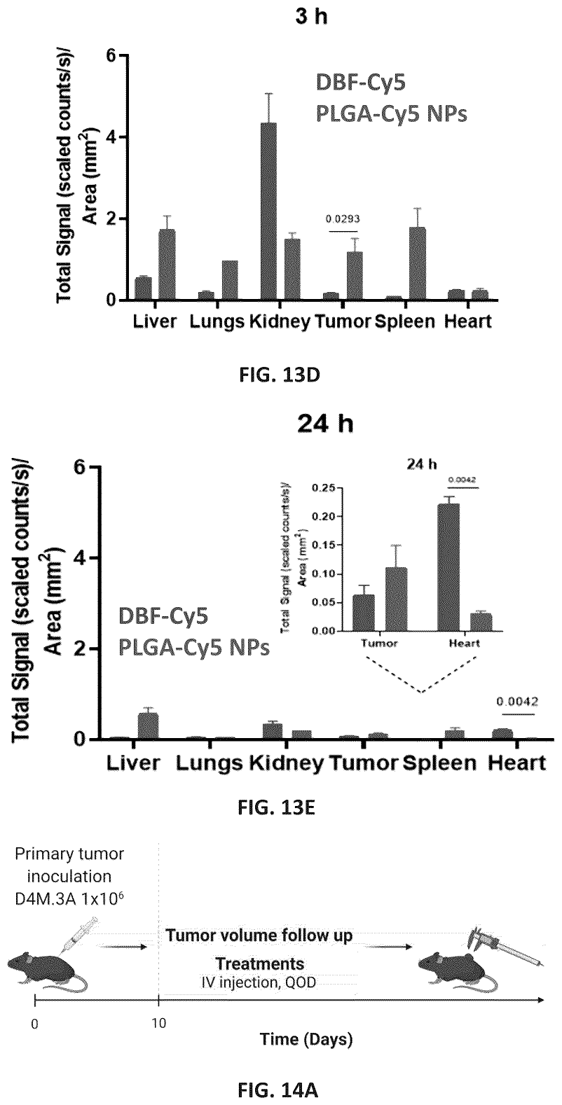

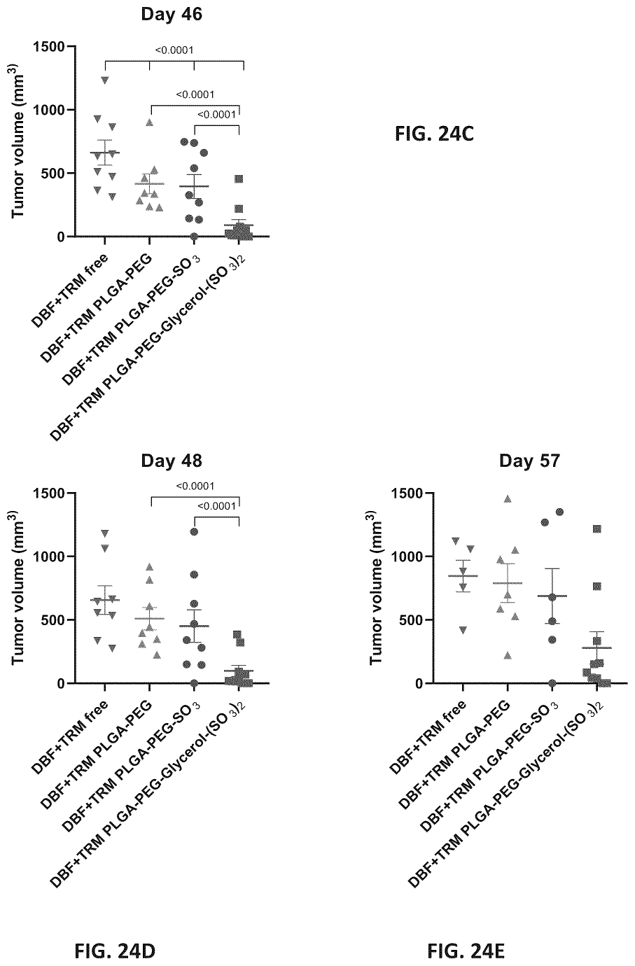

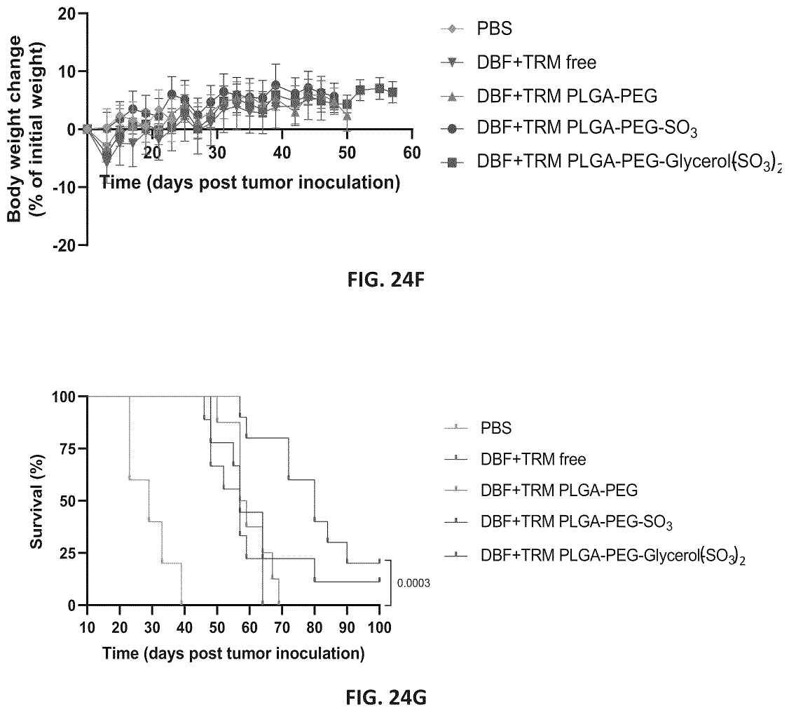

FIGS. 13A-E present comparative data of total fluorescent signal following IV administration of Cy5-labeled PLGA-NPs (PLGA-Cy5 NPs; red) and DBF (DBF-Cy5; blue) to examine the tumor accumulation of PLGA nanoparticles in comparison with the tumor accumulation of a free drug. FIG. 13A are representative images of Cy5 signal from D4M.3A tumors 6 hours after IV administration of PLGA-Cy5 NPs or DBF-Cy5, as indicated. FIG. 13B presents comparative plots showing tumor accumulation, following treatment of D4M.3A tumor-bearing mice with PLGA-Cy5 NPs or DBF-Cy5 at 20 μM Cy5-eq. concentration and monitored for 0, 1, 2, 3, 6, and 24 hours following the IV injection; FIG. 13C is a bar graph showing the total tumor accumulation. The area under the curve (AUC) was calculated for each treatment, 24 hours post the injection. N=3 mice per group and one-way ANOVA was used for statistical analysis. FIGS. 13D-E are bar graphs showing bio-distribution following 3 hours (FIG. 13D) and 24 hours (FIG. 13E) after the IV administration. The organ's fluorescent signal was normalized to its surface area (mm²). N=3 mice per group, two-way ANOVA was used for statistical analysis.

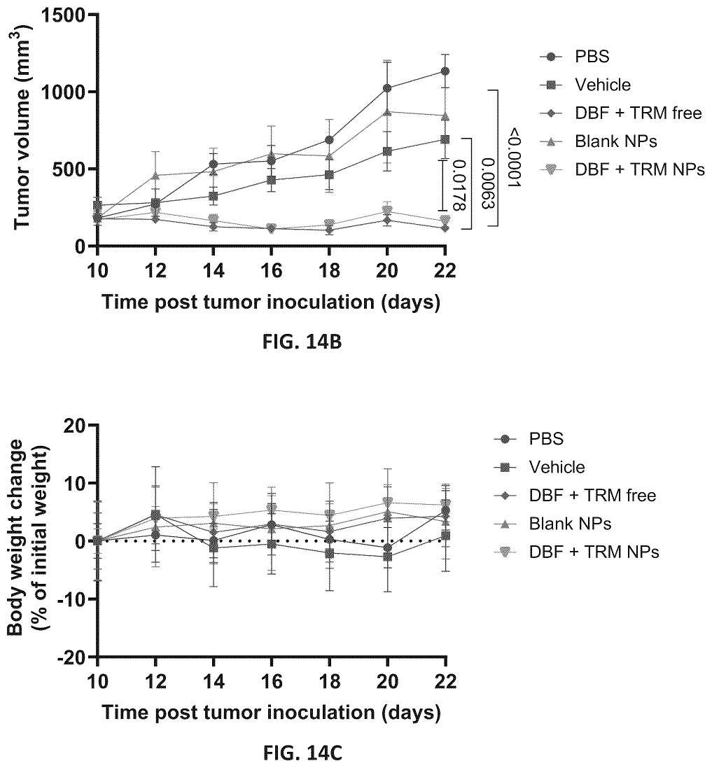

FIG. 14A is a schematic presentation of an exemplary study protocol, including timeline (days) of tumor inoculation, treatments, and follow-up, performed in order to evaluate the anti-tumor activity of drug-loaded PLGA nanoparticles in vivo. C57BL/6 Mice were inoculated with D4M.3A cells, were allowed to establish tumors for 10 days, following by treatment with free DBF and TRM or DBF- and TRM-loaded nanoparticles. The mice were treated until the control group (PBS) reached the endpoint of tumor size above 1000 mm³.

FIG. 14B are comparative plots presenting tumor volume growth curve of the different treatment groups, tested in the study described in FIG. 14A. Mice were treated QOD with IV injections of free drugs or drug-loaded NPs at 3 mg/kg DBF and 0.3 mg/kg TRM for 12 days. On day 22, the PBS group reached the endpoint of tumor size above 1000 mm³ and the mice were euthanized to collect the tumors for immunohistochemical staining. The data indicate that combined therapies reduce tumor size compared to the controls.

FIG. 14C are comparative plots presenting bodyweight change curve following different treatment courses, tested in the study described in FIGS. 14A-B. The body weight was monitored throughout the treatments and was normalized to the mice's weight before treatments initiation as a safety evaluation. No substantial change in bodyweight was observed. Data represent mean t SEM. Statistical significance was determined using two-sided repeated-measures ANOVA (p-value <0.05). N=5 mice per group, N=3 mice per vehicle group.

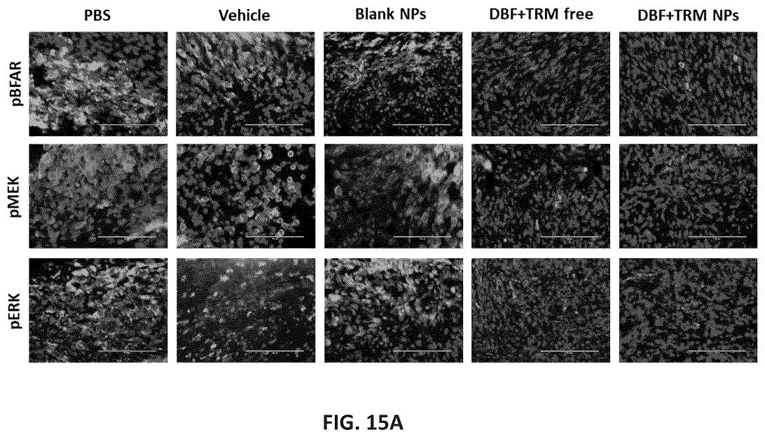

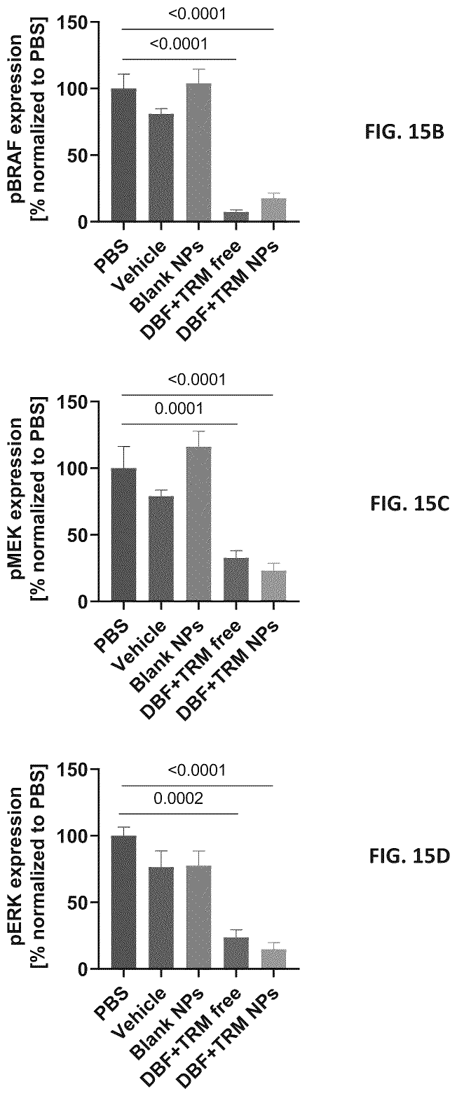

FIG. 15A are representative images of D4M.3A tumor tissues after the 7^{th} treatment described in FIGS. 14A-B, stained for phospho-BRAF, phospho-MEK1/2, phospho-ERK1/2 (FITC-green), and nucleus (Hoechst-blue), to assess the expression of phosphorylated kinases. Scale bar is 100 μm.

FIGS. 15B-D are quantification of pBRAF (FIG. 15B), pMEK1/2 (FIG. 15C), and pERK1/2 (FIG. 15D) in D4M.3A tumor tissues, as described in FIG. 15A. FITC % fluorescent covered area is represented as a relative change from the control (PBS) group. Statistical significance was determined using one-sided repeated-measures ANOVA (p-value <0.05).

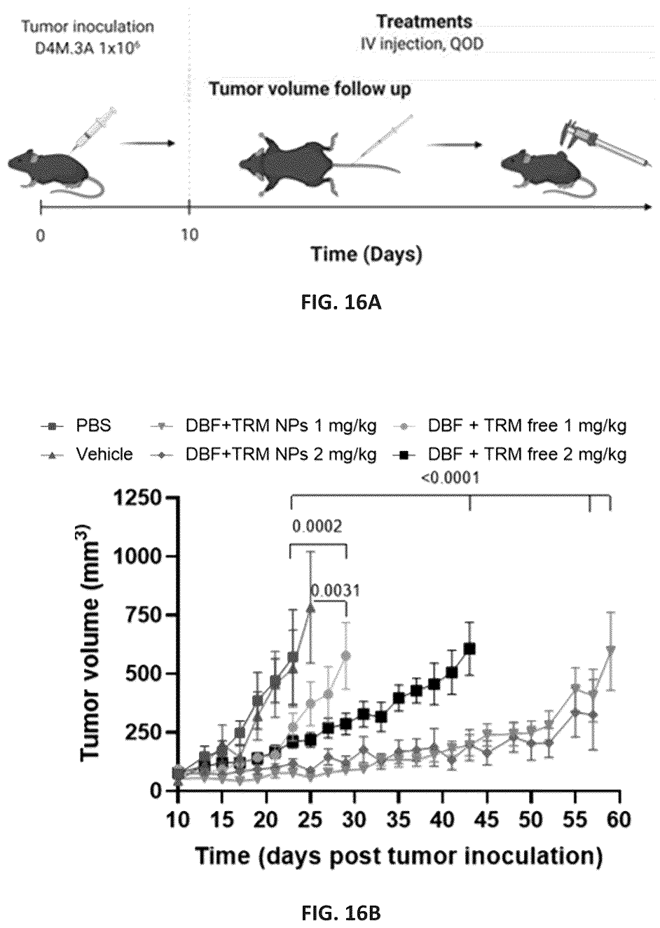

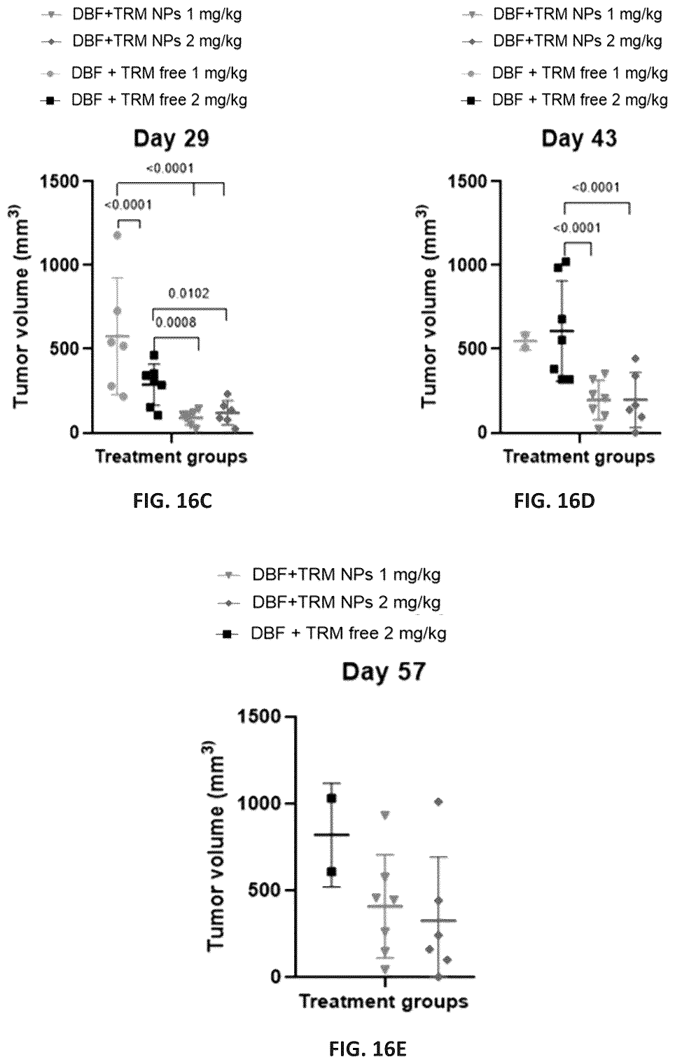

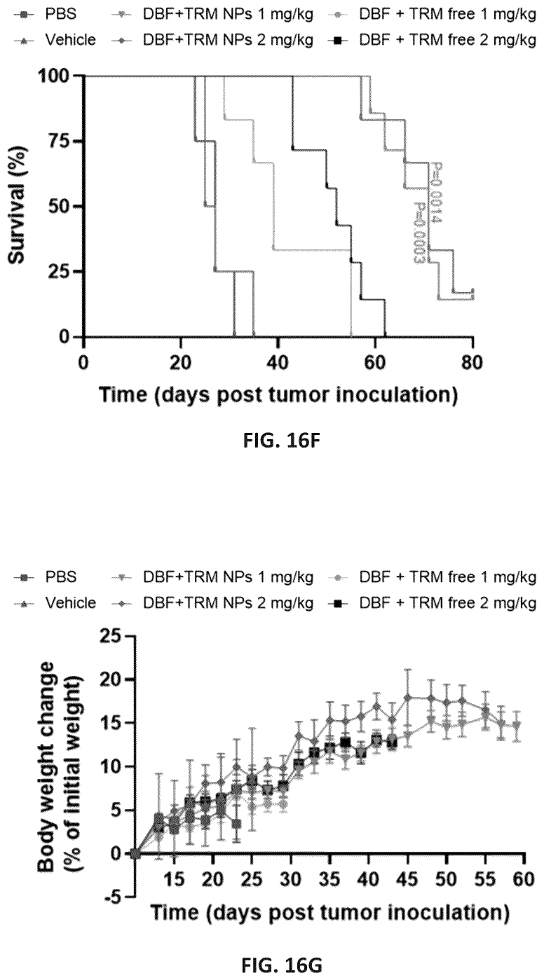

FIG. 16A is a schematic presentation of an exemplary study protocol, including timeline (days) of tumor inoculation, treatments, and follow-up, performed as a dose-reduction experiment in order to exploit the synergistic anti-tumor effect of DBF and TRM. C57BL/6 mice were inoculated with D4M.3A cells and were allowed to establish tumors until the average tumor volume reached 70 mm³ (day 10). The mice were then randomized into groups and treated systemically with the combination of DBF (2 or 1 mg/kg, QOD) and TRM (0.2 or 0.1 mg/kg, QOD) as free drugs or as drug-loaded PLGA nanoparticles.

FIG. 16B are comparative plots presenting tumor volume growth curve in different treatment groups, tested in the study described in FIG. 16A. P values correspond to tumor volume of the treatment groups compared to PBS at day 23 and to vehicle at day 25. The data show that the drug-loaded PLGA nanoparticles prolong survival of the mice compared to the controls and free drugs.

FIGS. 16C-E are quantifications of individual tumor volumes of the treatment groups, tested in the study described in FIG. 16A, at particular time points: at day 29 (FIG. 16C), day 43 (FIG. 16D), and day 57 (FIG. 16E). Statistical significance was calculated only for groups that retained the initial number of mice at the given time point. The data show that the drug-loaded PLGA nanoparticles reduce tumor size compared to the controls and free drugs. Data are presented as mean±SEM. Statistical significance was determined using two-sided repeated-measures ANOVA (p-value <0.05). N=4 mice for PBS and Vehicle groups, N=6-7 mice for the remaining groups.

FIG. 16F presents a Kaplan-Meier survival curve for mice inoculated with 1×10⁶ D4M.3A cells, as described in FIG. 16A. P values refer to NPs compared to free drugs at the same concentrations. In addition, P=0.0008 for DBF+TRM NPs 1 mg/kg compared with free DBF+TRM 2 mg/kg and for DBF+TRM NPs 2 mg/kg compared with free DBF+TRM 1 mg/kg. For PBS: P=0.0006 compared with DBF+TRM NPs 1 mg/kg and free DBF+TRM 2 mg/kg. P=0.0013 compared with DBF+TRM NPs 2 mg/kg. P=0.0054 compared with free DBF+TRM 1 mg/kg. For Vehicle: P=0.0005 compared with DBF+TRM NPs 1 mg/kg and free DBF+TRM 2 mg/kg. P=0.0011 compared with DBF+TRM NPs 2 mg/kg. P=0.0115 compared with free DBF+TRM 1 mg/kg. P values were determined using a log-rank test. The data show that both the drug-loaded PLGA nanoparticles prolong survival of the mice compared to the controls and free drugs.

FIG. 16G are comparative plots presenting bodyweight change curve following different treatment courses, tested in the study described in FIG. 16A. The mice's body weight was expressed as a percent change from the day of treatment initiation, as a safety evaluation. Up to 20% change in bodyweight was observed. Data represent mean±SEM.

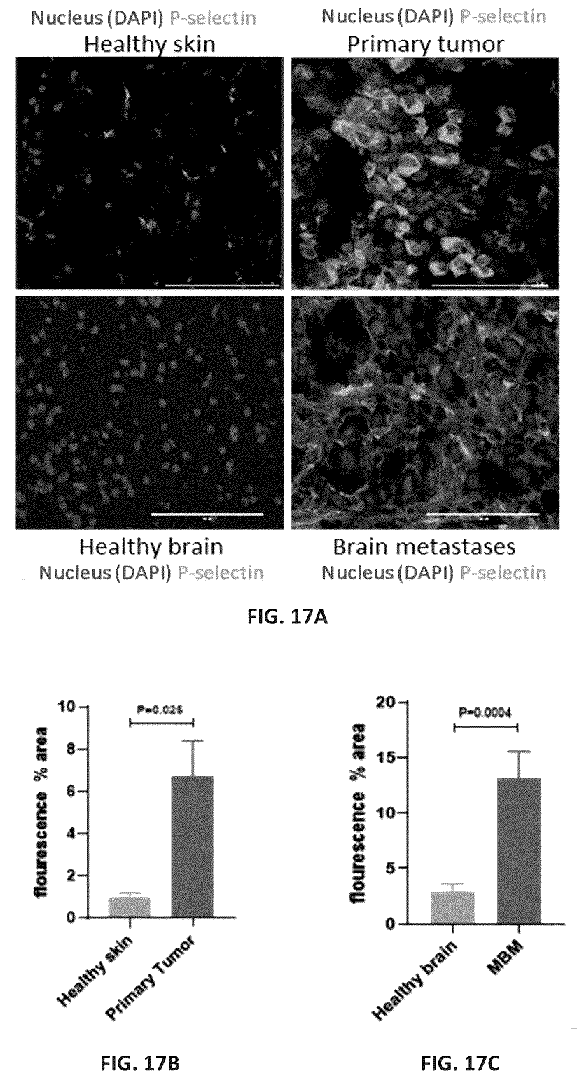

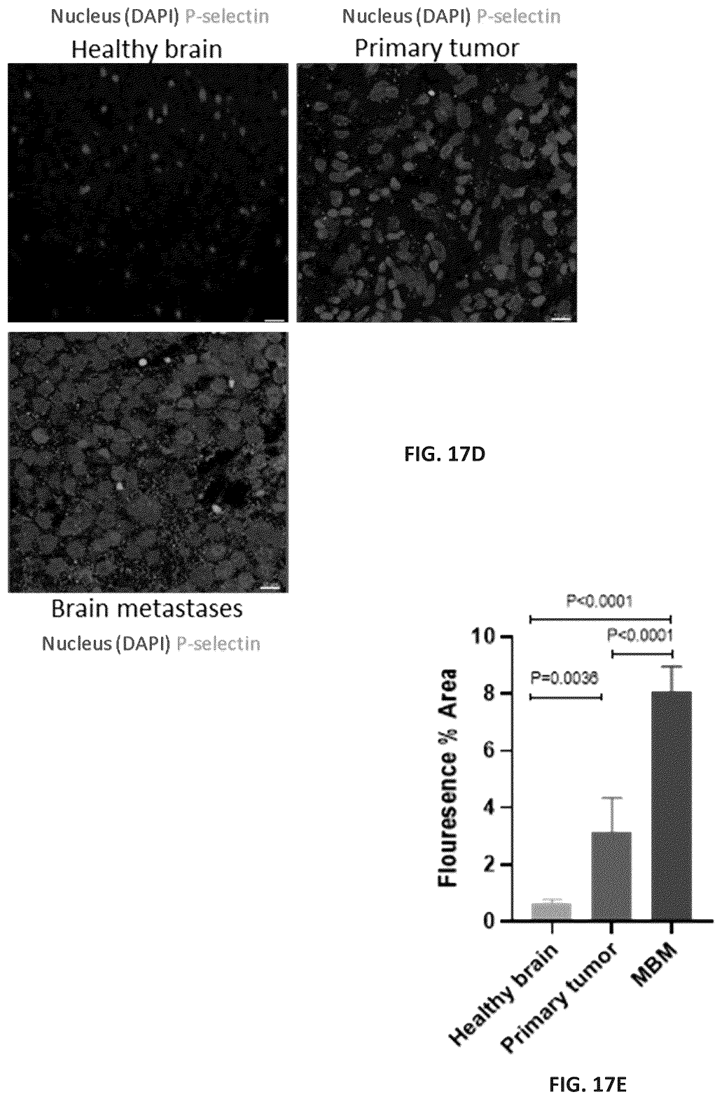

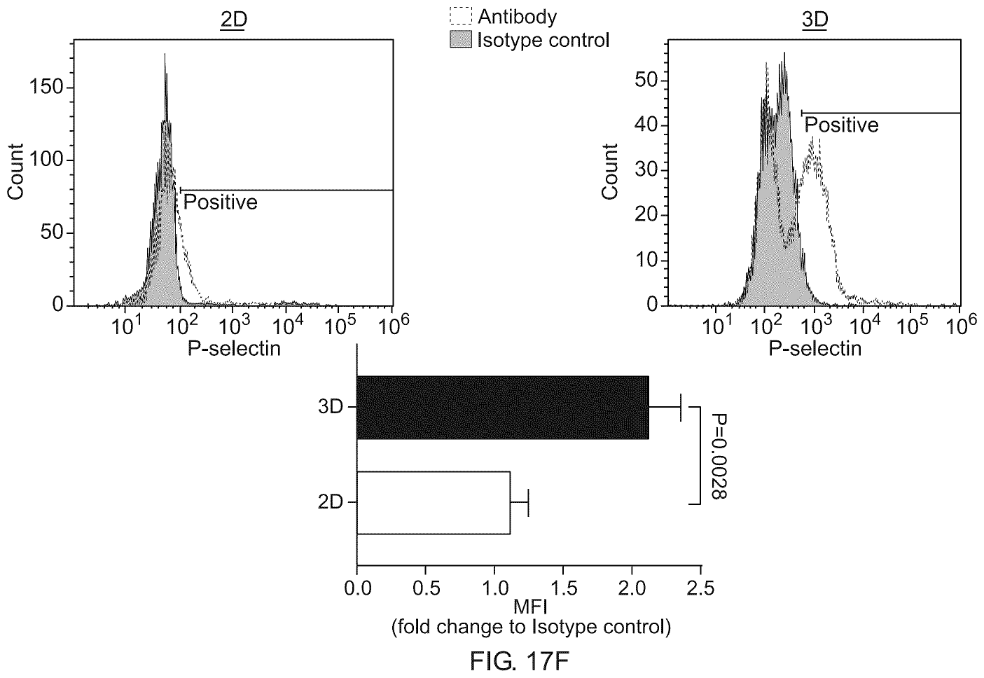

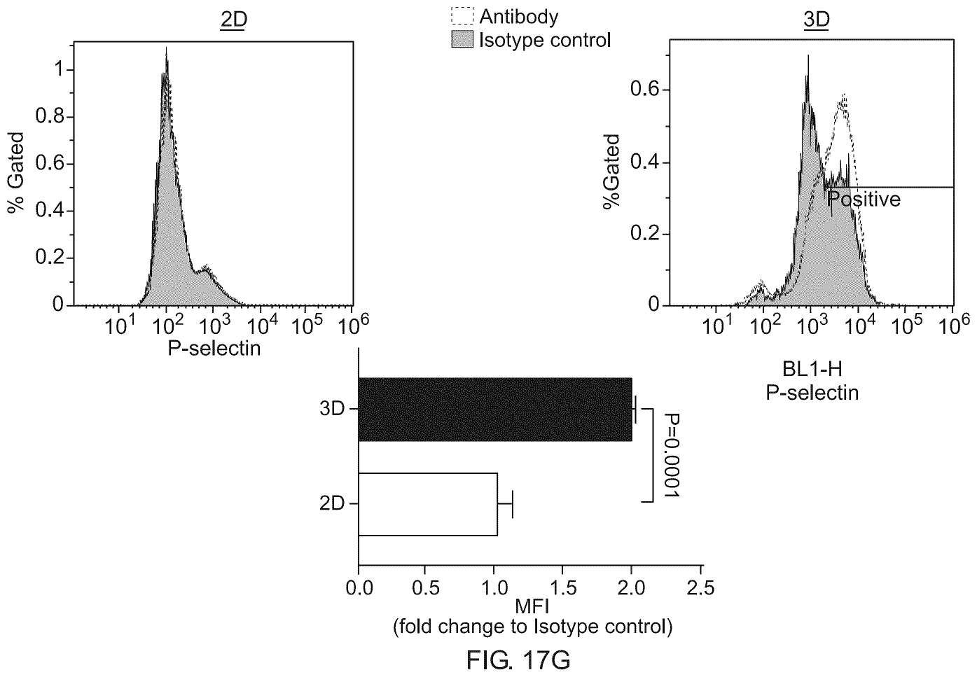

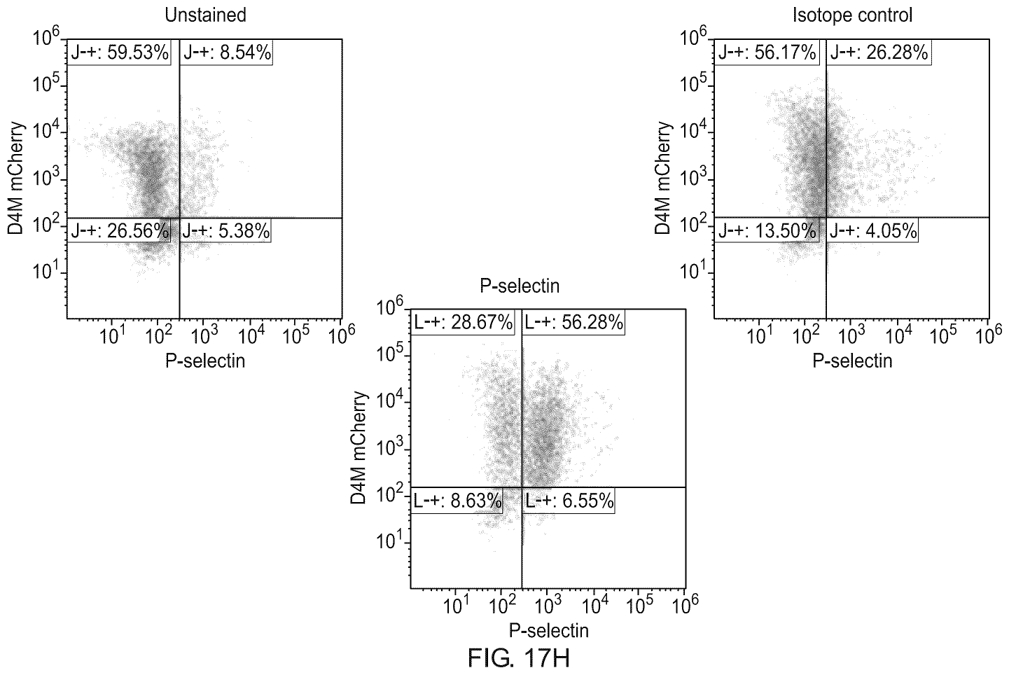

FIGS. 17A-I are representative images (FIGS. 17A and 17D) and respective quantifications (FIGS. 17C, 17D and 17F) of immunofluorescence staining of murine D4M.3A primary and brain metastases tumor tissues, healthy skin, and brain tissue samples (FIGS. 17A-C; Scale bar is 100 μm) and patient-derived specimens of melanoma primary and brain metastases tumor tissues, compared to a healthy brain (FIGS. 17D-E; scale bar is 10 μm). The nucleus was stained with DAPI (blue), and SELP was stained with Cy5-labeled antibody (cyan). Data represent mean±SD, at least 3 fields were images from each specimen, N=3. FIGS. 17F and 17G are flow cytometry analysis of SELP expression in murine melanoma D4M.3A (FIG. 17F) and human melanoma WM115 (FIG. 17G) cells grown in 2D or 3D culture models. Images are representative of 3 individual experiments. FIG. 17H are representative flow cytometry analysis of SELP expression in multicellular spheroids model comprised from mCherry-labeled D4M.3A cells and endothelial (bEnd.3) cells at a 1:2 ratio, respectively. FIG. 17H represent SELP expression in mCherry-labeled D4M.3A cells or bEnd3 cells (non-labeled), and FIG. 17I are histogram and quantification representing SELP expression from both cell lines in the 3D spheroids compared to cells grown in 2D cultures. Images are representative of 3 individual experiments. Statistical significance was determined using one-way ANOVA (p-value <0.05).

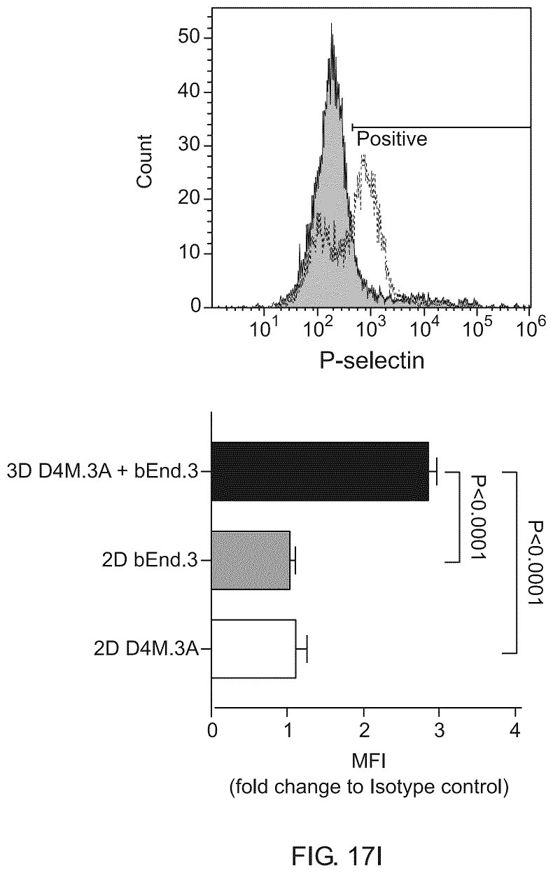

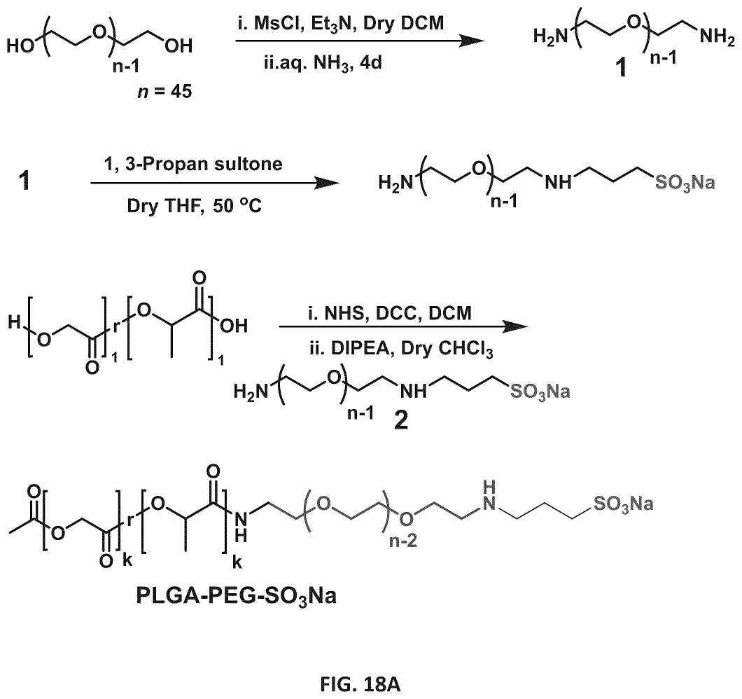

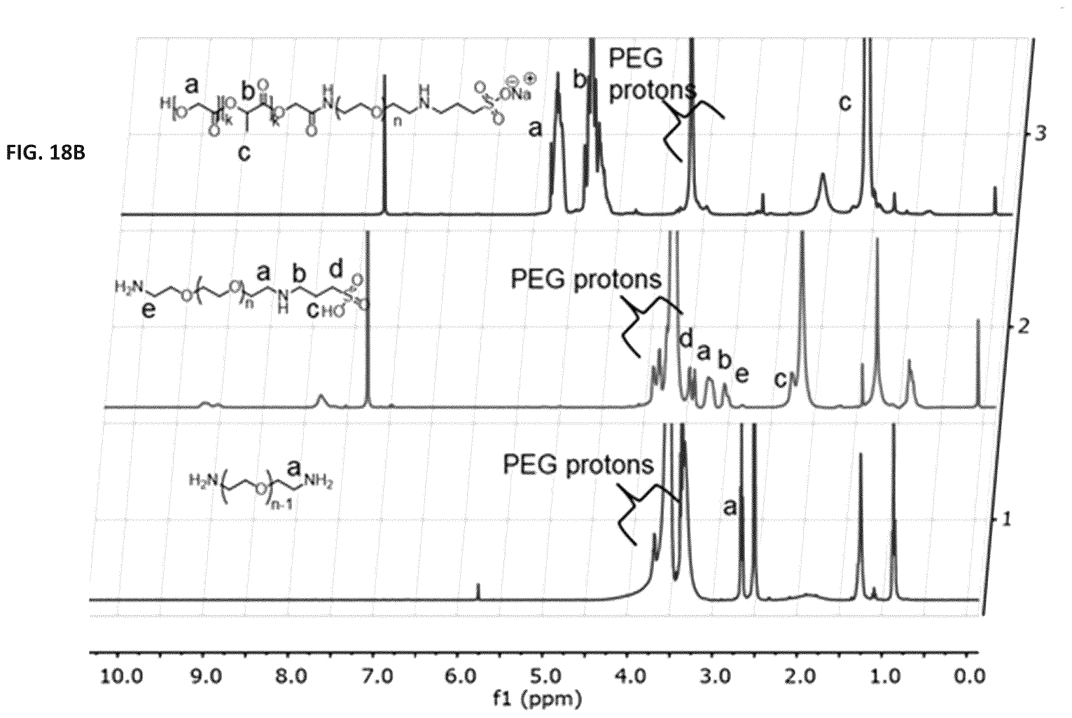

FIG. 18A is a synthetic scheme for the preparation of an exemplary sulfated PLGA-PEG according to some embodiments of the present invention, denoted PLGA-PEG-SO₃.

FIG. 18B is an overlay of ¹H NMR spectra of the exemplary PLGA-PEG-SO₃ (blue spectrum) and its precursors, PEG-SO₃ (polymer 2; green spectrum) and PEG diamine (polymer 1; red spectrum).

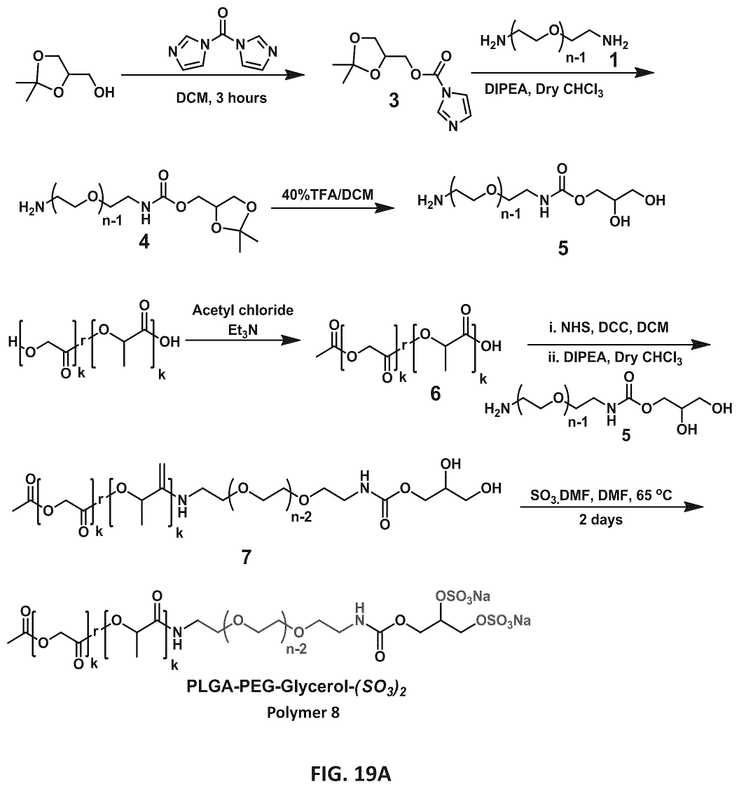

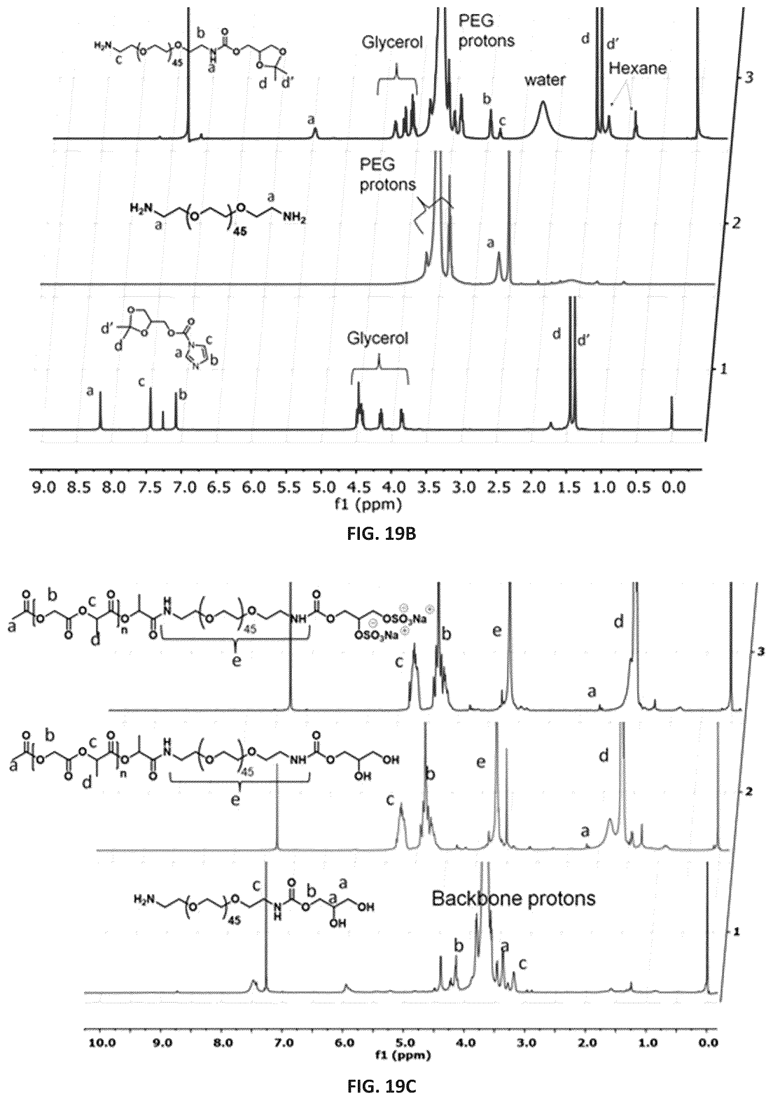

FIG. 19A is a synthetic scheme for the preparation of another sulfated PLGA-PEG according to exemplary embodiments of the present invention, denoted PLGA-PEG-Glycerol-(SO₃)₂ (Polymer 8).

FIG. 19B is an overlay of ¹H NMR spectra of solketal-based PEG amine (polymer 4; blue spectrum), PEG diamine (polymer 1; green spectrum) and solketal-based compound (compound 3; red spectrum).

FIG. 19C is an overlay of ¹H NMR spectra of an exemplary sulfated PLGA-PEG, PLGA-PEG-Glycerol-(SO₃)₂ (blue spectrum), and its precursors, polymer 7 (green spectrum) and polymer 5 (red spectrum).

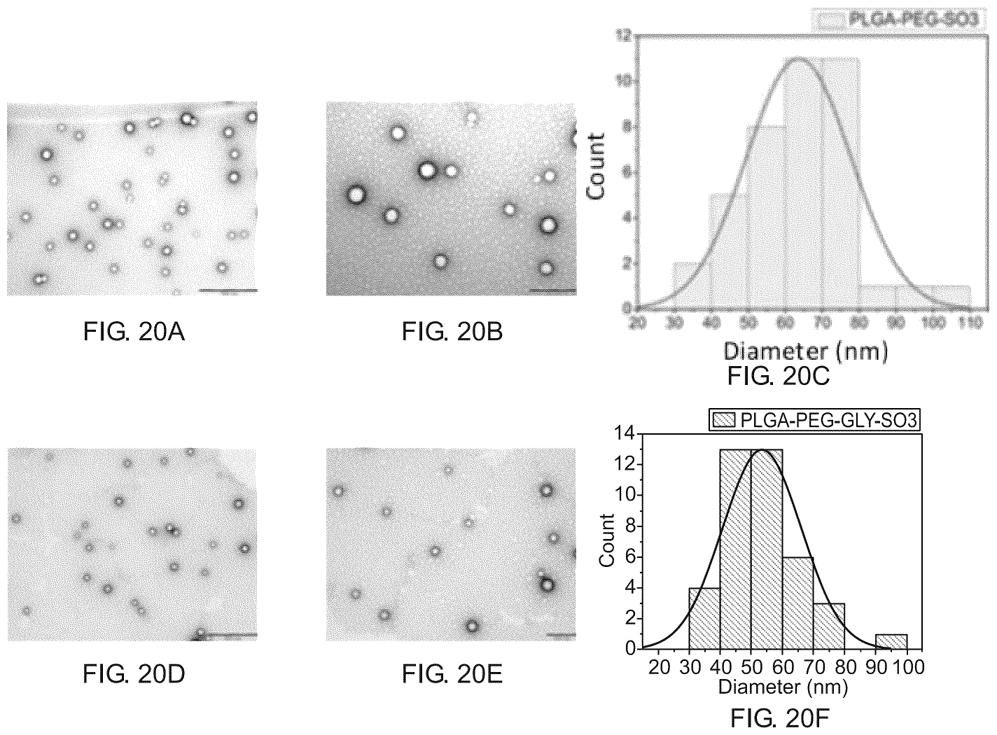

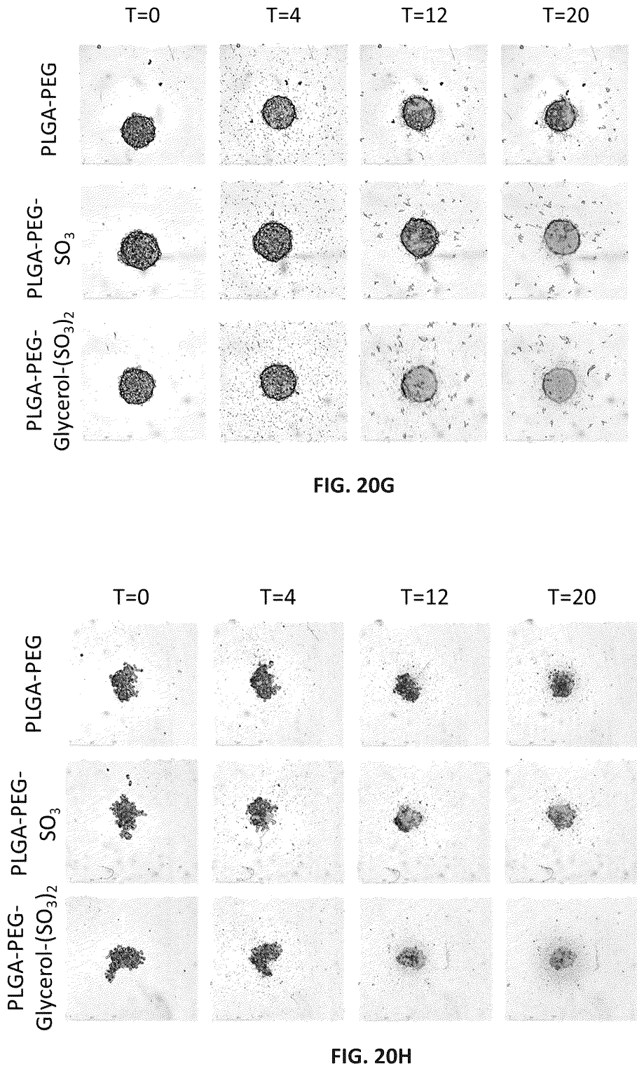

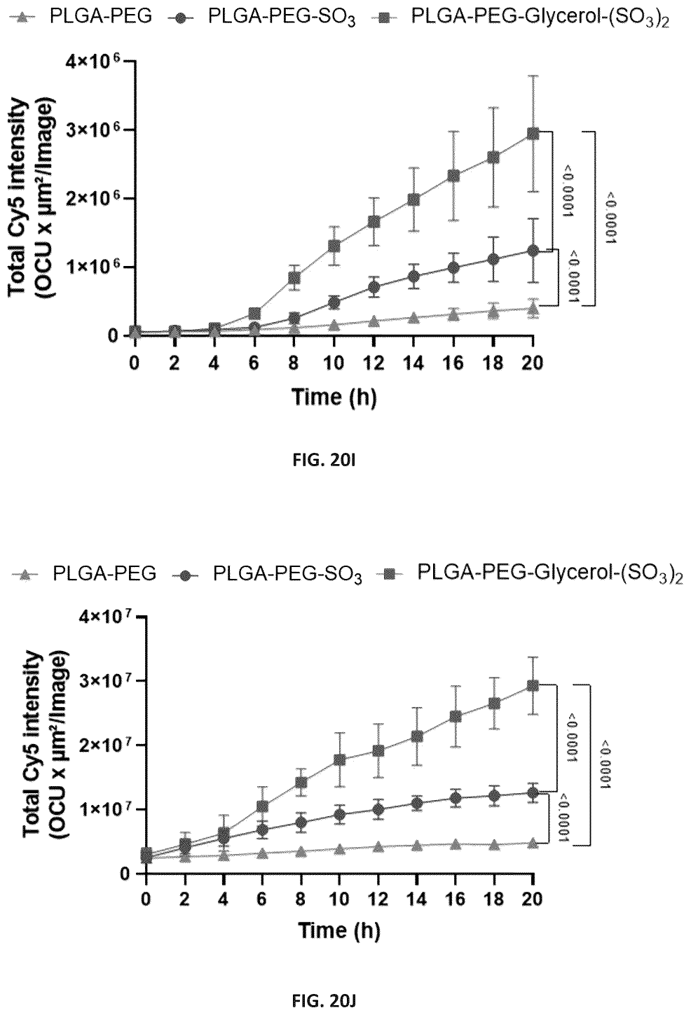

FIGS. 20A-F are representative TEM images and size distribution histograms of drug-loaded PLGA-PEG-SO₃(FIGS. 20A-C) and PLGA-PEG-Glycerol-(SO₃)₂ (FIGS. 20D-F) NPs. The size was calculated with ImageJ software, at least 40 NPs were measured for each group, the data correspond to mean±SD. Scale bar=500 nm (left image), 100 nm (right image).

FIGS. 20G and 20H are representative images of Cy5-labeled NPs internalization into D4M.3A (FIG. 20G) and WM115 (FIG. 20H) spheroids. Cells were seeded in ultra-low attachment 96 well plate and allowed to form spheroids for 72 hours. Followed, spheroids were treated with the NPs at Cy5-equivalent concentration (1 μM) for 20 hours. The images are representative of three independent experiments. Scale bar is 400 μm.

FIGS. 20I and 20J are comparative plots showing quantification of Cy5 intensity within the D4M.3A (FIG. 20I) and WM115 (FIG. 20J) spheroids. The quantification is representative of three independent experiments, data correspond to mean±SD of at least 12 spheroids per group. Images were acquired using the IncuCyte imaging system and the analysis was performed for the total Cy5 intensity measured within the spheroids (brightfield) boundaries. Statistical significance was determined using a two-way ANOVA test with multiple comparisons adjustment.

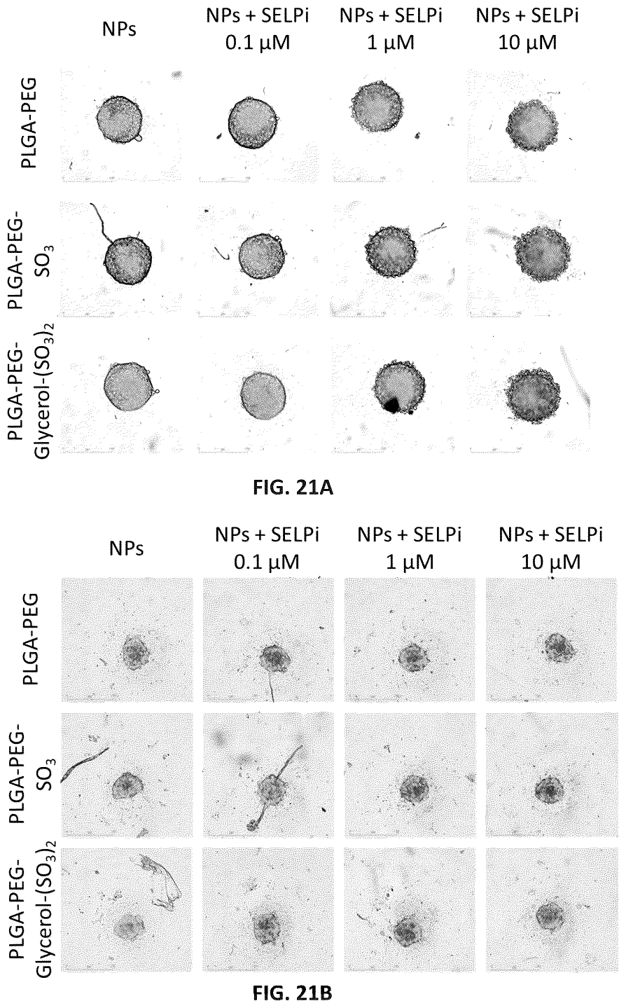

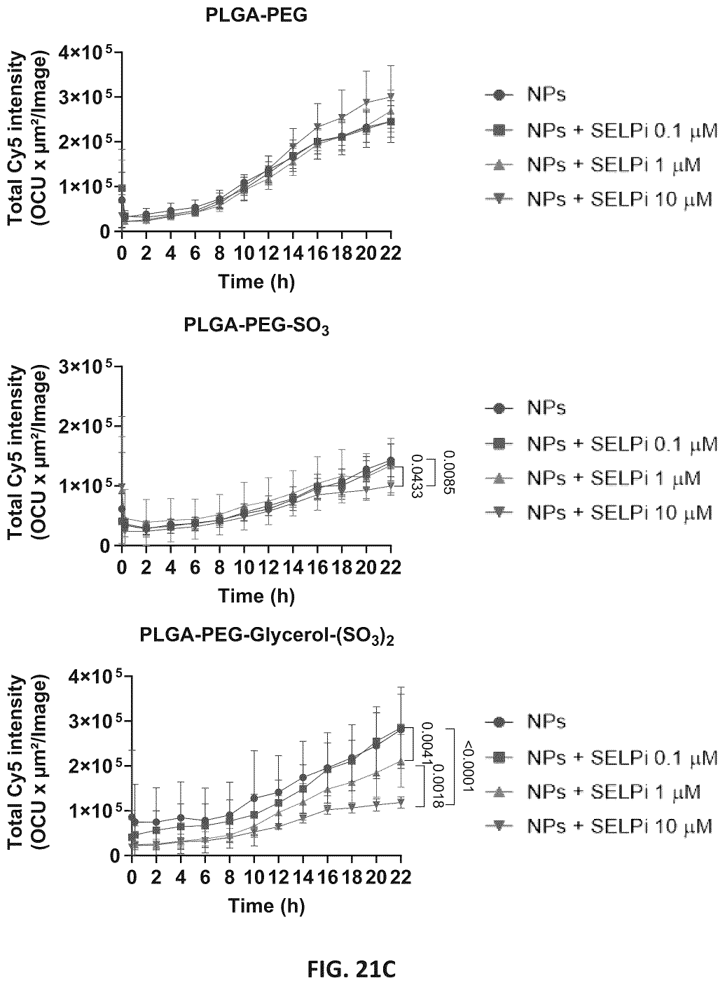

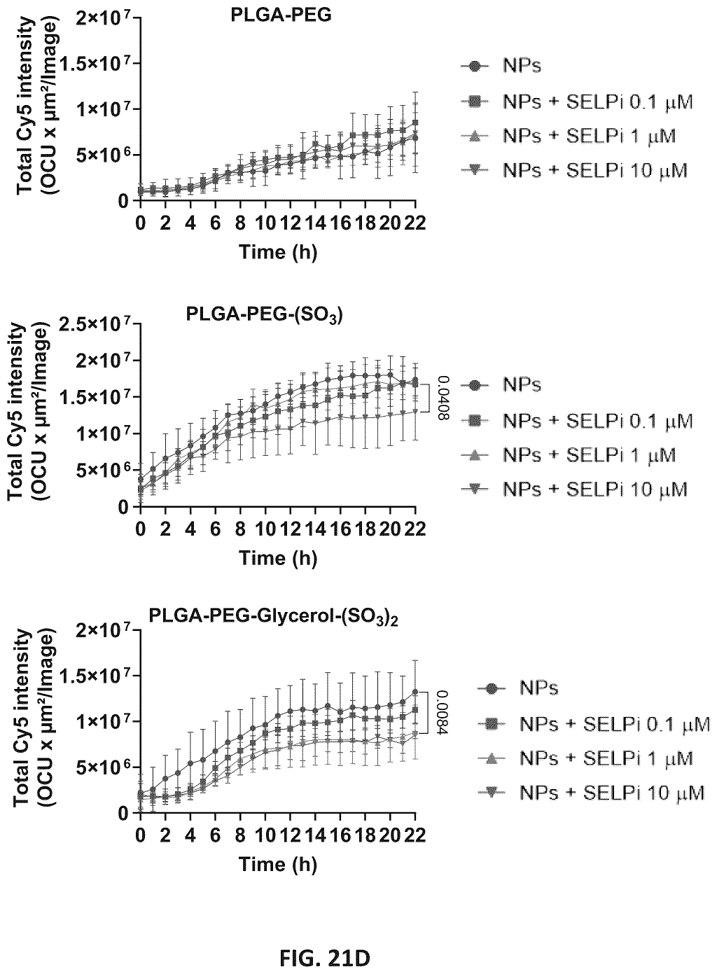

FIGS. 21A-B are representative images of Cy5-labeled NPs internalization into D4M.3A (FIG. 21A) and WM115 (FIG. 21B) spheroids 1 hour after treatments with serial dilutions of SELP inhibitor (SELPi). Spheroids were prepared as described in FIG. 20G.

FIGS. 21C-D are comparative plots showing quantification of Cy5 intensity within the D4M.3A (FIG. 21C) and WM115 (FIG. 21D) spheroids, at 3 different SELPi concentrations. The quantification is representative of three independent experiments, data correspond to mean±SD of at least 12 spheroids per group. Images were acquired using the IncuCyte imaging system and the analysis was performed for the total Cy5 intensity measured within the spheroids (brightfield) boundaries. Statistical significance was determined using a two-way ANOVA test with multiple comparisons adjustment.

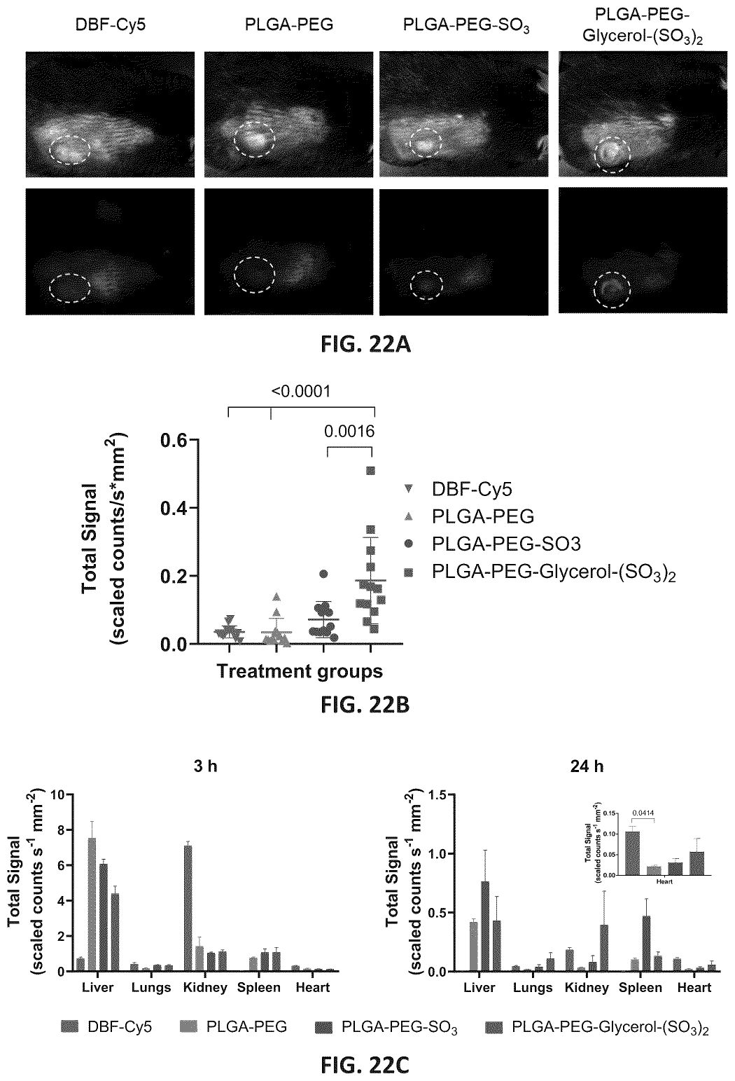

FIG. 22A presents representative images of D4M.3A tumor accumulation of Cy5-labeled targeted NPs, non-targeted NPs, or free DBF-Cy5, 2 hours after IV administration. N=6-7 mice per group and the experiment was repeated twice.

FIG. 22B presents comparative plots showing quantification of individual Cy5-intensity values detected from each tumor. The data signify mean±SEM. Statistical significance was calculated using a one-way ANOVA test with multiple comparisons adjustment.

FIG. 22C presents bar graphs showing bio-distribution following 3 hours (left graph) and 24 hours (right graph) after the IV administration as described in FIG. 22A. The organ's fluorescent signal was normalized to its surface area (mm²). N=3 mice per group, two-way ANOVA was used for statistical analysis.

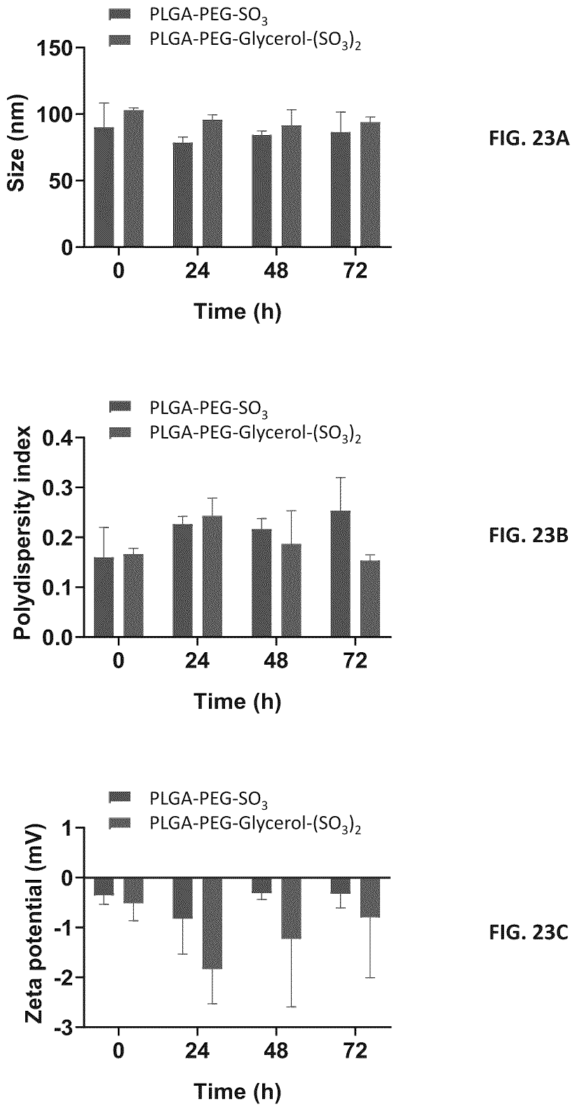

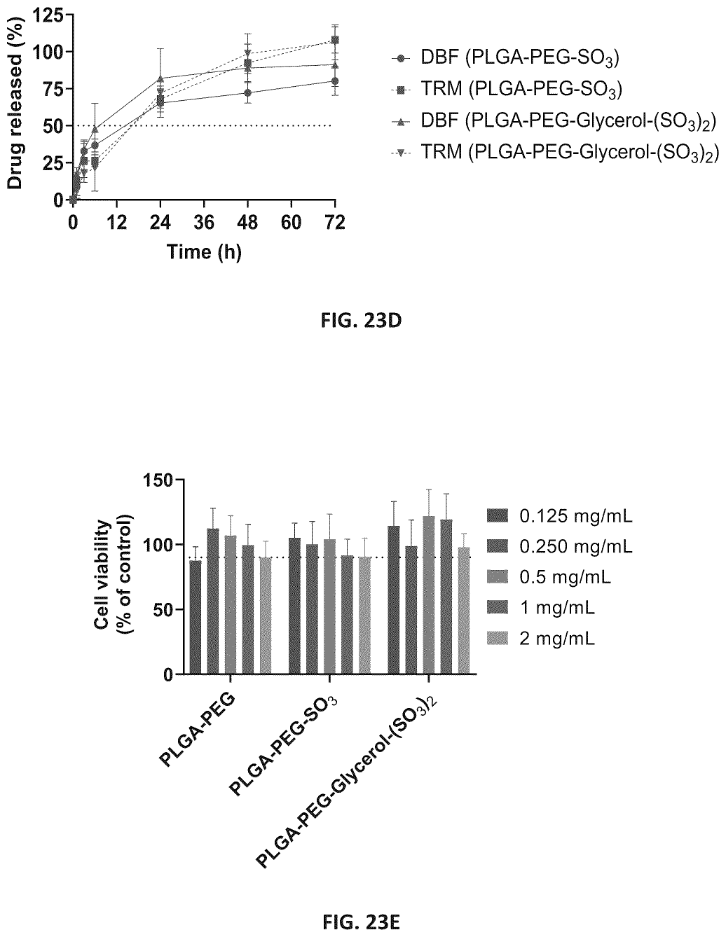

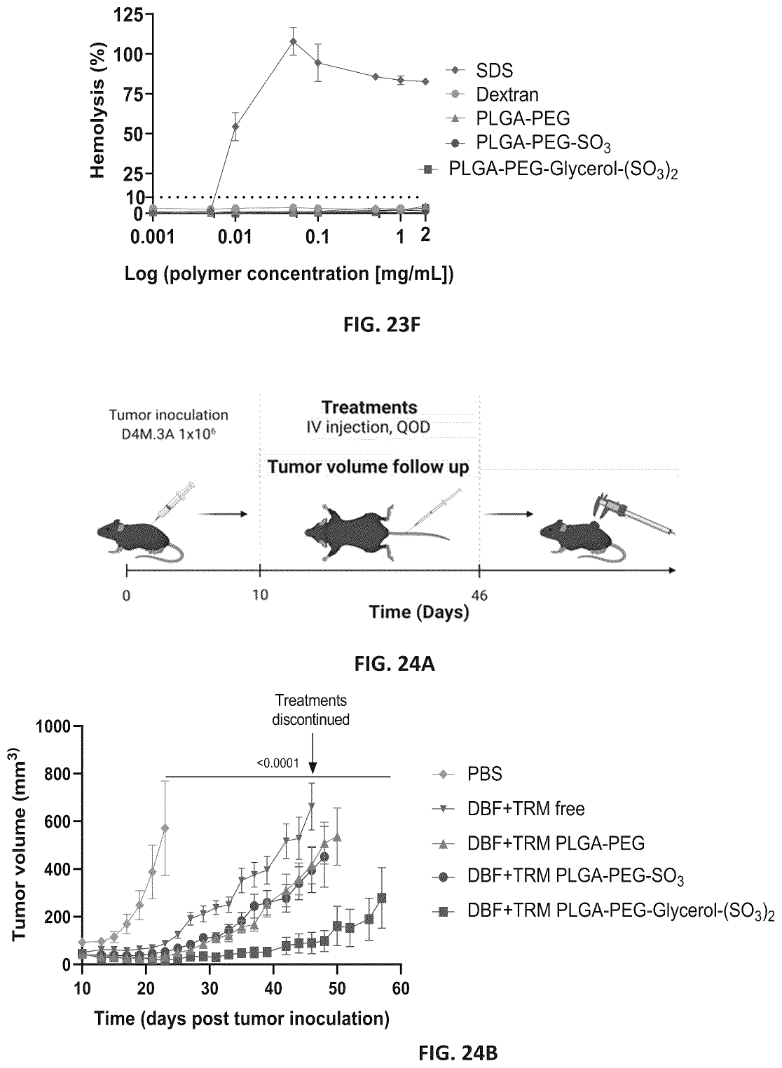

FIGS. 23A-C present bar graphs showing stability measurements by DLS: hydrodynamic size (FIG. 23A), dispersity index (FIG. 23B), and zeta potential (FIG. 23C). The NPs were suspended in PBS and gently stirred for 72 hours at 37° C.

FIG. 23D presents comparative plots showing release profiles of DBF and TRM from targeted NPs. The NPs were aliquoted into 200 μL dialysis tubes and incubated in 1 Liter PBS at 37° C. At predetermined time points, 200 μL of targeted NPs were collected, lyophilized, and analyzed for drug content. The data represent the average of three independent repeats, and it signifies mean±SD.

FIG. 23E is a bar graph showing NIH/3T3 fibroblast cell viability after incubation with serial dilution of SELP-targeted NPs and non-targeted NPs for 72 hours, as indicated. The data represent the average of three independent repeats, and it signifies mean±SD.

FIG. 23F presents a comparative plot, showing dose-dependent hemolysis % in the presence of SELP-targeted NPs or non-targeted NPs. Red blood cells (RBC) were treated with increasing concentrations of SELP-targeted NPs, non-targeted NPs, dextran (negative control), or sodium dodecyl sulfate (SDS, positive control) for 1 hour, and the percentage of hemoglobin released was measured using a spectrophotometer.

FIG. 24A is a schematic presentation of the study protocol, including timeline (days) of tumor inoculation, treatments, and follow-up, performed in order to evaluate the anti-tumor activity of the targeted nanoparticles. C57BL6 mice were inoculated with D4M.3A cells and were allowed to establish tumors until the average tumor volume was 50 mm³ (10 days). The mice were then randomized into groups and were IV administered with the combination of 1 mg/kg DBF and 0.1 mg/kg TRM as free drugs, exemplary targeted (PLGA-PEG-SO₃ or PLGA-PEG-Glycerol-(SO₃)₂), or non-targeted (PLGA-PEG) drug-loaded nanoparticles. The mice were treated QOD until the first group reached the endpoint of tumor volume above 1000 mm³.

FIG. 24B presents comparative plots showing tumor volume growth curve in the different treatment groups tested in the study described in FIG. 24A. The data show that the exemplary drug-loaded nanoparticles prolong survival of the mice compared to the control and free drugs, and that treatment with PLGA-PEG-Glycerol-(SO₃)₂ nanoparticles reduces tumor volume compared to the non-targeted and PLGA-PEG-SO₃ nanoparticles. P values correspond to tumor volume of the treatment groups compared to PBS at day 23.

FIGS. 24C-E present quantification data of individual tumor volumes of the tested treatment groups at the particular time points: at day 46 (FIG. 24C), day 48 (FIG. 24D), and day 57 (FIG. 24E). The data show that the drug-loaded PLGA nanoparticles reduce tumor size compared to the free drugs and non-targeted nanoparticles. Statistical significance was calculated for groups that did not reach the first endpoint. Data are shown as mean±SEM. Statistical significance was determined using two-sided repeated-measures ANOVA (p-value <0.05). N=5 mice for the PBS group and N=8-10 mice for the remaining groups.

FIG. 24F presents comparative plots presenting bodyweight change curve following different treatment courses. The body weight is expressed as a percent change from the day of treatment initiation, as a safety evaluation. No substantial change in bodyweight was observed. Data represent mean±SEM.

FIG. 24G presents a Kaplan-Meier survival curve for mice inoculated with 1×10⁶ D4M.3A cells. The statistical significance shown in the graph corresponds to PLGA-PEG-Glycerol-(SO₃)₂ NPs compared with free drugs. Additionally, P=0.0006 for PLGA-PEG-Glycerol-(SO₃)₂ NPs compared with PLGA-PEG NPs. P<0.0001 for all groups compared with PBS. P values were determined using a log-rank test. The data show that both the drug-loaded PLGA nanoparticles prolong survival of the mice compared to the control, free drugs, and non-targeted (PLGA-PEG) nanoparticles.

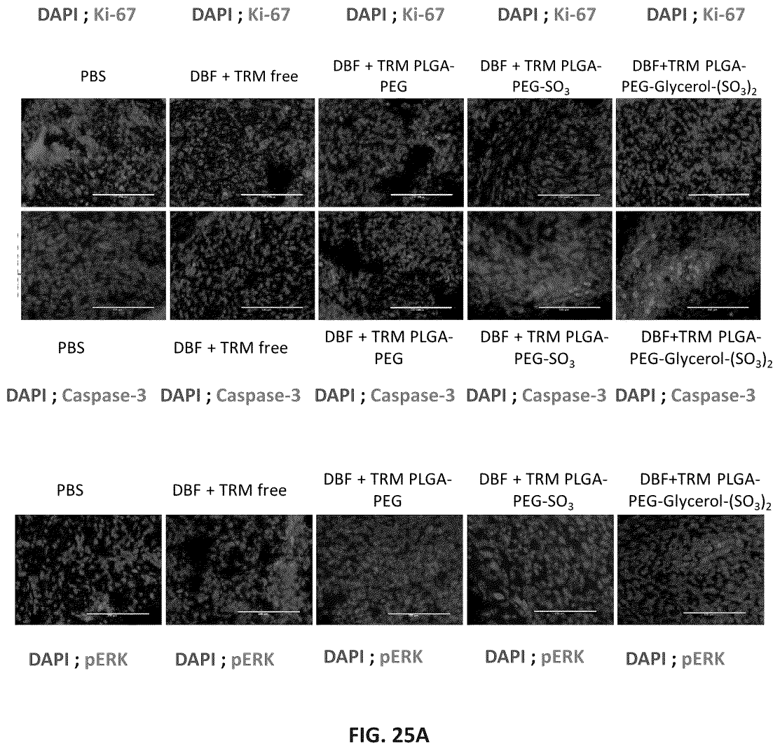

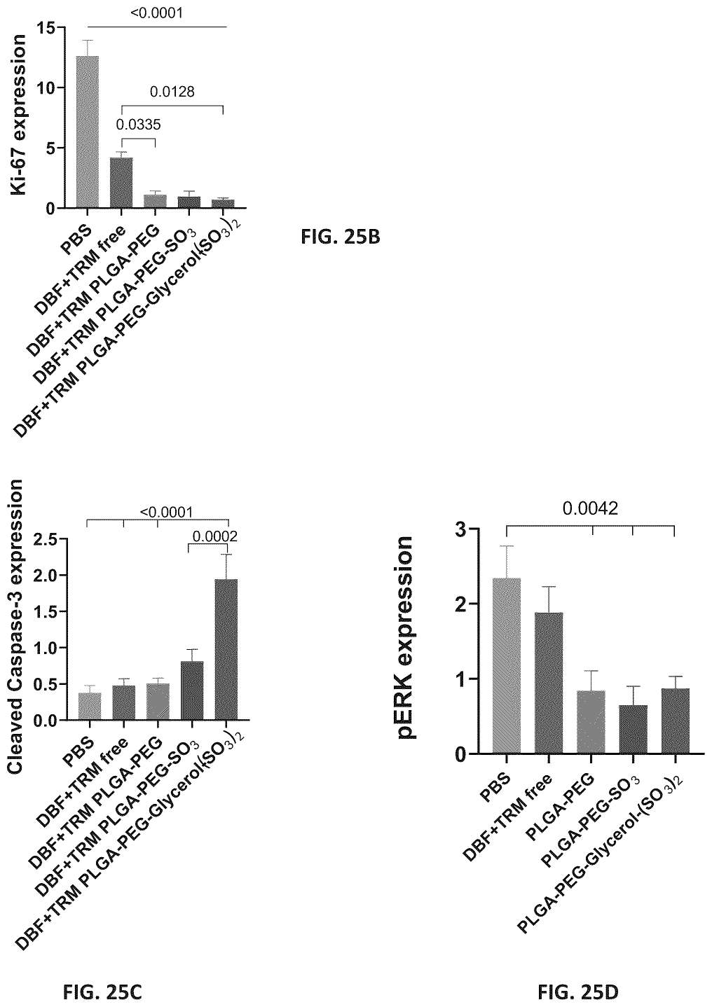

FIGS. 25A-D present representative immunofluorescence staining (FIG. 25A) of D4M.3A tumors treated with free drugs, non-targeted nanoparticles, or targeted nanoparticles, as described in FIG. 24A, and the quantification of the immunofluorescence staining (FIGS. 25B-D). Mice were treated with DBF and TRM as free drugs or drug-loaded targeted (PLGA-PEG-SO₃ or PLGA-PEG-Glycerol-(SO₃)₂), or non-targeted (PLGA-PEG) NPs. The tumors were collected 16 hours after the 9^{th} treatment and stained for proliferative cells (Ki-67), apoptotic cells (Cleaved caspase-3) and phosphorylated ERK 1/2. Tumor cell nucleus are shown in blue (DAPI) and positive immunostaining is shown in magenta. N=3 mice per group, at least 6 images per mouse. Statistical significance was determined using a one-way ANOVA test with multiple comparisons adjustment. Scale bars are 100 μm. Data show mean±SEM.

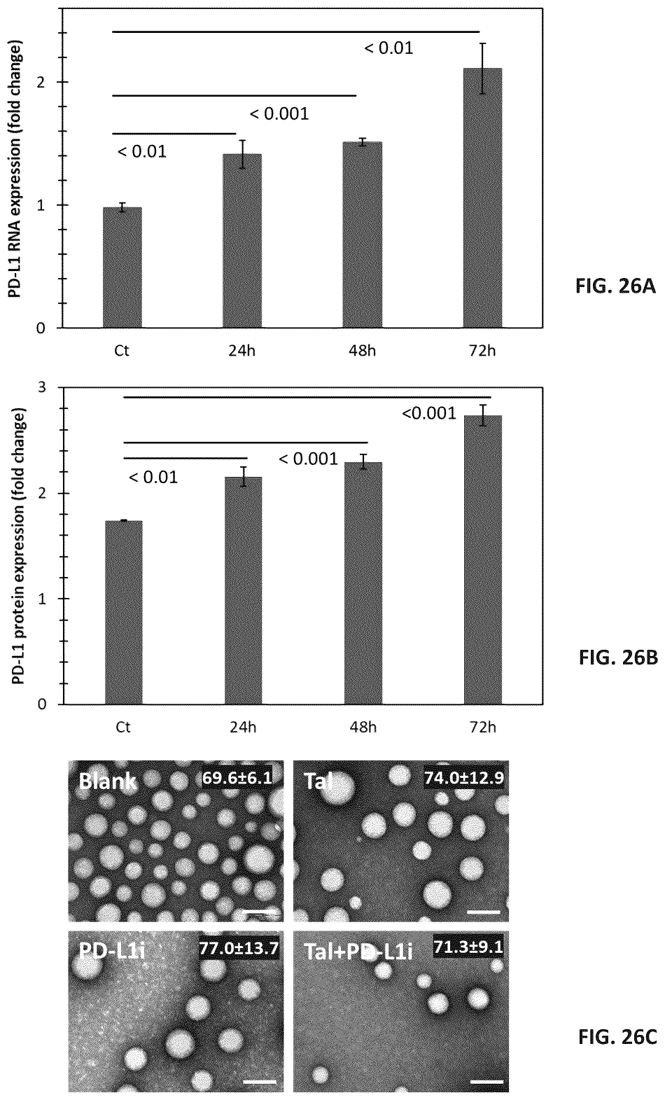

FIGS. 26A-B are bar graphs of PD-L1 RNA (FIG. 26A) and PD-L1 protein expression (FIG. 26B) in EMT6 murine mammary carcinoma cells following treatment with 10 nM talazoparib over 72 hours incubation.

FIG. 26C presents representative TEM images of unloaded (blank)-, talazoparib-loaded-, PD-L1i-loaded-, and talazoparib+PD-L1i-loaded-PLGA-PEG-Glycerol-(SO₃)₂ NPs.

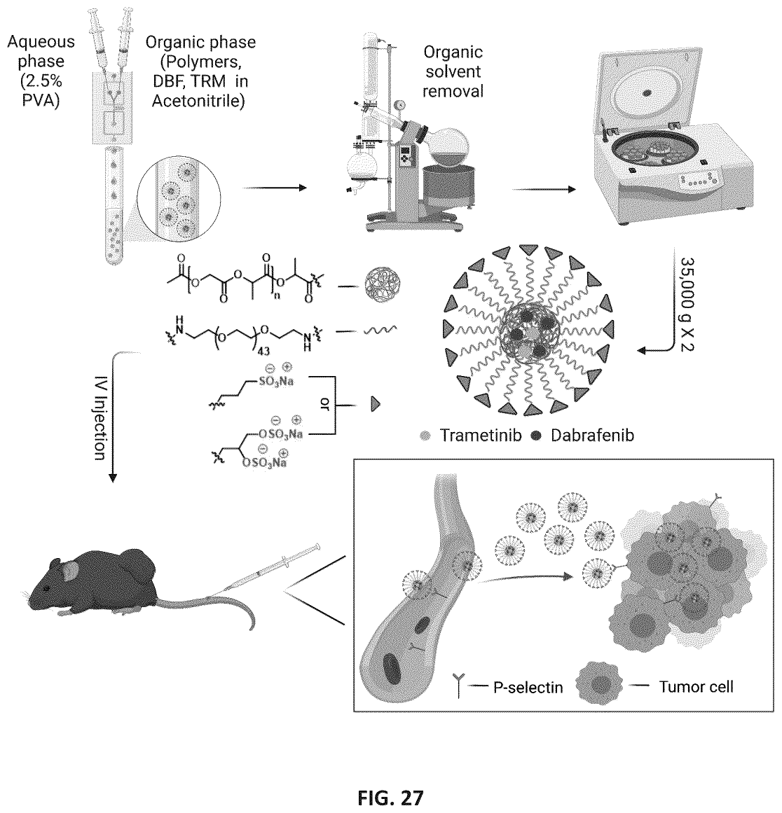

FIG. 27 is a schematic illustration of the preparation and IV-administration of exemplary sulfated PLGA-PEG-SO₃ and/or PLGA-PEG-Glycerol-(SO₃)₂NPs.

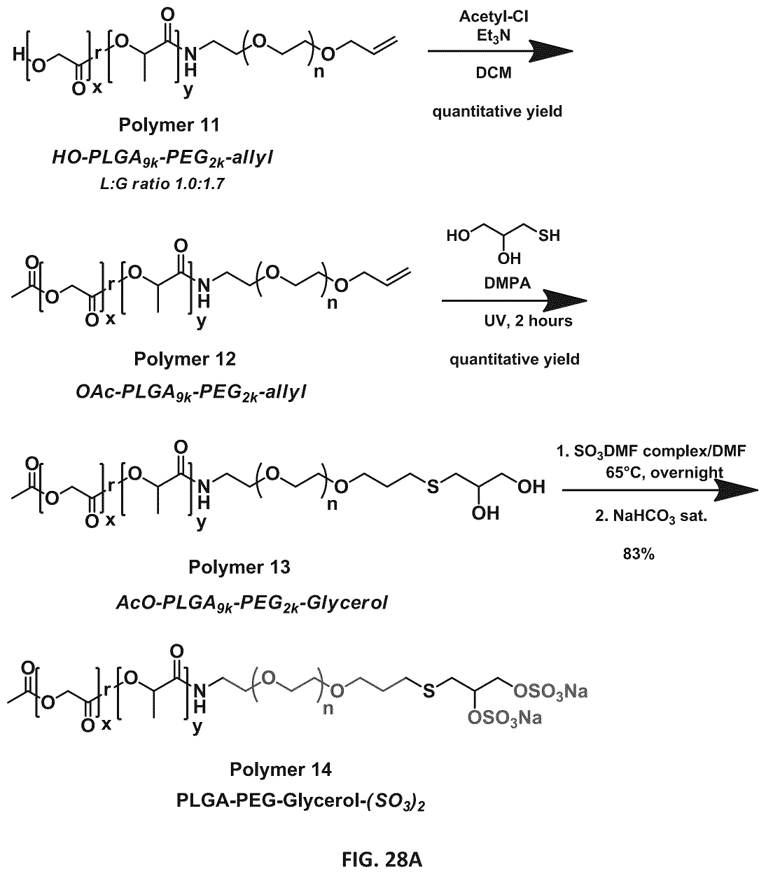

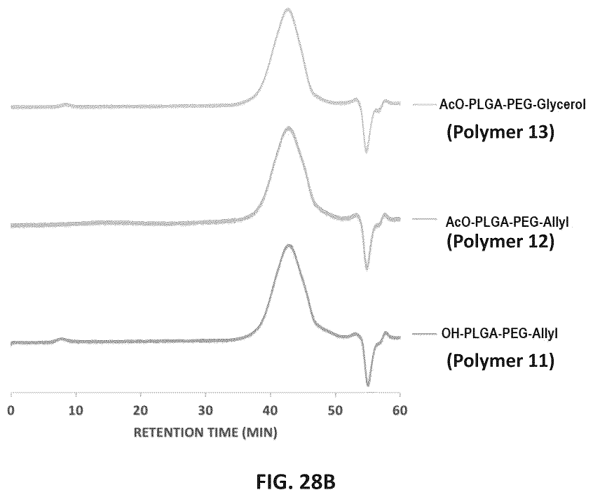

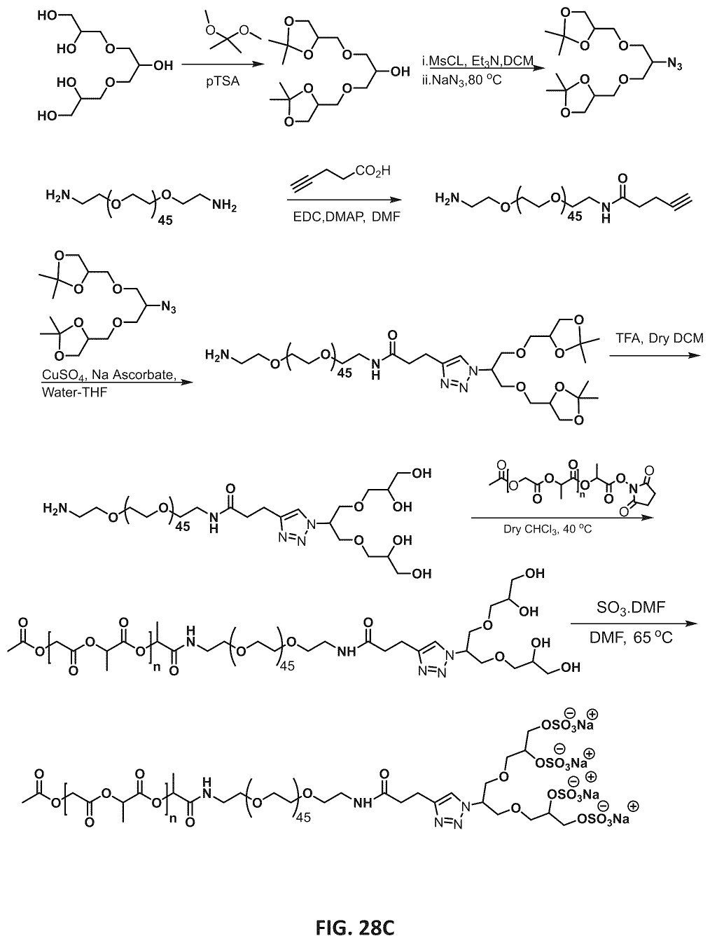

FIG. 28A is a synthetic scheme for the preparation of an exemplary sulfated PLGA-PEG according to some embodiments of the present invention, denoted PLGA-PEG-Glycerol-(SO₃)₂ (Polymer 14).

FIG. 28B is a SEC analysis showing traces overlay of commercial HO-PLGA-PEG-allyl (Polymer 11) alongside the modified amphiphiles AcO-PLGA-PEG-allyl (polymer 12) and AcO-PLGA-PEG-glycerol (Polymer 13), as indicated.

FIG. 28C is a synthetic scheme for the preparation of an exemplary sulfated PLGA-PEG according to some embodiments of the present invention, denoted PLGA-PEG-Glycerol-(SO₃)₄.

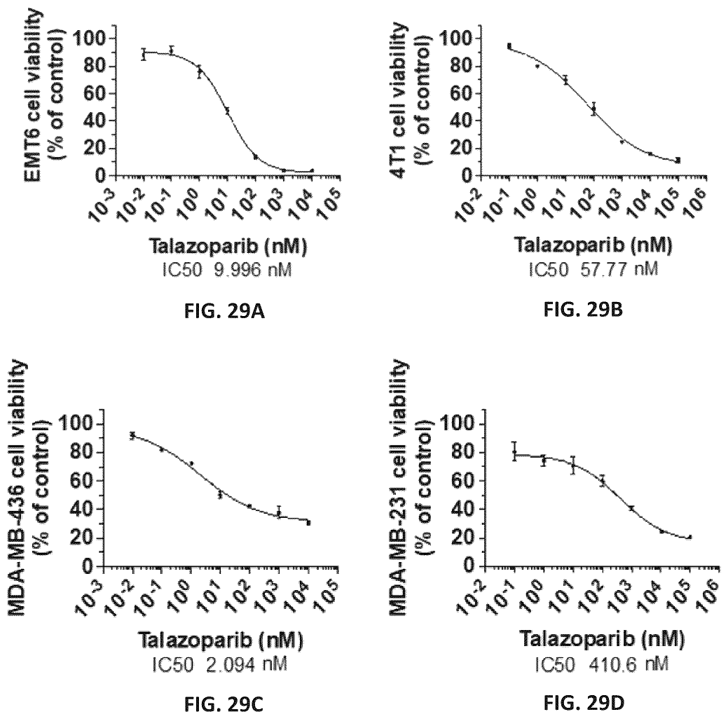

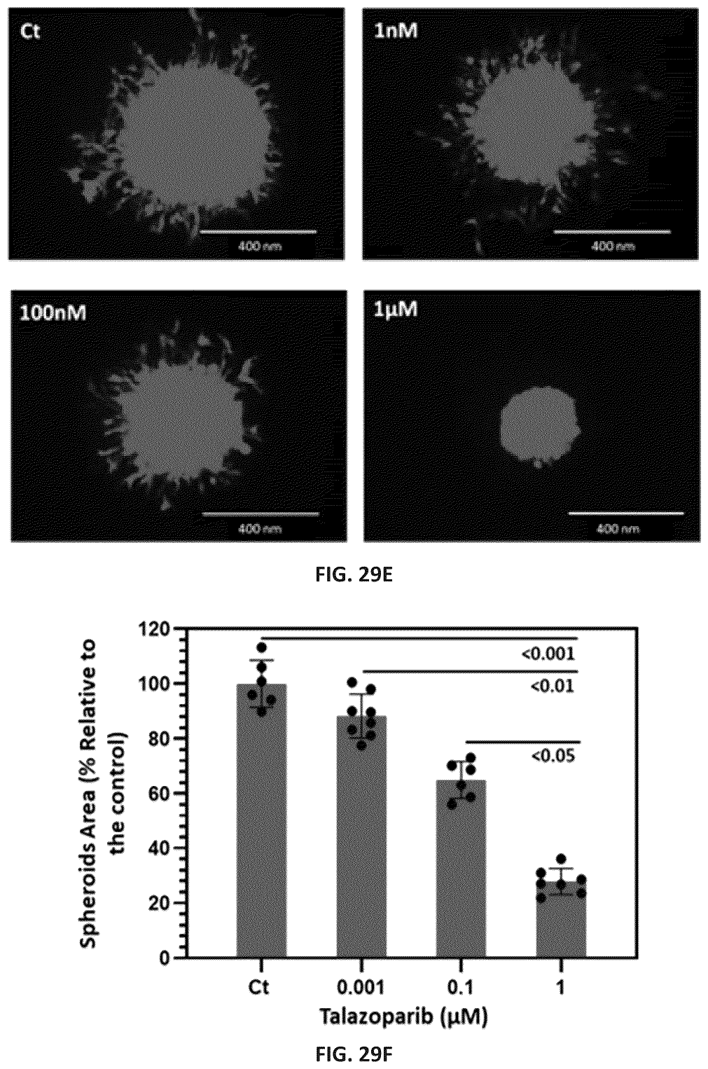

FIGS. 29A-D present graphs showing breast cancer (BC) cell viability as a function of inhibitor (talazoparib) concentration, demonstrating inhibition of BC cell proliferation and 3D spheroid sprouting following incubation with a serial dilution of talazoparib for 72 hours. The tested cells included EMT6 (FIG. 29A), 4T1 (FIG. 29B), MDA-MB-436 (FIG. 29C) and MDA-MB-231 (FIG. 29D). Data represent mean±SD (N=3). IC₅₀ values were calculated using non fit linear regression and four parameters variable slope.

FIGS. 29E-F are representative images of dose-dependent effect of free talazoparib (0, 1 nM, 100 nM and 1 μM, as indicated) on the sprouting of mCherry-labeled EMT6 3D spheroids (FIG. 29E) and quantification graph of mCherry % fluorescent area of the mCherry-labelled EMT6 spheroids. Data represent mean±SD (N=1, n=6).

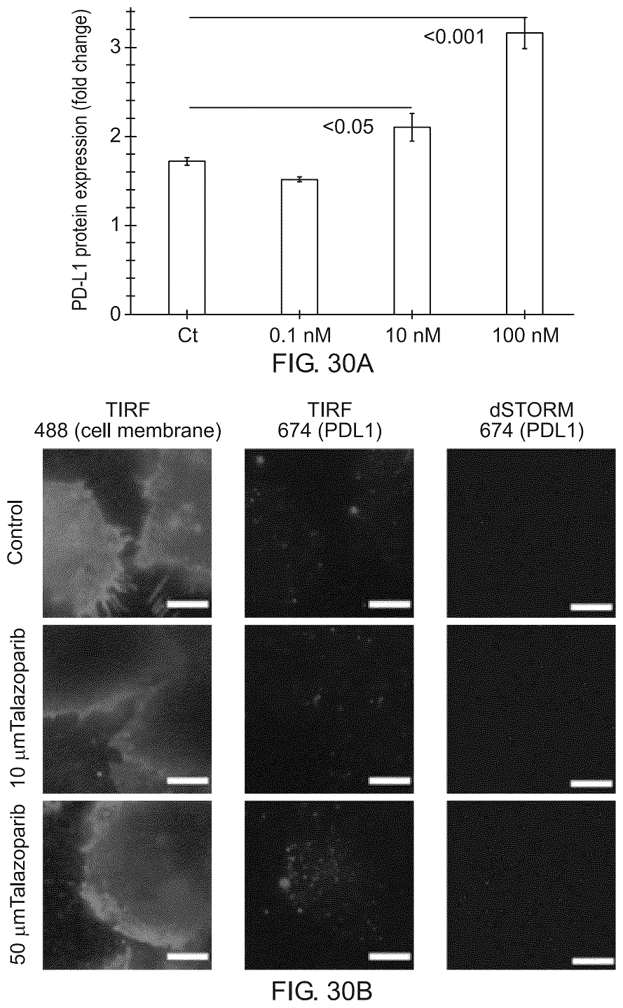

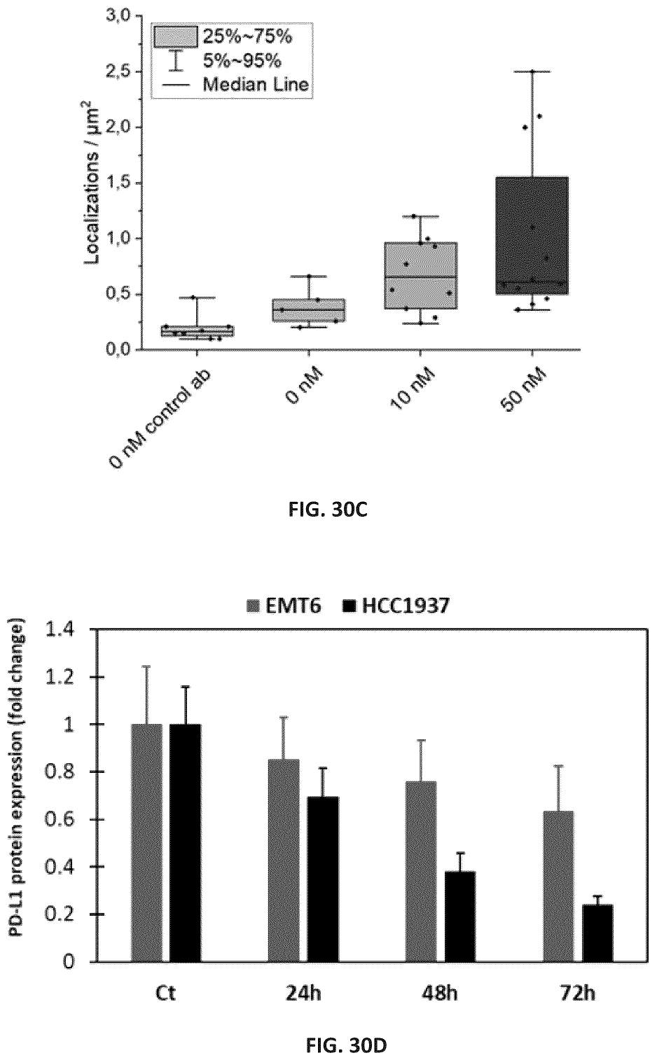

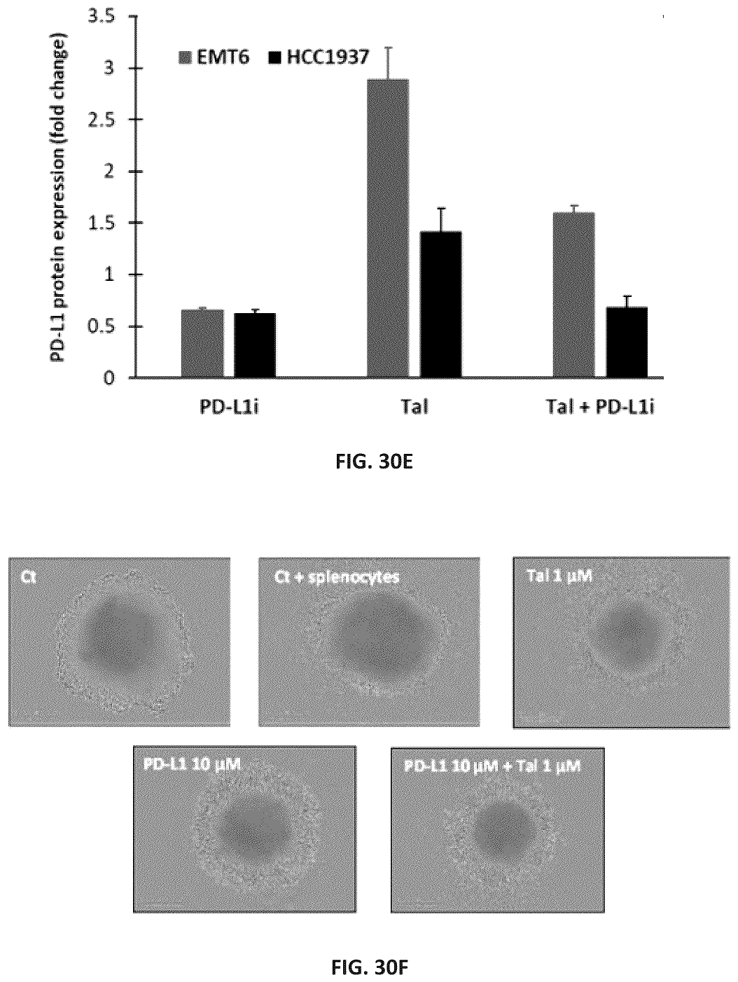

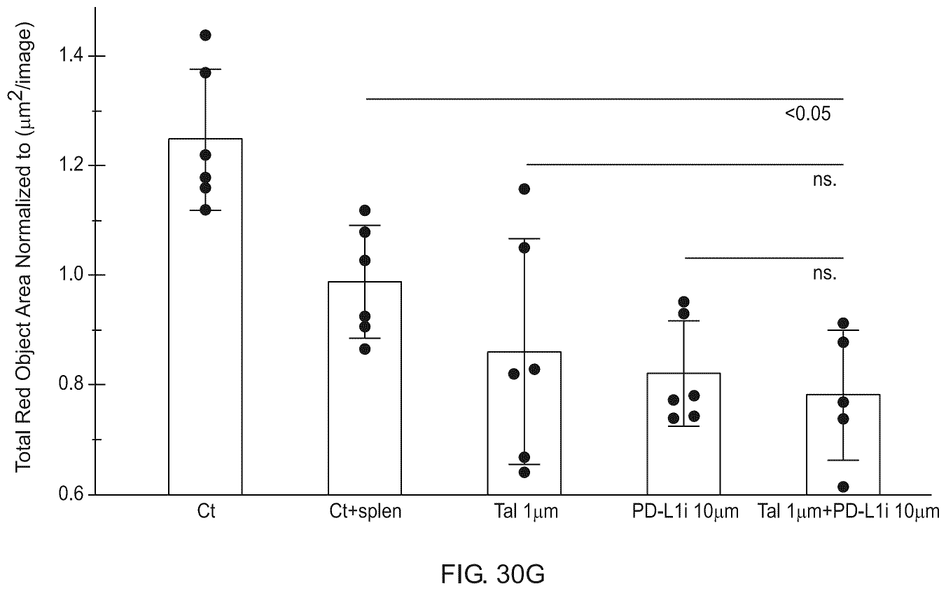

FIG. 30A is a bar graph of PD-L1 protein expression in EMT6 murine mammary carcinoma cells following treatment with increasing talazoparib concentrations over 72 hours incubation, as obtained from flow cytometry analysis.

FIG. 30B are dSTORM imaging showing PD-L1 localizations per μm² in EMT6 cells with higher concentrations of talazoparib. Blue: TIRF microscopy cell membrane; Red: TIRF microscopy PD-L1 and dSTORM microscopy PD-L1 representative images, as indicated. Data represent mean±SD (N=3).

FIG. 30C is a graph showing dSTORM localization per cell following 48 hour-treatment with talazoparib, as obtained from the dSTORM analysis of FIG. 30B.

FIGS. 30D-E are bar graphs showing flow cytometry analysis of PD-L1 expression in EMT6 (red) or HCC1937 (black) following treatment with PD-L1 small molecule (10 μM) (SM56 for murine cell line and SM69 for human cell line) at 24 hours and 48 hours (FIG. 30D); and under combination with talazoparib (10 nM) after 48 hours of incubation (FIG. 30E).

FIG. 30F are representative images of EMT6 spheroids co-cultured with activated splenocytes, at 1:100 ratio, respectively, following treatments with talazoparib and SM56, separately or combined.

FIG. 30G is a bar graph showing quantification of mCherry area of the analyses of FIG. 30F. Data represent mean±SD (N=3, n=10).

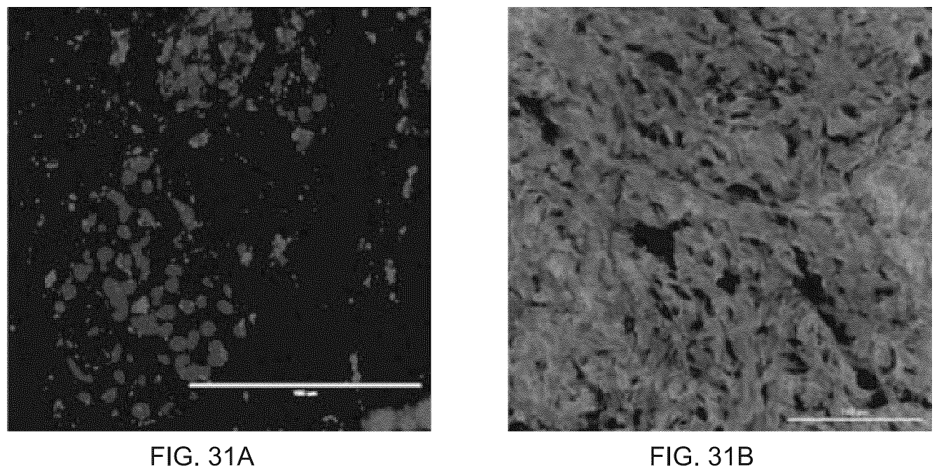

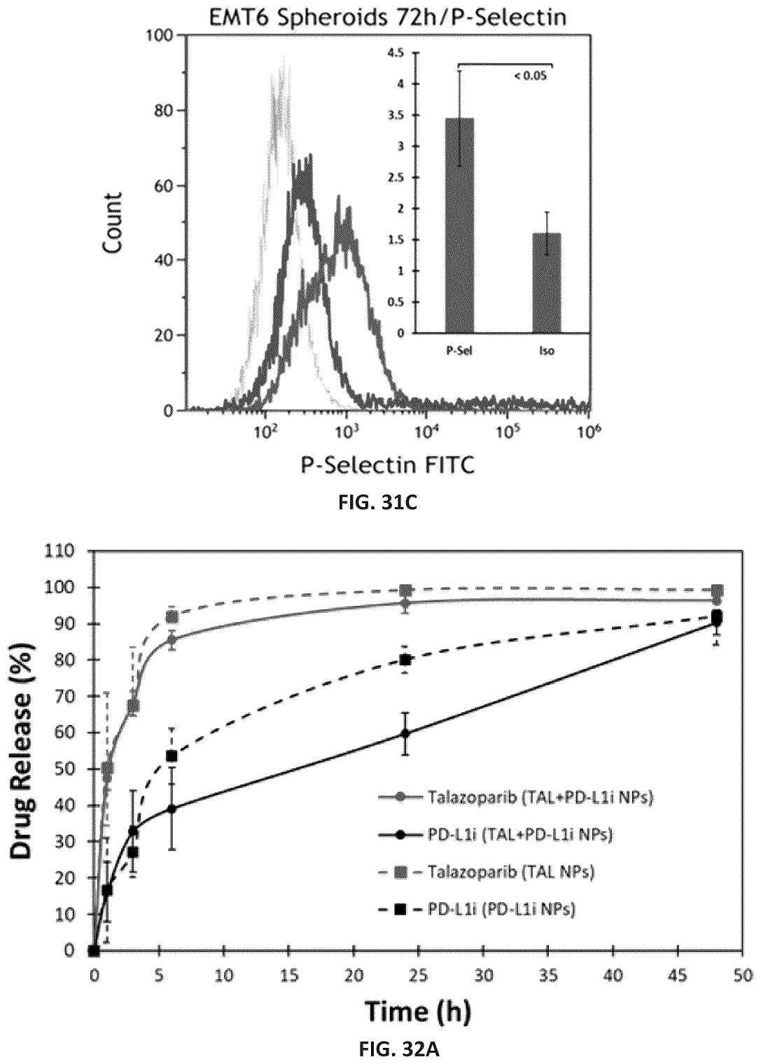

FIGS. 31A-B are representative images showing SELP is expressed in BRCA-mutated EMT6 primary (FIG. 31B) and brain metastases (FIG. 31A). Nuclei are stained with DAPI in blue and SELP in red.

FIG. 31C is a FACS analysis of the 3D spheroids and 2D cultured cells of FIGS. 31A-B, indicating the expression of SELP.

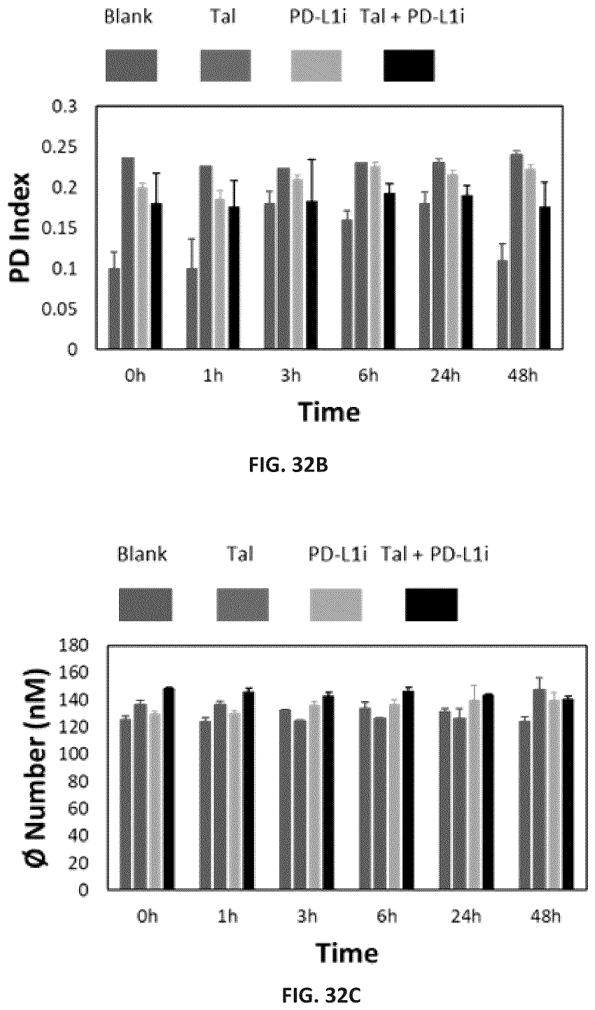

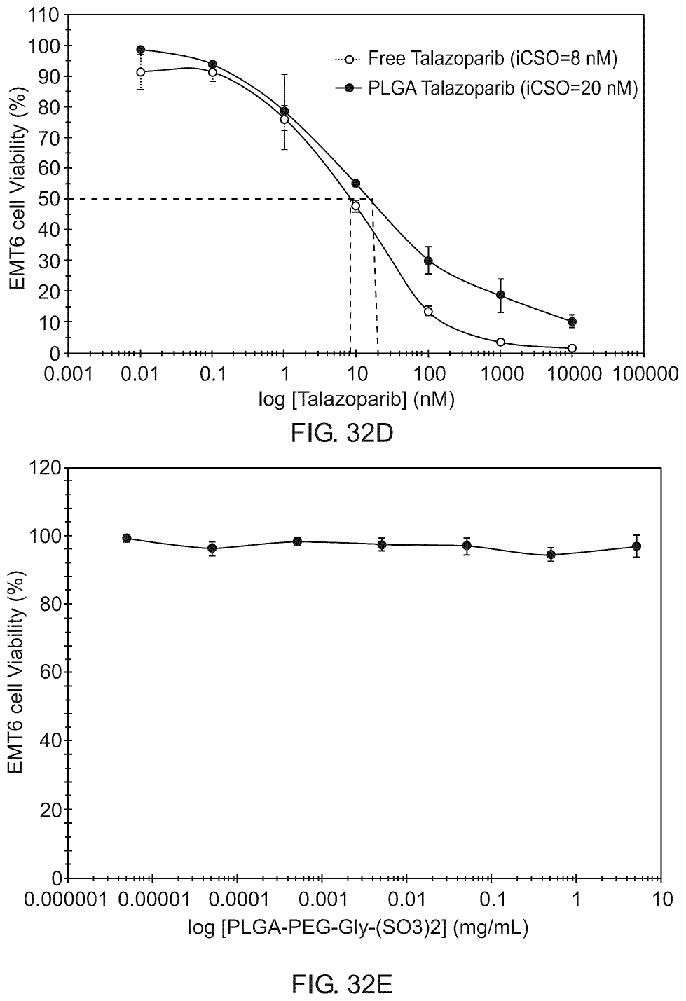

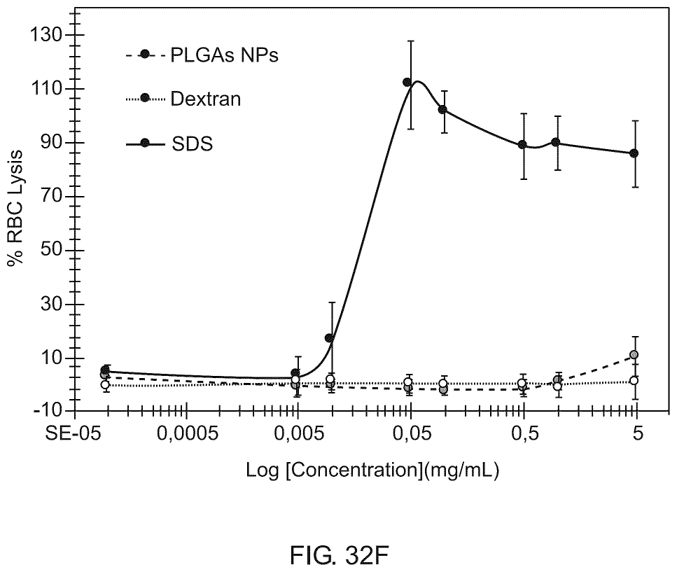

FIG. 32A is a graph showing the release profile of the exemplary compounds, talazoparib and SM56, from the single- and dual-loaded exemplary NPs (PLGA-PEG-Glycerol-(SO₃)₂), as indicated, in PBS.

FIGS. 32B-C are bar graphs showing polydispersity index (PDI; FIG. 32B) and hydrodynamic diameter by DLS (number; FIG. 32C) of the exemplary PLGA-PEG-Glycerol-(SO₃)₂ NPs after incubation in PBS for 48 hours. The graphs represent mean±SD, N=3.

FIGS. 32D-E are graphs showing EMT6 cell proliferation following incubation with free talazoparib and with the exemplary talazoparib-loaded PLGA-PEG NPs (FIG. 32D) or with blank (unloaded, carrier only) PLGA-PEG-Glycerol-(SO₃)₂ NPs (FIG. 32E).

FIG. 32F is a plot showing % red blood cells (RBC) lysis following a one hour-incubation with serial dilutions of the exemplary PLGA-PEG-Glycerol-(SO₃)₂ NPs, dextran (negative control), or sodium dodecyl sulfate (SDS, positive control), as indicated. The released percentage of haemoglobin was measured spectrophotometrically.

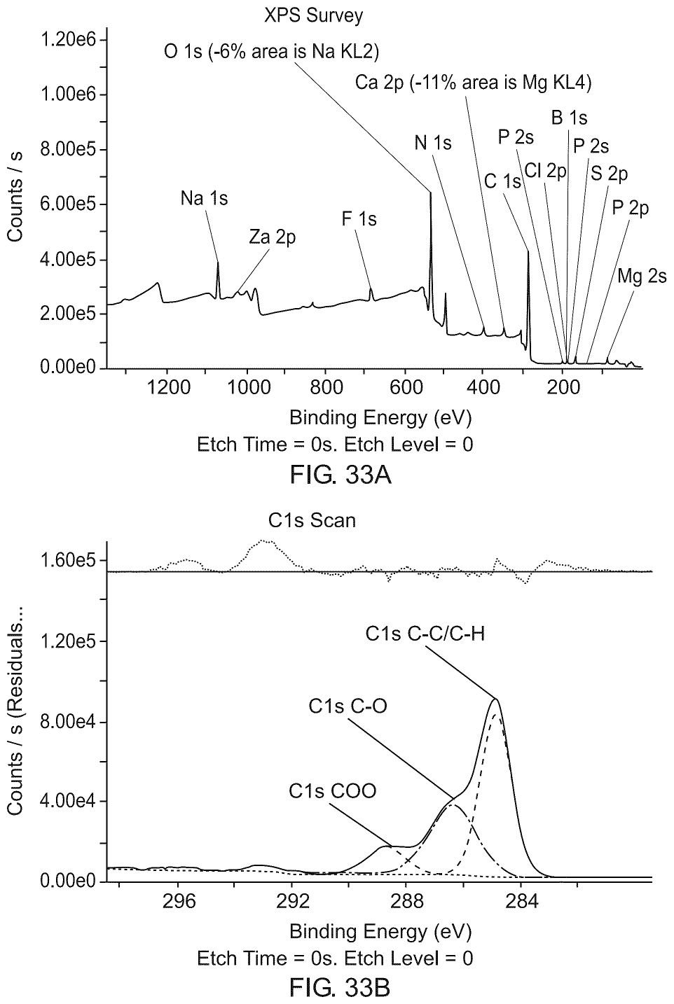

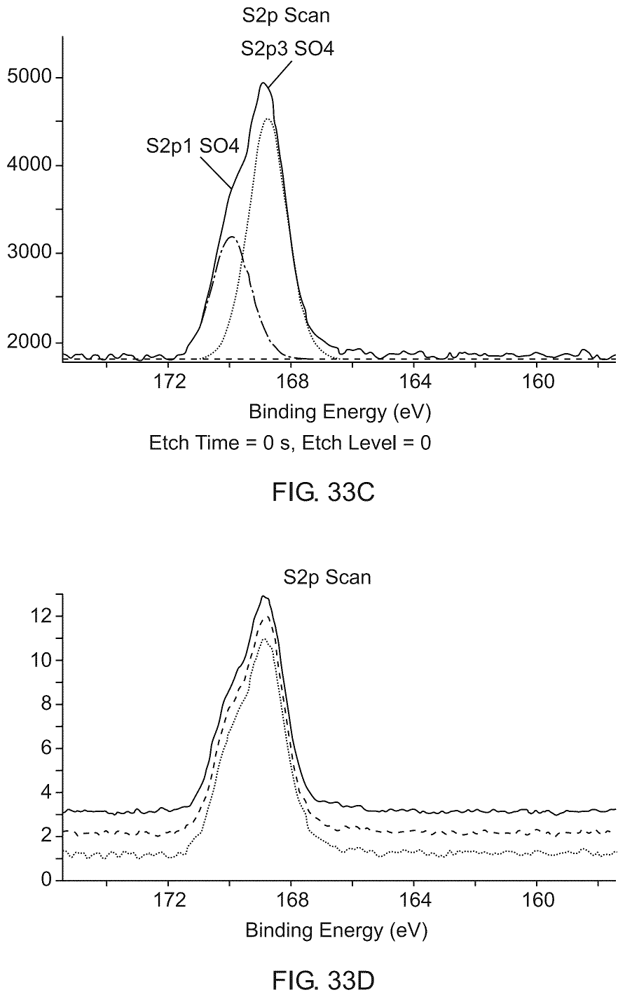

FIGS. 33A-D are XPS elemental analyses, indicating 20% presence of SO₄ groups on the surface of the exemplary PLGA-PEG-Glycerol-(SO₃)₂ NPs. FIG. 33A shows low resolution spectra of the XPS elemental analysis including all the species; FIG. 33B shows high resolution spectra of the reference peak—C1s (C—C bond); FIG. 33C shows high resolution spectra of the SO₄ peak; and FIG. 33D shows high resolution spectra of SO₄ peak and S [At %] relative to C at three different sputtering levels.

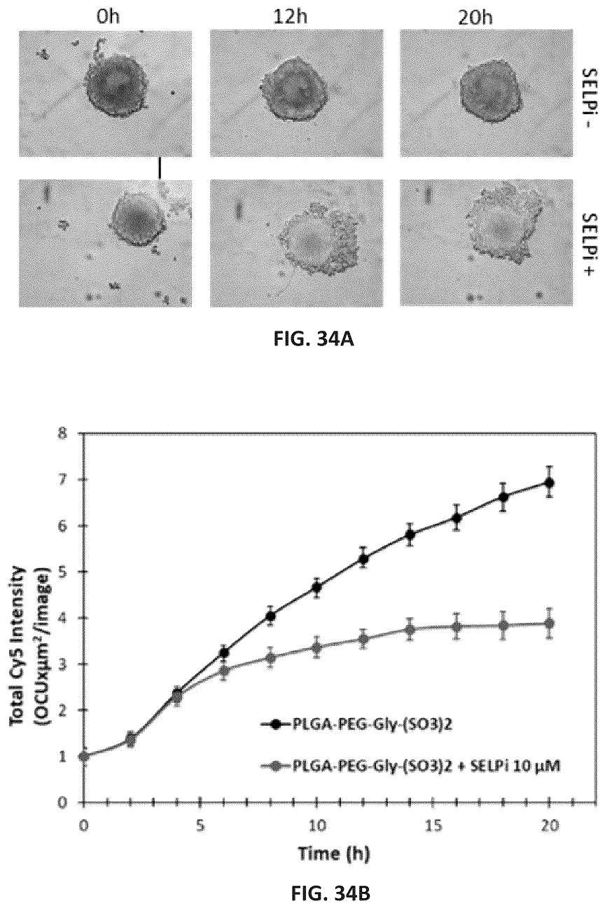

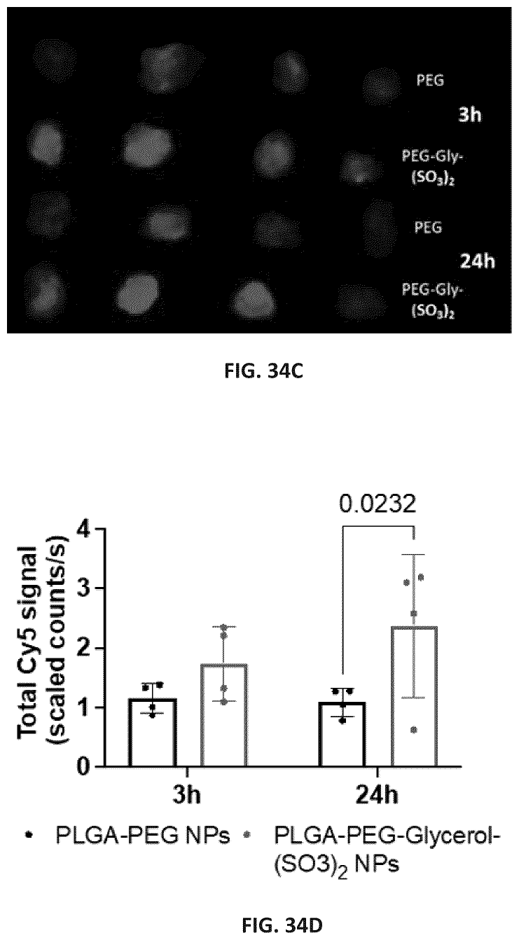

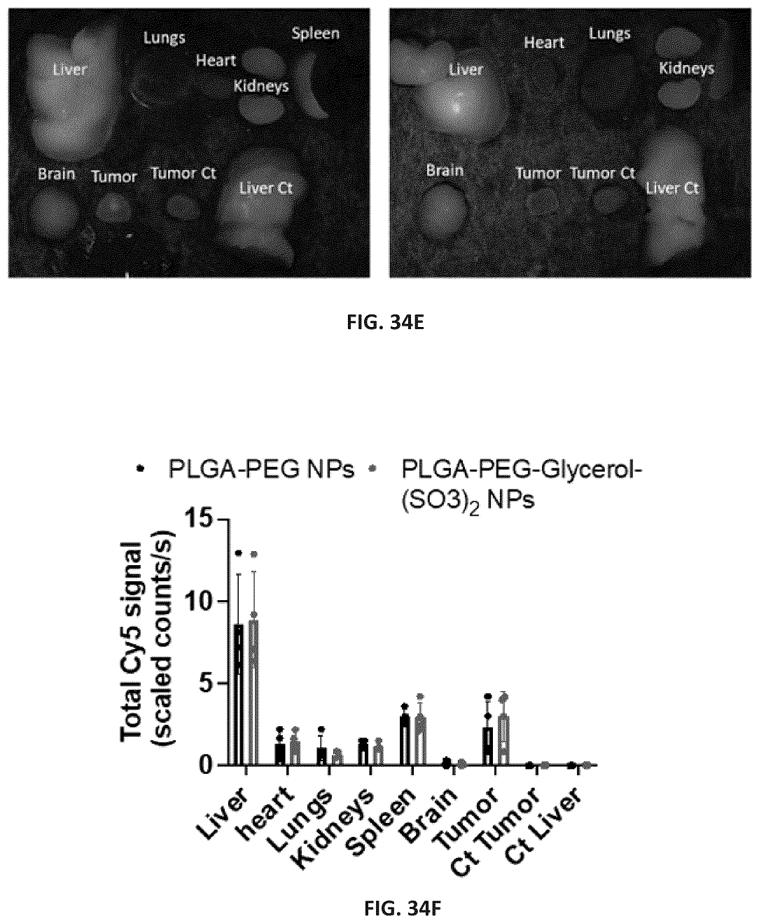

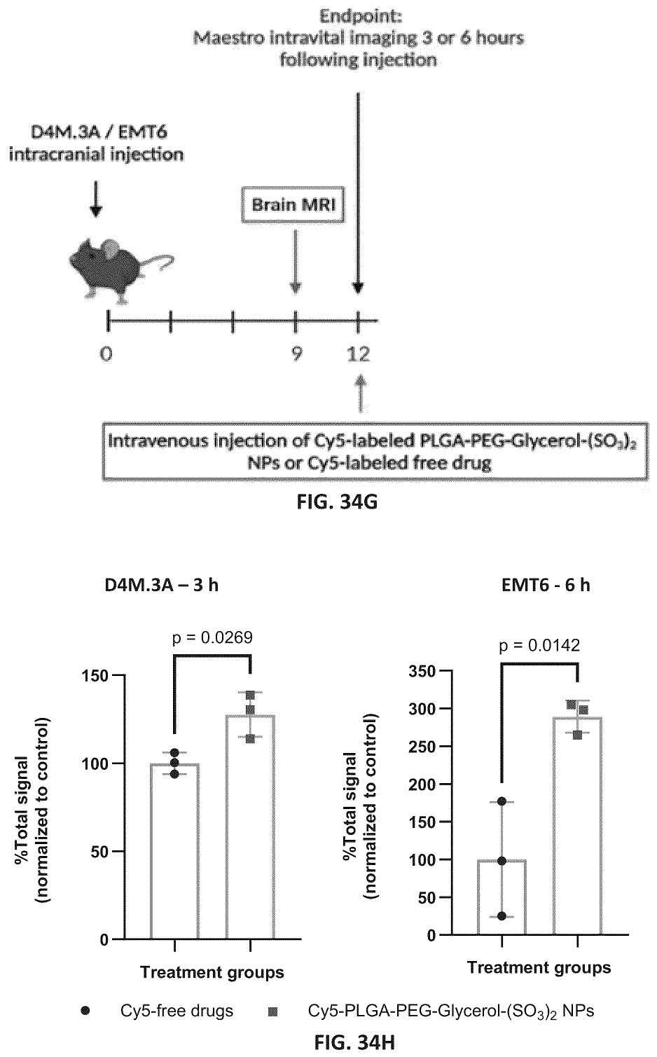

FIGS. 34A-B are representative images of Cy5-labeled PLGA-PEG-Glycerol-(SO₃)₂ NPs internalization into EMT6 spheroids with or without 10 μM SELPi, 1 hour prior to NPs addition (FIG. 34A) and the respective quantification of Cy5 intensity within the spheroids (FIG. 34B).

FIGS. 34C-D are representative images of the tumor accumulation of Cy5-labeled PLGA-PEG-Glycerol-(SO₃)₂ NPs or non-targeted NPs (PLGA-PEG NPs) 3 and 24 hours after the IV administration (FIG. 34C) and the respective quantification of the Cy5 signal from the EMT6 tumors (FIG. 34D).

FIGS. 34E-F are representative images of organs' Cy5 fluorescence signal 24 hours post-administration of Cy5-labeled PLGA-PEG-Glycerol-(SO₃)₂ and PLGA-PEG NPs (FIG. 34E) and a graph showing the respective quantification of the NPs distribution to essential organs 24 hours after the IV administration N=4 mice per group (FIG. 34F). Graph represents individual Cy5-intensity values from the tumor/organs and mean±SEM. Statistical significance was calculated using two-way ANOVA. The quantification was performed with the CRI Maestro, a non-invasive intravital fluorescence imaging system.

FIG. 34G is an experimental scheme, wherein Maestro intravital imaging of brains is performed 12 days after intracranial injection of EMT6 cells and 6 hours after the injection of Cy5-labeled exemplary PLGA-PEG-Glycerol-(SO₃)₂ NPs or the Cy5-labeled free exemplary drugs.

FIG. 34H is a bar graph showing tumor accumulation of a Cy5-labeled free drug (purple) or a Cy5-labeled PLGA-PEG-Glycerol-(SO₃)₂ drug-containing NPs (blue) in brain metastasis of D4M.A3 melanoma and EMT6 breast cancer. The brains were harvested and imaged 6 hours after the injection of the treatments. N=3 mice per group. The Graphs represent individual Cy5-intensity values from the tumor normalized to the free drug and mean±SEM. Statistical significance was calculated using student's t test.

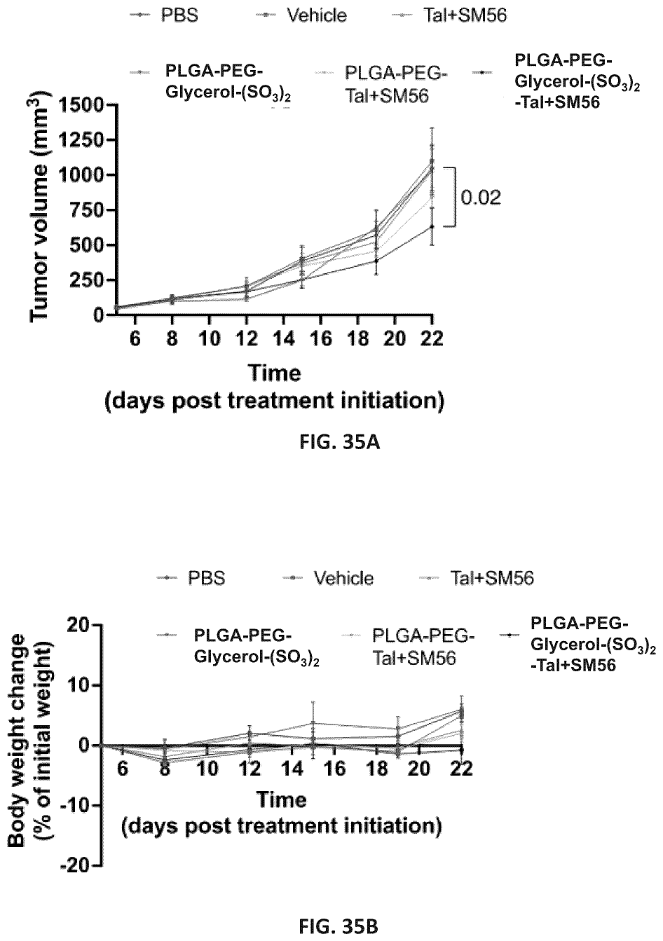

FIG. 35A is a graph showing the tumor volume growth over time. The p-values correspond to the tumor volume of the PLGA-PEG-Glycerol-(SO₃)₂ NPs group compared to PBS (p=0.016), vehicle (p=0.010) and free talazoparib and SM56 (p=0.020) at day 22. Data are shown as mean±SEM. Statistical significance was determined using two-sided repeated-measures ANOVA (p-value <0.05). N=6-9 mice per group.

FIG. 35B is a graph showing the body weight change over time, expressed as percent change from treatment initiation.

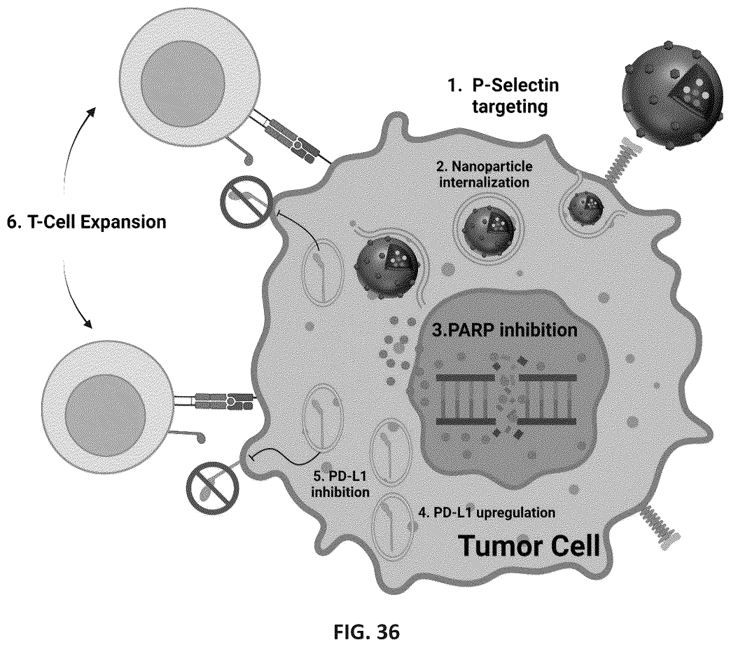

FIG. 36 is a schematic illustration of the suggested mechanism of action of the exemplary sulfated PLGA-PEG NPs, including 6 steps, as indicated and as further elaborated on Example 12.

### DESCRIPTION OF SPECIFIC EMBODIMENTS OF THE INVENTION

The present invention, in some embodiments thereof, relates to therapy and, more particularly, but not exclusively, to polymeric nanoparticles comprising therapeutically active agents, and to uses thereof in treating various conditions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As discussed hereinabove, P-selectin (SELP), a cell adhesion molecule responsible for leukocyte recruitment and platelet binding, is expressed constitutively in activated endothelial cells. Elevated SELP expression has been found at inflammation sites, and in patients suffering from various types of cancer. SELP was reported to promote metastasis and glioblastoma. The present inventors have previously described poly(lactic-co-glycolic acid) (PLGA)-polyethylene glycol (PEG)-based nanoparticles (NPs) designed to co-deliver a synergistic ratio of the BRAFi, dabrafenib (DBF) and the MEKi, trametinib (TRM), to the tumor site and enhance their therapeutic efficacy and safety.

While passive tumor accumulation is susceptible to variations in tumor vasculature, the present inventors have conceived modifying PLGA-based nanoparticles by introducing to their surface sulfate moieties to actively target the NPs to P-selectin (SELP)-expressing melanoma lesions. SELP offers dual targeting features as it is expressed on both activated endothelial cells at the tumor site and on the cancer (e.g., melanoma) cells.

By that, an increase was expected in the exposure of the tumor cells to the drugs, which hinders their ability to develop escape mechanisms. Indeed, exemplary novel drug-loaded NPs showed enhanced accumulation in SELP-expressing 3D spheroids.

These results were successfully translated into superior in-vivo efficacy for which the targeted NPs reduced tumor growth and prolonged the survival of mice compared to free drugs and non-targeted NPs.

Embodiments of the present invention relate to novel particles that bear a plurality of P-selectin targeting moieties and have therapeutically active agents associated therewith (e.g., encapsulated or entrapped in the particles), to compositions comprising same and to uses thereof in treating medical conditions associated with upregulated level of P-selecting.

According to an aspect of some embodiments of the present invention there is provided a composition comprising a plurality of particles, wherein in at least a portion of the particles, each particle comprises a polymeric matrix having associated therewith at least one therapeutically active agent usable in treating a medical condition associated with an overexpression of P-selectin in a subject in need thereof, wherein in at least a portion of the particles which comprise the polymeric matrix, the polymeric matrix has attached to a surface thereof a P-selectin (SELP) selective targeting moiety.

According to the present embodiments, P-selecting selective targeting moieties are collectively represented by Formula I:

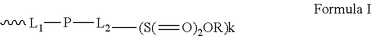

or a pharmaceutically acceptable salt thereof;
wherein:
R is hydrogen or alkyl;
the curved line represents an attachment point to the polymeric matrix;
P is an amphiphilic polymeric or oligomeric moiety;
L₁ and L₂ are each independently a linking moiety or absent; and
k is an integer ranging from 1 to 10, or from 1 to 6, or from 1 to 4, including any intermediate values and subranges therebetween.

The term “amphiphilic” as used herein and in the art describes a property of a material, or of a moiety derived therefrom, that combines both hydrophilicity (a physical property of a material or moiety which accounts for transient formation of bond(s) with water molecules, typically through hydrogen bonding) and hydrophobicity or lipophilicity (a physical property of a material or a portion of a material or moiety which does not form bond(s) with water molecules).

Amphiphilic materials typically comprise both hydrophilic and hydrophobic groups as defined herein, and are substantially soluble in both water and a water-immiscible solvent (oil).

Amphiphilic materials can be determined, for example, as having Log P of 0.8 to 1.2, or of about 1, when Log P is determined in octanol and water phases at ambient temperature (e.g. 25° C.).

Amphiphilic materials can alternatively, or in addition, be determined as featuring a lipophilicity/hydrophilicity balance (HLB), according to the Davies method, of 3 to 12, or 3 to 9.

According to some of the present embodiments, the amphiphilic polymers from which the polymeric moiety P is derived are biocompatible polymers.

Exemplary suitable amphiphilic polymers that can be used for deriving the polymeric moiety P include, but are not limited to, poly(alkylene glycols) as defined herein, including, for example, poly(ethylene glycol), poly(propylene glycol), poloxamers (triblock copolymers consisting of PEO and PPO blocks, poly(vinyl alcohol) (PVA), and more.

According to exemplary embodiments, P is or comprises a poly(alkylene glycol) moiety, as defined herein.

According to some of any of the embodiments described herein, an average molecular weight of the polymeric moiety ranges from about 100 to about 10,000, or from about 500 to about 5,000, or from about 1,000 to about 5,000, or from about 1,000 to about 3,000 grams/mol, including any intermediate values and subranges therebetween.

According to some of any of the embodiments described herein, the polymeric moiety P is or comprises a poly(ethylene glycol) having an average molecular weight that ranges from about 100 to about 10,000, or from about 500 to about 5,000, or from about 1,000 to about 5,000, or from about 1,000 to about 3,000 grams/mol, including any intermediate values and subranges therebetween.

According to some of any of the embodiments described herein, the L₁ and L₂ linking moieties, when present, are each independently selected from alkyl, aminoalkyl, hydroxyalkyl, heteroalicyclic, heteroaryl, thioalkyl, ether, thioether, —O—, —S—, amine, imine, —C(═O)—, —C(═S)—, amide, carbamate, carboxylate, thiocarboxylate, thiocarbamate, thioamide, sulfonate, sulfoxide, phosphonate, sulfonamide, urea, thiourea, hydrazine, hydrazide, and any combination thereof. The L₁ and L₂ linking moieties can be the same or different.

The L₁ linking moiety, if present, is used to attach the polymeric moiety P to the surface of the polymeric matrix, and can be selected in accordance with the surface groups of the polymeric matrix and/or the terminal groups of the polymeric moiety P, and a selected reaction used to couple the polymeric moiety P to the polymeric matrix.

For example, when P is a poly(alkylene glycol) and the polymeric matrix feature carboxylate (e.g., carboxylic acid) surface groups, conjugation of the polymeric moiety P to the polymeric matrix can be effected by addition-elimination reactions, to provide, for example, an ester bond (—C(═O)—O—) as the linking moiety. Optionally, and preferably, the one or more of the terminal group of the polymeric moiety P and the surface groups of the polymeric matrix is/are modified to generate groups the provide upon conjugation a linking moiety or bond that is less hydrolysable in vivo, and is therefore non-biodegradable, so as to assure that the targeting moiety remains attached to the polymeric matrix upon administration.

In exemplary embodiments, the linking moiety is an amine linking moiety, or an aminoalkyl linking moiety, that forms a relatively amide bond with carboxylate (e.g., carboxylic acid) surface groups of the polymeric matrix.

According to some of any of the embodiments described herein, L₁ is or comprises an amine (—NR′—) or an aminoalkyl.

The L₂ moiety, if present, is used to attach the sulfate or sulfonate moiety to the polymeric moiety, and can be a bond (e.g., —O—, —S—, —C(═O)—, —C(═S)—, a carbamate bond, a carboxylate bond, an amide bond, a thioamide bond, etc.) or a moiety.

According to some embodiments, the L₂ linking moiety, if present, is or comprises a hydrocarbon chain, as defined herein, optionally interrupted by one or more heteroatoms or substituent groups, as defined herein, and/or substituted by one or more substituents, as defined herein, which maintain the amphiphilic nature of the targeting moiety. For example, the hydrocarbon chain can be interrupted, substituted and/or comprise, one or more of an amine, aminoalkyl, hydroxyalkyl, thioalkyl, a heteroaryl, a heteroalicyclic, imine, ether, thioether, —O—, —S—, —C(═O)—, —C(═S)—, amide, carbamate, carboxylate, thiocarboxylate, thiocarbamate, thioamide, sulfonate, sulfoxide, phosphonate, sulfonamide, urea, thiourea, hydrazine, hydrazide, and any combination thereof, whereby each can be a linking group, as defined herein, if interrupting the hydrocarbon, or a terminal or end group, if substituting the hydrocarbon chain.

According to some embodiments, the hydrocarbon chain is a linear chain. According to some embodiments, the hydrocarbon chain is a saturated chain. According to some embodiments, the hydrocarbon chain is a linear saturated chain (e.g., an alkylene chain).

According to some embodiments, the hydrocarbon chain is of from 1 to 20, or from 1 to 10, or from 1 to 8, or from 1 to 6, carbon atoms in length, including any intermediate values and subranges therebetween.

According to some of any of the embodiments described herein, L₂ is or comprises an amine linking moiety or an aminoalkyl.

According to some of any of the embodiments described herein, each of L₁ and L₂ is or comprises an amine or an aminoalkyl, which can be the same or different.

According to some of any of the embodiments described herein, L₂ is a hydrocarbon chain as defined herein, interrupted by one or more amine linking groups.

According to some of any of the embodiments described herein, L₂ is a hydrocarbon chain as defined herein, interrupted by one or more —S— and/or —O— groups.

According to some of any of the embodiments described herein, L₂ is a hydrocarbon chain as defined herein, interrupted by one or more —S— groups.

According to some of any of the embodiments described herein, L₂ is a hydrocarbon chain as defined herein, interrupted by one or more carbamate and/or thiocarbamate groups.

According to some of any of the embodiments described herein, L₂ is a hydrocarbon chain as defined herein, interrupted by one or more amide and/or thioamide groups.

According to some of any of the embodiments described herein, L₂ is a hydrocarbon chain as defined herein, interrupted or substituted by one or more heteroaryl groups. In exemplary embodiments, the heteroaryl is formed by Click reaction.

According to some of any of the embodiments described herein, k is an integer of from 1 to 4, and can be 1, 2, 3 or 4. According to some of any of the embodiments described herein, k is 1, 2 or 3. According to some of any of the embodiments described herein, k is 1 or 2.

According to exemplary embodiments, k is 1, and the targeting moiety is a mono-sulfated (mono-sulfonate-containing) targeting moiety.

According to exemplary embodiments, k is 1 and the polymeric moiety P is a poly(alkylene glycol) as defined herein, for example, PEG.

According to exemplary embodiments, k is 1 and the targeting moiety can be represented by Formula II:

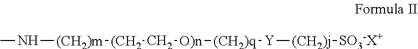

wherein n is an integer of at least 10, or at least 20; each of m, q and j is independently 0, 1, 2, 3 or 4; Y is —NH—, —O—, —S—, —C(═O)—, —C(═S)—, a carbamate bond, a carboxylate bond, an amide bond, a thioamide bond, etc.; and X⁺ is a monocation.

In exemplary embodiments, Y is —NH—.

In exemplary embodiments, m is 0 or 1.

In exemplary embodiments, q and j are each other than 0, and each independently is, for example, 1, 2 or 3.

In exemplary embodiments, Y is —NH—, m is 0, and q and j are each other than 0, and each independently is, for example, 1, 2 or 3.

According to some of any of the embodiments described herein, k is 2 or a higher integer, and L₂ is or comprises a branching unit.

The term “branching unit” as used herein throughout describes a multi-radical, preferably aliphatic or alicyclic, linking moiety. By “multi-radical” it is meant that the linking moiety has two or more attachment points such that it links between two or more atoms and/or groups or moieties.

That is, the branching unit is a chemical moiety that, when attached to a single position, group or atom of a substance, creates two or more functional groups that are linked to this single position, group or atom, and thus “branches” a single functionality into two or more functionalities.

In some embodiments, the branching unit is derived from a chemical moiety that has two, three or more functional groups, depending on the value of k.

In some embodiments, the branching unit is derived from glycerol, featuring at least two hydroxy groups that are available to form the two or more sulfonate moieties.

In exemplary embodiments, the branching unit is derived from glycerol, and has the following structure:

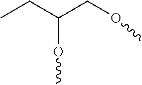

wherein the curved lines represent the attachment points to the SO₃R moieties, in case where k is 2, and the remaining position is connected to the remaining portion of L₂ or directly to the polymeric moiety P.

According to exemplary embodiments, k is 2, and the targeting moiety is a di-sulfated (mono-sulfonate-containing) targeting moiety. According to some of these embodiments, L₂ comprises a branching unit as described herein.

According to exemplary embodiments, k is 2 and the polymeric moiety P is a poly(alkylene glycol) as defined herein, for example, PEG. According to some of these embodiments, L₂ comprises a branching unit as described herein.

In exemplary embodiments, k is 2, the polymeric moiety P is a poly(alkylene glycol) as defined herein, for example, P, and L₂ comprises a branching unit derived from glycerol, as described herein, and the targeting moiety is represented by Formula III:

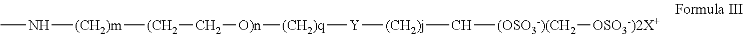

wherein n is an integer of at least 10, or at least 20; each of m, q and j is independently 0, 1, 2, 3 or 4; Y is —NH—, —O—, —S—, —C(═O)—, —C(═S)—, a carbamate bond, a carboxylate bond, an amide bond, a thioamide bond, a heteroaryl, a heteroalicyclic, etc. (as described herein); and X⁺ is a monocation.

In exemplary embodiments, Y is —NH—C(═O)—O— (a carbamate bond).

In exemplary embodiments, Y is —S—.

In exemplary embodiments, m is 0 or 1.

In exemplary embodiments, q and j are each other than 0, and each independently is, for example, 1, 2 or 3.

In exemplary embodiments, Y is —S— or a carbamate bond, m is 0, and q and j are each other than 0, and each independently is, for example, 1, 2 or 3.

According to some of any of the embodiments described herein, at least a portion, preferably a major portion, or each, of the particles are nanoparticles, having at least one dimension (e.g., diameter) within the nanoscale range (e.g., from 1 to 1,000 nanometer, or nm).

In exemplary embodiments, k is 4, and the branching unit has a dendritic structure in which two glycerol branching units as described herein extend from a third branching unit, as exemplified in FIG. 28C. Any other branching units that feature 4 functional groups to which a sulfonate or sulfate moieties can be attached are contemplated.

According to some embodiments, the nanoparticles have a diameter within the nanoscale range, of, for example, from 1 to 1,000, or from 1 to 800, or from 1 to 500, or from 1 to 300, or from 1 to 200, or from 100 to 200, or from 10 to 500, or from 10 to 300, or from 10 to 200, or from 10 to 100, or from 50 to 500, or from 50 to 500, or from 50 to 200, or from 50 to 150, nm, including any intermediate values and subranges therebetween.

The particles as described herein are also referred to herein as sulfonated or sulfated particles, or as particles featuring sulfonate groups.

The polymeric matrix that forms the particles can be made or any biocompatible, preferably FDA-approved, and further preferably biodegradable, polymeric material. Non-limiting examples include poly(lactic acid-co-glycolic acid (PLGA), poly(ethylene glycol) (PEG), Poly(hydroxyalkanoates) (PHA), Polydioxanone (PDO), Polyethylene Glycol-b-poly(lactic acid) (PRG-PLA, Chitosan, Poly(glycerol sebacate (PGS), Poly(trimethylene carbonate (PTMC), Poly(s-caprolactone-co-ethylene glycol) (PCL-PEG) and poly(amino acids).

In exemplary embodiments, the polymeric matrix is or comprises PLGA.

PLGA has been approved by the Food and Drug Administration (FDA) and European Medicine Agency (EMA), and is listed in the US Pharmacopoeia (USP) as a pharmaceutical excipient. It is available in a wide range of molecular weights (MW), which can be selected to provide varying degradation rates, thus allowing control of the degradation rate. It has scalable and reproducible production methods. PLDA is biocompatible and is able to encapsulate hydrophilic and/or hydrophobic APIs (drugs, peptides, oligonucleotides).

In exemplary embodiments, a PLGA having Mw that ranges from 1 to 1000, or from 10 to 500, KDa is used as the polymeric matrix, including any intermediate values and subranges therebetween. Exemplary PLGAs and Mw ranges are described in the Examples section that follows.

In some embodiments, the plurality of particles (e.g., nanoparticles) are made of a polymeric matrix that is or comprises PLGA. Exemplary nanoparticles comprise PLGA and PLA, and may optionally further comprise a PLGA-PEG copolymer (e.g., block copolymer).

In some embodiments, the plurality of particles (e.g., nanoparticles) comprise, in addition to particles having the targeting moiety attached thereto, a plurality of PLGA and/or PLA particles (e.g., nanoparticles) and/or a plurality of PLGA-PEG nanoparticles. Whenever the composition comprises particles that do not comprise a targeting moiety as described herein, the portion of the particles having the targeting moiety attached thereto relative to the total amount of particles in the composition is in a range of from 10% to 90%, or from 10% to 80%, or from 10%, to 70%, or from 10%, to 60%, or from 10% to 50%, or from 10% to 40%, or from 10% to 30%, or from 10% to 20%, or from 20% to 90%, or from 20% to 80%, or from 20%, to 70%, or from 20%, to 60%, or from 20% to 50%, or from 20% to 40%, or from 20% to 30%, or from 30% to 90%, or from 30%, to 80%, or from 30%, to 70%, or from 30% to 60%, or from 30% to 40%, or from 40% to 90%, or from 40% to 80%, or from 40% to 70%, or from 40%, to 60%, or from 40%, to 50%, or from 50% to 90%, or from 50% to 80%, or from 50% to 70%, or from 50% to 60%, including any intermediate values and subranges therebetween.

Particles (e.g., nanoparticles) encapsulating or entrapping therein the agent(s) as described herein can be prepared by any of the methods known in the art for preparing polymeric particles.

In some embodiments, there are provided processes of preparing nanoparticles entrapping one or more therapeutically active agents and having a targeting moiety attached to a surface thereof, as described herein.

An exemplary method involves an emulsion-solvent removal technique, such as, for example, double emulsion-solvent evaporation (w/o/w) technique, in which an organic solution comprising the polymeric substance is emulsified once or twice with an aqueous solution. The agent is added to the organic and/or aqueous solution, depending on its solubility. Emulsification can be performed by any technique known in the art, e.g. probe sonicator.

Another exemplary method involves a use of a microfluidic chip to which an organic solution containing the polymeric substance and the agent(s) and an aqueous solution are added.

The targeting moiety can be attached to the polymeric particle or to a polymer from which the nanoparticle is prepared, prior to its preparation.

An exemplary method of preparing the particles is provided in the Examples section that follows.

A composition as described herein in any of the respective embodiments is aimed at targeting over-expressed P-selectin, whereby the P-selectin is overexpressed as a result of a medical condition.

According to some of any of the embodiments described herein, the medical condition is a SELP-expressing cancer.

By “SELP-expressing cancer” it is meant a cancer in which tumor cells and/or cells in the tumor's microenvironment (e.g., endothelial cells) express or overexpress P-selecting. P-selectin expression or overexpression can be inherent to the tumor or can be induced by, for example, a co-therapy, for example, can be irradiation-induced.

According to some of any of the embodiments described herein, the medical condition is selected from melanoma, primary brain cancer (e.g., glioblastoma), brain metastases (originating from melanoma, lung cancer, breast cancer and colorectal cancer), colon cancer, pancreatic cancer, non-small cell lung cancer, ovarian carcinoma, head and neck squamous cell carcinoma, breast cancer (including BRCA-mutated breast cancer, HER2-positive breast cancer, HER2-negative breast cancer, triple negative breast cancer; TNBC), kidney cancer (e.g., renal cell carcinoma), pediatric glioma (e.g., pediatric low-grade glioma, DIPG, medulloblastoma, pilocytic astrocytoma followed by ganglioglioma, papillary craniopharyngioma), metastases thereof, and inflammation.

Additional cancers that may be SELP-expressing include, but are not limited to, any solid or non-solid cancer and/or tumor metastasis, including, but not limiting to, tumors of the gastrointestinal tract (e.g., colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic breast cancer, susceptibility to breast cancer, type 4 breast cancer, breast cancer-1, breast cancer-3, breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B-cell lymphoma, Diffuse large B-cell lymphoma (DLBCL), Burkitt lymphoma, cutaneous T-cell lymphoma, histiocytic lymphoma, lymphoblastic lymphoma, T-cell lymphoma, thymic lymphoma), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependymoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B-cell), hypernephroma, insulinoma, islet tumor, keratoma, leiomyoblastoma, leiomyosarcoma, leukemia (e.g., acute lymphatic leukemia, acute lymphoblastic leukemia, acute lymphoblastic pre-B cell leukemia, acute lymphoblastic T cell leukemia, acute megakaryoblastic leukemia, monocytic leukemia, acute myelogenous leukemia, acute myeloid leukemia, acute myeloid leukemia with eosinophilia, B-cell leukemia, basophilic leukemia, chronic myeloid leukemia, chronic B-cell leukemia, eosinophilic leukemia, Friend leukemia, granulocytic or myelocytic leukemia, hairy cell leukemia, lymphocytic leukemia, megakaryoblastic leukemia, monocytic leukemia, monocytic-macrophage leukemia, myeloblastic leukemia, myeloid leukemia, myelomonocytic leukemia, plasma cell leukemia, pre-B cell leukemia, promyelocytic leukemia, subacute leukemia, T-cell leukemia, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme, multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, Turcot syndrome with glioblastoma.

The therapeutically active agents associated with the polymeric matrix are selected as usable in the treating the medical condition. When the medical condition is a SELP-expressing cancer, the therapeutically active agent can be, or can comprise, an anti-cancer agent or a combination of two or more anti-cancer agents, or a combination of an anti-cancer agent and an additional agent that can act synergistically or additively with the anti-cancer agent.

When the medical condition is inflammation, or even when it is a SELP-expressing cancer, the therapeutically active agent can comprise one or more anti-inflammatory agent(s).

Anti-cancer drugs that can be used as therapeutically active agent include, but are not limited to, Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-Ia; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division). Any other anti-cancer drug is contemplated.

An additional list of anti-cancer (e.g., chemotherapeutic) drugs that can be used as therapeutically active agent includes, but is not limited to, abarelix, aldesleukin, aldesleukin, alemtuzumab, alitretinoin, allopurinol, altretamine, amifostine, anastrozole, arsenic trioxide, asparaginase, azacitidine, bevacizumab, bexarotene, bleomycin, bortezomib, busulfan, calusterone, capecitabine, carboplatin, carmustine, celecoxib, cetuximab, cisplatin, cladribine, clofarabine, cyclophosphamide, cytarabine, dacarbazine, dactinomycin, actinomycin D, Darbepoetin alfa, Darbepoetin alfa, daunorubicin liposomal, daunorubicin, decitabine, Denileukin diftitox, dexrazoxane, dexrazoxane, docetaxel, doxorubicin, dromostanolone propionate, Elliott's B Solution, epirubicin, Epoetin alfa, erlotinib, estramustine, etoposide, exemestane, Filgrastim, floxuridine, fludarabine, fluorouracil 5-FU, fulvestrant, gefitinib, gemcitabine, gemtuzumab ozogamicin, goserelin acetate, histrelin acetate, hydroxyurea, Ibritumomab Tiuxetan, idarubicin, ifosfamide, imatinib mesylate, interferon alfa 2a, Interferon alfa-2b, irinotecan, lenalidomide, letrozole, leucovorin, Leuprolide Acetate, levamisole, lomustine, CCNU, meclorethamine, nitrogen mustard, megestrol acetate, melphalan, L-PAM, mercaptopurine 6-MP, mesna, methotrexate, mitomycin C, mitotane, mitoxantrone, nandrolone phenpropionate, nelarabine, Nofetumomab, Oprelvekin, Oprelvekin, oxaliplatin, paclitaxel, palifermin, pamidronate, pegademase, pegaspargase, Pegfilgrastim, pemetrexed disodium, pentostatin, pipobroman, plicamycin mithramycin, porfimer sodium, procarbazine, quinacrine, Rasburicase, Rituximab, sargramostim, sorafenib, streptozocin, sunitinib maleate, tamoxifen, temozolomide, teniposide VM-26, testolactone, thioguanine 6-TG, thiotepa, thiotepa, topotecan, toremifene, Tositumomab, Trastuzumab, tretinoin ATRA, Uracil Mustard, valrubicin, vinblastine, vinorelbine, zoledronate and zoledronic acid. Any other chemotherapeutic drug is contemplated.

Anti-inflammatory drugs that can be used as therapeutically active agents, for example, in combination with an anti-cancer drug, include but are not limited to, Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium. Any other anti-inflammatory drug is contemplated.

An additional list of targeted therapies that can be used as therapeutically active agent includes, but is not limited to, Bevacizumab, Cobimetinib, Regorafenib, Trametinib, Binimetinib Olaparib (or an equivalent PARP inhibitor), Pembrolizumab (or an equivalent PD1 inhibitor), Sorafenib, Cetuximab, Ramucirumab, Ipilimumab, Erlotinib, Pazopanib, Ripretinib, Crizotinib, Everolimus (or an equivalent MTOR inhibitor), Cabozantinib, Selinexor, Imatinib (or an equivalent ABL inhibitor), Selpercatinib, Ribociclib (or an equivalent CDK4/6 inhibitor), Tivozanib, Alpelisib, Sunitinib, Lenvatinib, Axitinib, Selumetinib.

According to some of any of the embodiments described herein, the at least one therapeutically active agent is selected from a MEK inhibitor (e.g., pimasertib, binimetinib, cobimetinib, refametinib, selumetinib, trametinib, mirdametinib (PD325901), PD318088, PD334581, PD98059, PD184352 (CI-1040), AZD6244 (ARRY-142886), RDEA119, MEK162 (ARRY-438162)); a BRAF inhibitor (e.g., encorafenib (LGX818), dabrafenib, vemurafenib, sorafenib, GDC-0879, N-[3-(5-chloro-1H-pyrrolo[2,3-b]pyridin-3-ylcarbonyl)-2,4-difluorophenyl]propane-1-sulfonamide (PLX4720), (3R)—N-(3-[[5-(2-cyclopropylpyrimidin-5-yl)-1H-pyrrolo[2,3-b]pyridin-3-yl]carbonyl]-2,4-difluorophenyl)-3-fluoropyrrolidine-1-sulfonamide (PLX8394)); an EGFR inhibitor (e.g., cetuximab, panitumumab, osimertinib (merelectinib), erlotinib, gefitinib, necitumumab, neratinib, lapatinib, vandetanib, brigatinib); a poly ADP ribose polymerase (PARP) inhibitor (e.g., talazoparib, olaparib, rucaparib, niraparib, veliparib, pamiparib, iniparib, CEP9722, E7016); an inhibitor of HER2 and/or HER3 (e.g., lapatinib, trastuzumab, AC-480, erlotinib, gefitinib, afatinib, neratinib, CDX-3379, U-31402, HMBD-001, MCLA-128, KBP-5209, poziotinib, varlitinib. FCN-411, elgemtumab, sirotinib); a SHP2 inhibitor (e.g., 6-(4-amino-4-methylpiperidin-1-yl)-3-(2,3-dichlorophenyl)pyrazin-2-amine (SHP099), [3-[(3S,4S)-4-amino-3-methyl-2-oxa-8-azaspiro[4.5]decan-8-yl]-6-(2,3-dichlorophenyl)-5-methylpyrazin-2-yl]methanol (RMC-4550) RMC-4630, TNO155); an Axl inhibitor (e.g., R428, BGB324, crizotinib, bosutinib, cabozantinib, sunitinib, foretinib, merestinib, glesatinib), a PI3K inhibitor (e.g., buparlisib (BKM120), alpelisib (BYL719), samotolisib (LY3023414), 8-[(1R)-1-[(3,5-difluorophenyl)amino]ethyl]-N,N-dimethyl-2-(morpholin-4-yl)-4-oxo-4H-chromene-6-carboxamide (AZD8186), tenalisib (RP6530), voxtalisib hydrochloride (SAR-245409), gedatolisib (PF-05212384), panulisib (P-7170), taselisib (GDC-0032), trans-2-amino-8-[4-(2-hydroxyethoxy)cyclohexyl]-6-(6-methoxypyridin-3-yl)-4-methylpyrido[2,3-d]pyrimidin-7(8H)-one (PF-04691502), duvelisib (ABBV-954), N2-[4-oxo-4-[4-(4-oxo-8-phenyl-4H-1-benzopyran-2-yl)morpholin-4-ium-4-ylmethoxy]butyryl]-L-arginyl-glycyl-L-aspartyl-L-serine acetate (SF-1126), pictilisib (GDC-0941), 2-methyl-1-[2-methyl-3-(trifluoromethyl)benzyl]-6-(morpholin-4-yl)-1H-benzimidazole-4-carboxylic acid (GSK2636771), idelalisib (GS-1101), umbralisib tosylate (TGR-1202), pictilisib (GDC-0941), copanlisib hydrochloride (BAY 84-1236), dactolisib (BEZ-235), 1-(4-[5-[5-amino-6-(5-tert-butyl-1,3,4-oxadiazol-2-yl)pyrazin-2-yl]-1-ethyl-1H-1,2,4-triazol-3-yl]piperidin-1-yl)-3-hydroxypropan-1-one (AZD-8835), 5-[6,6-dimethyl-4-(morpholin-4-yl)-8,9-dihydro-6H-[1,4]oxazino[4,3-e]purin-2-yl]pyrimidin-2-amine (GDC-0084) everolimus, rapamycin, perifosine, sirolimus, temsirolimus); a SOS1 inhibitor (e.g., BI-3406), a signal transduction pathway inhibitor (e.g., Ras-Raf-MEK-ERK pathway inhibitors, PI3K-Akt-mTOR-S6K pathway inhibitors (PI3K inhibitors)), a CTLA-4 inhibitor (e.g., ipilimumab, tremelimumab), an apoptosis pathway modulator (e.g., camptothecin), a cytotoxic chemotherapeutic agent (e.g., irinotecan), an anti-angiogenesis agent (e.g., paclitaxel, axitinib, bevacizumab, cabozantinib, everolimus, lenalidomide, lenvatinib mesylate, pazopanib, ramucirumab, regorafenib, sorafenib, sunitinib, thalidomide, vandetanib, ziv-aflibercept), a PD-1 and/or PD-L1 inhibitor (e.g., atezolizumab, avelumab, durvalumab, KN035, cosibelimab (CK-301), AUNP12, CA-170, BSM-986189, cemiplimab, dostarlimab, nivolumab, pembrolizumab (MK-3475), vopratelimab (JTX-4014), spartalizumab (PDR001), camrelizumab (SHR1210), sintilimab (IBI308), tislelizumab (BGB-A317), toripalimab (JS 001), INCMGA00012 (MGA012), AMP-224, AMP-514), an immune-check-point regulator (e.g., modulators of PD-1, PD-L1, B7H2, B7H4, CTLA-4, CD80, CD86, LAG-3, TIM-3, KIR, IDO, CD19, OX40, 4-1BB (CD137), CD27, CD70, CD40, GITR, CD28 and ICOS (CD278), CCL2, CCR2), a topoisomerase (e.g., TOP1) inhibitor (e.g., camptothecin derivatives such as topotecan and irinotecan, anthracyclines such as doxorubicin and daunorubicin, epipodophyllotoxins such as etoposide and teniposide, flavopiridol, ixabepilone, belomustine, lurtotecan), an ataxia telangiectasia and Rad3 related (ATR) kinase inhibitor (e.g., berzosertib (VX-970, M6620), VE-821, AZD6738, KU-60019, BAY-59-8862), a VEGF receptor tyrosine kinase inhibitor (e.g., tivozanib), a CDK4 and/or CDK6 inhibitor (e.g., ribociclib), a receptor tyrosine kinase RET (rearranged during transfection) inhibitor (e.g., selpercatinib), a KIT proto-oncogene receptor tyrosine kinase (KIT) inhibitor (e.g., ripretinib), a mTOR inhibitor (e.g., sirolimus, temsirolimus, ridaforolimus, AZD2014), a selective inhibitor of nuclear export (SINE) (e.g., selinexor), an ABL inhibitor (e.g., imatinib, nilotinib, dasatinib, busatinib, ponatinib), and an agent capable of interfering with an interaction between PD-1 and PD-L1 (e.g., any of the compounds disclosed in WO 2022/175955).

According to some of any of the embodiments described herein, the composition comprises two or more of the therapeutically active agents (e.g., any combination of two or more of the therapeutically active agent described herein).

According to some of any of the embodiments described herein, in at least a portion of the particles which comprise a polymeric matrix having a therapeutically active agent associated therewith, each particle comprises the two or more therapeutically active agents. Without being bound by any particular theory, it is expected that such a co-encapsulation provides for enhanced therapeutic effect since the two therapeutically active agents are released simultaneously upon the SELP-targeting to a respective SELP-expressing cell. Such embodiments are advantageous particularly in cases where the two therapeutically active agents act synergistically or additively, as described herein, as it assures simultaneous accumulation of the drugs in the cells.

According to some of any of the embodiments described herein, in at least a portion of the particles which comprise a polymeric matrix having a therapeutically active agent associated therewith, each particle comprises one therapeutically active agent and in at least another portion of these particles, each particle comprises another, different, therapeutically active agent. These embodiments allow for a targeted co-administration of a drug combination in the same composition, which is otherwise is not enabled (e.g., due to incompatibility of the drugs with one another).

Any combination of two therapeutically active agents is contemplated. A ratio between the active agents or between different particles that bear different active agents can be determined based on the cumulative therapeutic effect of the selected agents.

In some of the embodiments where the composition comprises two (or more) therapeutically active agents, at least two therapeutically active agents act in synergy or at least exhibit an additive therapeutic effect.

In some of these embodiments, the two therapeutically active agents act in synergy in treating the medical condition as described herein in any of the respective embodiments, e.g., a medical condition associated with overexpression of P-selectin.

By “act in synergy” it is meant that when the two (or more) agents are contacted together with, for example, with cells expressing P-selectin, the therapeutic effect is higher than the additive effect when each agent acts alone. In some embodiments, the therapeutic activity is associated with inhibiting growth, proliferation, differentiation, migration and/or angiogenesis of the cells (over)expressing P-selectin.

By “act in synergy” it is also meant that a therapeutic effect of a plurality of particles bearing the two agents (either in the same particle or in different portions of the particles) is higher than the additive effect provided by particles one agent when used alone and particles bearing the other agent when used alone.

Synergy can be determined by methods known in the art. In some embodiments, synergy is determined by means of an isobologram, as widely described in the art.

When two or more therapeutically active agents act in synergy, at least one of the agents can be used, or included in the composition, in a sub-therapeutically effective amount (which is enabled by the synergistic effect).

In some of any of the embodiments described herein, the medical condition is a cancer associated with BRAF mutation. In some of these embodiments, the BRAF mutation is BRAF V600E or V600D.

A high frequency of a BRAF mutation is found, for example, in melanoma patients. Yet, a BRAF mutation is also found in substantial frequencies in other types of cancers.

Non-limiting examples of cancers which have been identified to date as associated with a BRAF mutation as described herein include glial cancers such as Pilocytic astrocytoma; Diffuse astrocytoma; Anaplastic astrocytoma; Oligodendroglioma; Anaplastic oligodendroglioma; Oligoastrocytoma; Anaplastic oligoastrocytoma; Primary glioblastoma; Secondary glioblastoma; Giant cell glioblastoma; Gliosarcoma; Gliomatosis cerebri; Ependymoma; Anaplastic ependymoma; Pleomorphic xanthoastrocytoma; Pleomorphic xanthoastrocytoma with anaplasia; Medulloblastoma; Ganglioglioma; Meningioma; and Pituitary adenoma, and other cancer types.

In some embodiments, the cancer is a CNS-metastasizing cancer.

Exemplary cancers include brain cancer (e.g., glioblastoma), non-small cell lung cancer (NSCLC), melanoma, and colon cancer.

In some embodiments, the cancer is glioblastoma.

In some embodiments, the cancer is melanoma.

In exemplary embodiments, the at least one therapeutically active agent is or comprises an agent that downregulates an activity of MEK and/or BRAF. According to some of these embodiments, the medical condition is a cancer associated with BRAF mutation, as described herein.

Exemplary agents that downregulate an activity of MEK and/or BRAF include, but are not limited to, pimasertib, binimetinib, cobimetinib, refametinib, selumetinib, trametinib, mirdametinib (PD325901), PD318088, PD334581, PD98059, PD184352 (CI-1040), AZD6244 (ARRY-142886), RDEA119, MEK162 (ARRY-438162), encorafenib (LGX818), dabrafenib, vemurafenib, sorafenib, GDC-0879, N-[3-(5-chloro-1H-pyrrolo[2,3-b]pyridin-3-ylcardonyl)-2,4-difluorophenyl]propane-1-sulfonamide (PLX4720), (3R)—N-(3-[[5-(2-cyclopropylpyrimidin-5-yl)-1H-pyrrolo[2,3-b]pyridin-3-yl]carbonyl]-2,4-difluorophenyl)-3-fluoropyrrolidine-1-sulfonamide (PLX8394), and structural analogs thereof.

By “structural analog” of a compound or agent it is meant a derivative of the compound (agent) which has a skeleton structure substantially the same as the compound or agent, yet one or more groups, preferably substituents of one or more positions of the skeleton structure, is/are replaced by another substituent(s), while retaining the biological activity of the compound. Structural analogs can be characterized as exhibiting a biological activity which is substantially the same as that of the original compound or agent. By “substantially the same” it is meant, for example, exhibiting an IC₅₀ under the same experimental condition which is ±20%, or ±10% or ±5% the IC₅₀ of the original compound or agent.

According to some of any of the embodiments described herein, in at least a portion of the particles, each particle comprises at least one agent that downregulates an activity of MEK and at least one agent that downregulates an activity of BRAF, that is, each particle comprises two therapeutically active agents.

According to some of any of the embodiments described herein, one portion of the particles comprises an agent that downregulates an activity of MEK as the therapeutically active agent and another portion of the particles comprises an agent that downregulates an activity of BRAF as the therapeutically active agent.

According to some of any of the embodiments described herein, the agent that downregulates an activity of MEK and the agent that downregulates an activity of BRAF act in synergy, as described herein.

In exemplary embodiments, the agent that downregulates the activity of MEK is trametinib.

In exemplary embodiments, the agent that downregulates the activity of BRAF is dabrafenib.

In exemplary embodiments, the agent that downregulates the activity of BRAF is dabrafenib and the agent that downregulates the activity of MEK is trametinib.

According to some of any of the embodiments described herein, at least one, or both, of the agent that downregulates an activity of MEK and the agent that downregulates an activity of BRAF is used in a sub-therapeutically effective amount. In exemplary embodiments, this agent is dabrafenib.

According to some of any of the embodiments described herein, the at least one therapeutically active agent is or comprises an immune checkpoint regulator, preferably an immune checkpoint inhibitor.

According to some of these embodiments, the medical condition is amenable to treatment by inhibiting PD-1, PD-L1 and/or the PD-1/PD-L1 interaction.

According to some of these embodiments, the medical condition is characterized by overexpression of PD-1 (e.g. lung cancer, melanoma, breast cancer, renal cancer, bladder cancer).

According to some of any of the embodiments described herein, the cancer is lung cancer. Examples of lung cancers which may be treated in the context of these embodiments of the invention include, without limitation, large (non-small) cell lung cancer and small cell lung cancer.

According to some of any of the embodiments described herein, the cancer is melanoma. Examples of melanoma treatable in the context of these embodiments include, without limitation, superficial spreading melanoma, lentigo melanoma, acral lentigous melanoma and nodular melanoma.

According to some of any of the embodiments described herein, the cancer is breast cancer. Examples of melanoma treatable in the context of these embodiments include, without limitation, Ductal carcinoma in situ (DCIS), invasive breast cancer (ILC or IDC), Triple-negative breast cancer, inflammatory breast cancer, Paget disease of the breast, Angiosarcoma and Phyllodes tumor.

According to some of any of the embodiments described herein, the cancer is colorectal cancer. Examples of colorectal cancer treatable in the context of these embodiments include, without limitation, Adenocarcinoma, Gastrointestinal carcinoid tumors, Primary colorectal lymphomas, Gastrointestinal stromal tumors, Leiomyosarcomas, Squamous cell carcinomas, Familial adenomatous polyposis (FAP), Turcot Syndrome, Peutz-Jeghers Syndrome (PJS), Familial Colorectal Cancer (FCC), and Juvenile Polyposis Coli.

According to some of any of the embodiments described herein, the cancer is bladder cancer. Examples of bladder cancers treatable in the context of these embodiments include, without limitation, transitional cell (urothelial) carcinoma (TCC), including papillary carcinoma and flat carcinomas, non-invasive bladder cancer, invasive bladder cancer, recurrent bladder cancer, metastatic bladder cancer, Squamous cell carcinoma, adenocarcinoma of the bladder, small-cell carcinoma and sarcoma.

As used herein the term “immune-check point regulator” refers to a molecule that modulates the activity of one or more immune-check point proteins in an agonistic or antagonistic manner resulting in activation of an immune cell.

As used herein the term “immune-check point inhibitor” refers to a molecule that modulates the activity of one or more immune-check point proteins in an antagonistic manner.

As used herein the term “immune-check point protein” refers to a protein that regulates an immune cell activation or function. Immune check-point proteins can be either co-stimulatory proteins (i.e. transmitting a stimulatory signal resulting in activation of an immune cell) or inhibitory proteins (i.e. transmitting an inhibitory signal resulting in suppressing activity of an immune cell). According to specific embodiment, the immune check point protein regulates activation or function of a T cell. Numerous checkpoint proteins are known in the art and include, but not limited to, PD1, PDL-1, B7H2, B7H4, CTLA-4, CD80, CD86, LAG-3, TIM-3, KIR, IDO, CD19, OX40, 4-1BB (CD137), CD27, CD70, CD40, GITR, CD28 and ICOS (CD278).

According to specific embodiments, the immune-check-point regulator is selected from anti-CTLA4, anti-PD-1, and CD40 agonist.

According to specific embodiments, the immune-check point regulator is selected from anti-CTLA4, anti-PD-1, anti-PDL-1, CD40 agonist, 4-1BB agonist, GITR agonist and OX40 agonist.

CTLA4 is a member of the immunoglobulin superfamily, which is expressed on the surface of Helper T cells and transmits an inhibitory signal to T cells upon ligand binding. As used herein, the term “anti-CTLA4” refers to an antagonistic molecule that binds CTLA4 (CD152) and suppresses its suppressive activity. Thus, an anti-CTLA4 prevents the transmission of the inhibitory signal and thereby acts as a co-stimulatory molecule. According to a specific embodiment, the anti-CTLA4 molecule is an antibody.

PD-1 (Programmed Death 1) is a member of the extended CD28/CTLA-4 family of T cell regulators which is expressed on the surface of activated T cells, B cells, and macrophages and transmits an inhibitory signal upon ligand binding. As used herein, the term “anti-PD1” refers to an antagonistic molecule that binds PD-1 and suppresses its suppressive activity. Thus, an anti-PD-1 prevents the transmission of the inhibitory signal and thereby acts as a co-stimulatory molecule.

PDL-1 is a ligand of PD-1. Binding of PDL-1 to its receptor PD-1 transmits an inhibitory signal to the cell expressing the PD-1. As used herein, the term “anti-PDL-1” refers to an antagonistic molecule that inhibits PD-1 signaling by binding to or inhibiting PDL-1 from binding and/or activating PD-1. Thus, an anti-PD-1 prevents the transmission of the inhibitory signal and thereby acts as a co-stimulatory molecule. According to some embodiments, the anti-PDL-1 is an anti-PDL1 antibody. Numerous anti-PDL-1 antibodies are known in the art (see, e.g., Brahmer, et al. NEJM 2012).

CD40 (CD154) is a co-stimulatory receptor found on antigen presenting cells and transmits an activation signal upon ligand binding. As used herein, the term “CD40 agonist” refers to an agonistic molecule that binds CD40 (CD154) and thereby induces activation of the antigen presenting cell.

OX40 belongs to the TNF receptor super family and leads to expansion of CD4+ and CD8+ T cells. As used herein, the term “OX40 agonist” refers to an agonistic molecule that binds and activates OX40.

GITR (glucocorticoid-induced tumor necrosis factor receptor) is a surface receptor molecule that has been shown to be involved in inhibiting the suppressive activity of T-regulatory cells and extending the survival of T-effector cells. As used herein, the term “GITR agonist” refers to an agonistic molecule that binds and activates GITR. According to a specific embodiment, the GITR agonist is an antibody.

In some embodiments, the immune checkpoint inhibitors may be an anti-PD-1, an anti-PD-L1 antibody, and/or an anti PD-1/PD-L1 interaction inhibitor. In some embodiments, the anti-PD-L1 antibody may be B7-H1 antibody, BMS 936559 antibody, MPDL3280A (atezolizumab) antibody, MEDI-4736 antibody, MSB0010718C antibody or combinations thereof. According to another embodiment, the anti-PD-1 antibody may be nivolumab antibody, pembrolizumab antibody, pidilizumab antibody or combinations thereof.

Examples of PD1 inhibitors include, without limitation, pembrolizumab, nivolumab, cemiplimab, spartalizumab, sintilimab, tislelizumab, toripalimab, dostarlimab, JTX-4014, INCMGA00012, AMP-224, and AMP-514.

Examples of PDL1 inhibitors, include, without limitation, atezolizumab, durvalumab, avelumab, KN035, CK-301, AUNP12, CA-170 and BMS-986189.

Any other inhibitors of PD1 and/or PD-L1 (PDL1) are also contemplated.

According to some of any of the embodiments described herein, the at least one therapeutically active agent is a PD1 and/or PDL1 inhibitor which is an agent that interferes with an activity or expression of PD1 and/or PDL1, and/or an agent that interferes with an interaction between PD1 and PDL1, which is also referred to herein as PD1 inhibitor or as PDL1 inhibitor.

Exemplary such agents are described in Acúrcio et al. Journal for ImmunoTherapy of Cancer, 10(7), e004695 (2022) and in WO 2022/175955, and include, without limitation, the following compound:

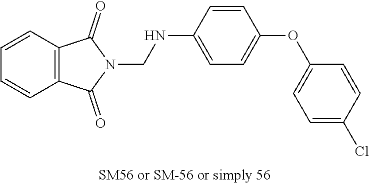 [SMILES: O=C1c2ccccc2C(=O)N1CNc1ccc(Oc2ccc(Cl)cc2)cc1]

which is mostly usable in assays conducted in murine cells, and preferably, one more of the following compounds:

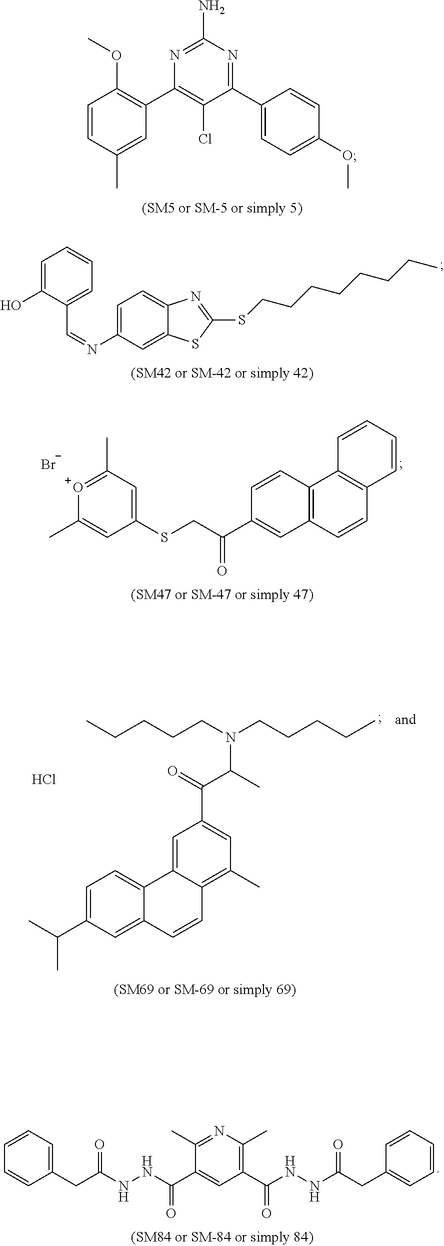

According to some of any of the embodiments described herein, the composition comprises two or more therapeutically active agents, as described herein in any of the respective embodiments, wherein one is a PD1 and/or PDL1 inhibitor, as described herein in any of the respective embodiments and any combination thereof, including a PD1 and/or PDL1 inhibitor which is an agent that interferes with an activity or expression of PD1 and/or PDL1, and/or an agent that interferes with an interaction between PD1 and PDL1 as described herein.

According to some of these embodiments, the two or more therapeutically active agents further comprise a PARP inhibitor. Exemplary PARP inhibitors include, without limitation, talazoparib, olaparib, rucaparib, niraparib, and veliparib.

According to some of any of the embodiments described herein, the medical condition is amenable to treatment by inhibiting PARP activity. Exemplary such medical conditions include cancers associated with BRCA mutation, including BRCA1 and BRCA2 mutations. Exemplary such cancers include, without limitations, BRCA1-mutated breast cancer, BRCA-2 mutated cancer, BRCA-1 mutated ovarian cancer, BRCA-2 mutated prostate cancer, BRCA-mutated pancreatic cancer, fallopian tube cancer, peritoneal cancer, male breast cancer, and melanoma, as well as metastases thereof.

According to some of these embodiments, the therapeutically active agent is a PARP inhibitor, as described herein.

According to some of these embodiments, the composition comprises two or more therapeutically active agents, as described herein in any of the respective embodiments, wherein one is a PARP inhibitor. In some of these embodiments, another therapeutically active agent act in synergy, or provides an additive activity, when combined with the PARP inhibitor.

In exemplary embodiments, the other therapeutically active agent is a PD1 and/or PDL1 inhibitor, as described herein in any of the respective embodiments and any combination thereof, including a PD1 and/or PDL1 inhibitor which is an agent that interferes with an activity or expression of PD1 and/or PDL1, and/or an agent that interferes with an interaction between PD1 and PDL1 as described herein.

In exemplary embodiments, the other therapeutically active agent is a cytotoxic agent as described herein in any of the respective embodiments.

In exemplary embodiments, the other therapeutically active agent is a topoisomerase 1 inhibitor (TOP1 inhibitor or TOP1i), as described herein in any of the respective embodiments, for example, irinotecan, or any other member of the camptothecin family, including, for example, topotecan, camptothecin, SN-38, lurtotecan, exatecan mesylate, and rubitecan.

According to some of any of the embodiments described herein, the medical condition is treatable by a topoisomerase 1 inhibitor, and the one more of therapeutically active agents comprise a topoisomerase 1 inhibitor, as described herein. Such medical conditions include, without limitation, colorectal cancer, lung cancer (e.g., small cell lung cancer), cervical cancer, breast cancer, pancreatic cancer, GIST, and neuroendocrine tumors.

According to some of these embodiments, the composition comprises two or more therapeutically active agents, wherein one is a TOP1 inhibitor as described herein and the other is an agent that acts in synergy or additively therewith, for example, a PARP inhibitor, or any other anti-cancer as described herein. According to some of any of the embodiments described herein, the one or more therapeutically active agent(s) can be, independently, hydrophobic, hydrophilic or amphiphilic.

In some embodiments, the therapeutically active agent(s) comprises a hydrophobic therapeutically active agent(s).

In some embodiments, the therapeutically active agent(s) comprise a hydrophilic and/or amphiphilic therapeutically active agent(s).

In some embodiments, the therapeutically active agent(s) do not comprise a hydrophobic therapeutically active agent.

According to some of any of the embodiments described herein, the composition is a pharmaceutical composition that further comprises a pharmaceutical acceptable carrier.

According to some of any of the embodiments described herein, the (e.g., pharmaceutical) composition is usable in, or is for use in, treating the medical condition as described herein in a subject in need thereof.

According to some of any of the embodiments described herein, there is provided a process of preparing the composition of the present embodiments, which is effected essentially as described herein (see, e.g., the Examples section that follows).

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

In the context of these embodiments, an “active ingredient” refers to the agent or moiety accountable for the biological effect, that is, the particles having a therapeutically active agent associated therewith.

Hereinafter, the phrases “physiologically acceptable carrier” and “pharmaceutically acceptable carrier”, which may be interchangeably used, refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences”, Mack Publishing Co., Easton, PA, latest edition, which is incorporated herein by reference.

Suitable routes of administration may, for example, include oral, rectal, topical, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For topical administration, an appropriate carrier may be selected and optionally other ingredients that can be included in the composition, as is detailed herein. Hence, the compositions can be, for example, in a form of a cream, an ointment, a paste, a gel, a lotion, and/or a soap.

Ointments are semisolid preparations, typically based on vegetable oil (e.g., shea butter and/or cocoa butter), petrolatum or petroleum derivatives. As with other carriers or vehicles, an ointment base should be inert, stable, nonirritating and non-sensitizing.

Lotions are preparations that may to be applied to the skin without friction. Lotions are typically liquid or semiliquid preparations with a water or alcohol base, for example, an emulsion of the oil-in-water type. Lotions are typically preferred for treating large areas (e.g., as is frequently desirable for sunscreen compositions), due to the ease of applying a more fluid composition.

Creams are viscous liquids or semisolid emulsions, either oil-in-water or water-in-oil. Cream bases typically contain an oil phase, an emulsifier and an aqueous phase. The oil phase, also called the “lipophilic” phase, optionally comprises petrolatum and/or a fatty alcohol such as cetyl or stearyl alcohol. The aqueous phase optionally contains a humectant. The emulsifier in a cream formulation is optionally a nonionic, anionic, cationic or amphoteric surfactant.

Herein, the term “emulsion” refers to a composition comprising liquids in two or more distinct phases (e.g., a hydrophilic phase and a lipophilic phase). Non-liquid substances (e.g., dispersed solids and/or gas bubbles) may optionally also be present.

As used herein and in the art, a “water-in-oil emulsion” is an emulsion characterized by an aqueous phase which is dispersed within a lipophilic phase.

As used herein and in the art, an “oil-in-water emulsion” is an emulsion characterized by a lipophilic phase which is dispersed within an aqueous phase.

Pastes are semisolid dosage forms which, depending on the nature of the base, may be a fatty paste or a paste made from a single-phase aqueous gel. The base in a fatty paste is generally petrolatum, hydrophilic petrolatum, and the like. The pastes made from single-phase aqueous gels generally incorporate carboxymethylcellulose or the like as a base.

Gel formulations are semisolid, suspension-type systems. Single-phase gels optionally contain organic macromolecules distributed substantially uniformly throughout the carrier liquid, which is typically aqueous; but also, preferably, contains a non-aqueous solvent, and optionally an oil. Preferred organic macromolecules (e.g., gelling agents) include crosslinked acrylic acid polymers such as the family of carbomer polymers, e.g., carboxypolyalkylenes, that may be obtained commercially under the trademark Carbopol®. Other types of preferred polymers in this context are hydrophilic polymers such as polyethylene oxides, polyoxyethylene-polyoxypropylene copolymers and polyvinyl alcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

A composition formulated for topical administration may optionally be present in a patch, a swab, a pledget, and/or a pad.

Dermal patches and the like may comprise some or all of the following components: a composition to be applied (e.g., as described herein); a liner for protecting the patch during storage, which is optionally removed prior to use; an adhesive for adhering different components together and/or adhering the patch to the skin; a backing which protects the patch from the outer environment; and/or a membrane which controls release of a drug to the skin.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinylpyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water-based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., melanoma) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See, for example, Fingl et al., 1975, in “The Pharmacological Basis of Therapeutics”, Ch. 1, p. 1).

Dosage amount and interval may be adjusted individually to provide levels of the active ingredient sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is described herein.

According to some of any of the embodiments described herein, a composition as described herein in any of the respective embodiments is usable, or is for use, in treating a medical condition as described herein in any of the respective embodiments and any combination thereof, in a subject in need thereof.

According to some of any of the embodiments described herein, there is provided a use of a composition as described herein in any of the respective embodiments in the manufacture of a medicament for treating a medical condition as described herein in any of the respective embodiments and any combination thereof, in a subject in need thereof.

According to some of any of the embodiments described herein, there is provided a method of treating a medical condition as described herein in any of the respective embodiments and any combination thereof in a subject in need thereof, which comprises administering to the subject a composition as described herein in any of the respective embodiments.

The therapeutically agents or any combination of two or more therapeutically active agents are selected as suitable for use in treating the medical condition with which a subject is afflicted.

In some embodiments of any one of the embodiments described herein relating to treatment of a medical condition such as cancer (e.g., a SELP-expressing cancer), the treatment can further comprise administering at least one additional anti-cancer agent (i.e., in addition to the composition of the present embodiments) and/or anti-cancer therapy, including radiotherapy, chemotherapy, phototherapy and photodynamic therapy, surgery, nutritional therapy, ablative therapy, combined radiotherapy and chemotherapy, brachiotherapy, proton beam therapy, targeted therapy (e.g., BRAFi, MEKi, EGFRi, etc.), immunotherapy, cellular therapy and photon beam radiosurgical therapy. Analgesic agents and other treatment regimens are also contemplated. The additional treatment or therapy can be administered prior to, concomitant with, or subsequent to, the administration of a composition as described herein.

For example, the treatment can comprise administering a composition as described herein, which comprises a therapeutically active agent, along with an additional therapeutically active agent that do not form a part of the composition. The composition and the additional agent can form a part of the same pharmaceutical composition, or can be administered separately. Any combination of two or more therapeutically active agents, such as described herein, is contemplated.

For example, the treatment can comprise radiotherapy, which is administered prior to or concomitant with administering a composition as described herein. Such embodiments are useful in cases where SELP-expression is associated with irradiation, for example, in cases of irradiation-induced SELP expression.

According to some of any of the embodiments described herein, the medical condition is inflammation that is associated with overexpression of P-selectin.

The term “inflammation” as used herein refers to the general term for local accumulation of fluids, plasma proteins, and white blood cells initiated by physical injury, infection, or a local immune response. Inflammation may be associated with several signs e.g. redness, pain, heat, swelling and/or loss of function. Inflammation is an aspect of many diseases and disorders, including but not limited to diseases related to immune disorders, viral and bacterial infection, arthritis, autoimmune diseases, collagen diseases, allergy, asthma, pollinosis, and atopy (as described in further detail below).

Thus, inflammation can be triggered by injury, for example injury to skin, muscle, tendons, or nerves. Inflammation can be triggered as part of an immune response, e.g., pathologic autoimmune response. Inflammation can also be triggered by infection, where pathogen recognition and tissue damage can initiate an inflammatory response at the site of infection.

Inflammation according to the present teachings may be associated with chronic (long term) inflammatory diseases or disorders or acute (short term) inflammatory diseases or disorders.

According to some embodiments, the inflammation is associated with a disease selected from the group consisting of an infectious disease, an autoimmune disease, a hypersensitivity associated inflammation, a graft rejection and an injury.

Inflammation may be triggered by various kinds of injuries to muscles, tendons or nerves. Thus, for example, inflammation may be caused by repetitive movement of a part of the body i.e. repetitive strain injury (RSI). Diseases characterized by inflammation triggered by RSI include, but are not limited to, bursitis, carpal tunnel syndrome, Dupuytren's contracture, epicondylitis (e.g. tennis elbow), ganglion (i.e. inflammation in a cyst that has formed in a tendon sheath, usually occurring on the wrist), rotator cuff syndrome, tendinitis (e.g., inflammation of the Achilles tendon), tenosynovitis, and trigger finger (inflammation of the tendon sheaths of fingers or thumb accompanied by tendon swelling).

Many diseases related to infectious diseases include inflammatory responses, where the inflammatory responses are typically part of the innate immune system triggered by the invading pathogen. Inflammation can also be triggered by physical (mechanical) injury to cells and tissues resulting from the infection. Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases. According to one embodiment, examples of infections characterized by inflammation include, but are not limited to, encephalitis; meningitis; encephalomyelitis; viral gastroenteritis; viral hepatitis.

Furthermore, many immune disorders include acute or chronic inflammation. For example, arthritis is considered an immune disorder characterized by inflammation of joints, but arthritis is likewise considered an inflammatory disorder characterized by immune attack on joint tissues.

Inflammation according to the present teachings may be associated with a deficient immune response (e.g., HIV, AIDS) or with an overactive immune response (e.g., allergy, autoimmune disorders). Thus, inflammation according to the present teachings may be associated with any of the following:

Inflammatory Diseases Associated with Hypersensitivity:

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

### Type I or Immediate Hypersensitivity, Such as Asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann NY Acad Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost.2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like β-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann NY Acad Sci 1997 Dec. 29; 830:266), skin diseases, cutaneous diseases, dermal diseases, bullous skin diseases, Pemphigus vulgaris, bullous pemphigoid and Pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tₕ1 lymphocyte mediated hypersensitivity and Tₕ2 lymphocyte mediated hypersensitivity.

### Autoimmune Diseases:

Autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost.2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome.

Additional diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, Pemphigus vulgaris, bullous pemphigoid and Pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann NY Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann NY Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

According to one embodiment, the autoimmune disease is Crohn's disease, psoriasis, scleroderma or rheumatoid arthritis.

### Graft Rejection Diseases:

Examples of diseases associated with transplantation of a graft include, but are not limited to, graft rejection, chronic graft rejection, subacute graft rejection, hyperacute graft rejection, acute graft rejection and graft versus host disease.

### Allergic Diseases:

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

A person skilled in the art would know how to identify a medical condition associated with over expression of P-selectin, and a suitable therapeutically active agent, or a combination of two or more therapeutically active agents, or a combination with a therapeutically active agent and an additional therapy, for treating the medical condition, and can accordingly select a suitable composition according to the present embodiments.

As used herein, the term “subject” includes mammals, preferably human beings at any age which suffer from the pathology. Preferably, this term encompasses individuals who are at risk to develop the pathology.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

Herein, by “associated with” it is meant that one or more therapeutically active agents are in contact with the polymeric matrix, and/or bound to the polymeric matrix by physical and/or chemical interactions, for example, by covalent and/or non-covalent bonding (e.g., hydrogen bonds, van der Waals bonds, electrostatic interaction, and/or hydrophobic interaction). In some embodiments, one or more of the therapeutically active agents are physically associated with the polymeric matrix, for example, are embedded, entrapped or encapsulated by the polymeric matrix, and/or are entangled within or absorbed to the polymeric matrix.

Herein, the phrase “linking group” describes a group (e.g., a substituent) that is attached to two or more moieties in the compound; whereas the phrase “end group” or “terminal group” describes a group (e.g., a substituent) that is attached to a single moiety in the compound via one atom thereof.

Herein, the term “hydrocarbon” describes an organic moiety that includes, as its basic skeleton, a chain of carbon atoms, substituted mainly by hydrogen atoms. The hydrocarbon can be saturated or non-saturated, be comprised of aliphatic, alicyclic or aromatic moieties, and can optionally be substituted by one or more substituents (other than hydrogen). A substituted hydrocarbon may have one or more substituents, whereby each substituent group can independently be, for example, alkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, oxo, cyano, nitro, azo, azide, sulfonamide, carboxy, thiocarbamate, urea, thiourea, carbamate, amide, and hydrazine. The hydrocarbon can be an end group or a linking group, as these terms are defined herein. The hydrocarbon moiety is optionally interrupted by one or more heteroatoms, including, without limitation, one or more oxygen, nitrogen and/or sulfur atoms, and/or by one or more linking groups or bonds as described herein. As used herein throughout, the term “alkyl” refers to any saturated aliphatic hydrocarbon including straight chain and branched chain groups. Preferably, the alkyl group has 1 to 20 carbon atoms. Whenever a numerical range; e.g., “1 to 20”, is stated herein, it implies that the group, in this case the hydrocarbon, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or non-substituted. When substituted, the substituent group can be, for example, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

Herein, the term “alkenyl” describes an unsaturated aliphatic hydrocarbon comprise at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or non-substituted. Substituted alkenyl may have one or more substituents, whereby each substituent group can independently be, for example, alkynyl, cycloalkyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

Herein, the term “alkynyl” describes an unsaturated aliphatic hydrocarbon comprise at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted. Substituted alkynyl may have one or more substituents, whereby each substituent group can independently be, for example, cycloalkyl, alkenyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

The term “alkylene” describes a saturated or unsaturated aliphatic hydrocarbon linking group, as this term is defined herein, which differs from an alkyl group (when saturated) or an alkenyl or alkynyl group (when unsaturated), as defined herein, only in that alkylene is a linking group rather than an end group.

A “cycloalkyl” group refers to a saturated on unsaturated all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. When a cycloalkyl group is unsaturated, it may comprise at least one carbon-carbon double bond and/or at least one carbon-carbon triple bond. The cycloalkyl group can be an end group, as this phrase is defined herein, wherein it is attached to a single adjacent atom, or a linking group, as this phrase is defined herein, connecting two or more moieties.

An “aryl” group refers to an all-carbon monocyclic or fused-ring polycyclic (i.e., rings which share adjacent pairs of carbon atoms) end groups having a completely conjugated pi-electron system. Examples, without limitation, of aryl groups are phenyl, naphthalenyl and anthracenyl. The aryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroaryl” group refers to a monocyclic or fused ring (i.e., rings which share an adjacent pair of atoms) end group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

The term “arylene” describes a monocyclic or fused-ring polycyclic linking group, as this term is defined herein, and encompasses linking groups which differ from an aryl or heteroaryl group, as these groups are defined herein, only in that arylene is a linking group rather than an end group.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. When substituted, the substituted group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. Representative examples are piperidine, piperazine, tetrahydrofuran, tetrahydropyran, morpholine and the like. The heteroalicyclic group can be an end group, as this phrase is defined herein, wherein it is attached to a single adjacent atom, or a linking group, as this phrase is defined herein, connecting two or more moieties.

Herein, the terms “amine” and “amino” each refer to either a —NR′R″ group or a —N⁺R′R″R′″ end group, wherein R′, R″ and R′″ are each hydrogen or a substituted or non-substituted alkyl, alkenyl, alkynyl, cycloalkyl, heteroalicyclic (linked to amine nitrogen via a ring carbon thereof), aryl, or heteroaryl (linked to amine nitrogen via a ring carbon thereof), as defined herein. Optionally, R′, R″ and R′″ are hydrogen or alkyl comprising 1 to 4 carbon atoms. Optionally, R′ and R″ (and R′″, if present) are hydrogen. Herein, the terms “amine” and “amino” also refer to a —NR′— linking group, with R′ as defined herein.

An “azide” group refers to a —N═N⁺═N— end group.

An “alkoxy” group refers to any of an —O-alkyl, —O-alkenyl, —O-alkynyl, —O-cycloalkyl, and —O-heteroalicyclic end group, as defined herein, or to any of an —O-alkylene, —O-cycloalkyl- and —O-heteroalicyclic-linking group, as defined herein.

An “aryloxy” group refers to both an —O-aryl and an —O-heteroaryl group, as defined herein, or to an —O-arylene.

A “hydroxy” group refers to a —OH group.

A “thiohydroxy” or “thiol” group refers to a —SH group.

A “thioalkoxy” group refers to any of an —S-alkyl, —S-alkenyl, —S-alkynyl, —S-cycloalkyl, and —S-heteroalicyclic end group, as defined herein, or to any of an —S-alkylene-, —S-cycloalkyl- and —S-heteroalicyclic-linking group, as defined herein.

A “thioaryloxy” group refers to both an —S-aryl and an —S-heteroaryl group, as defined herein, or to an —S-arylene.

A “carbonyl” or “acyl” group refers to a —C(═O)—R′ end group, where R′ is defined as hereinabove, or to a —C(═O)— linking group or bond.

A “thiocarbonyl” group refers to a —C(═S)—R′ end group, where R′ is as defined herein, or to a —C(═S)— linking group or bond.

A “carboxy”, “carboxyl”, “carboxylic” or “carboxylate” group refers to both “C-carboxy” and “O-carboxy” end groups, as defined herein, as well as to a carboxy linking group, as defined herein.

A “C-carboxy” group refers to a —C(═O)—O—R′ group, where R′ is as defined herein.

An “O-carboxy” group refers to an R′C(═O)—O— group, where R′ is as defined herein.

A “carboxy linking group” refers to a —C(═O)—O— linking group or a carboxylate bond.

An “oxo” group refers to a ═O end group.

An “imine” group refers to a ═N—R′ end group, where R′ is as defined herein, or to an ═N-linking group or imine bond.

An “oxime” group refers to a ═N—OH end group.

A “hydrazone” group refers to a ═N—NR′R″ end group, where each of R′ and R″ is as defined herein, or to a ═N—NR′— linking group where R′ is as defined herein.

A “halo” group refers to fluorine, chlorine, bromine or iodine.

A “sulfinyl” group refers to an —S(═O)—R′ end group, where R′ is as defined herein, or to an —S(═O)— linking group.

A “sulfonyl” group refers to an —S(═O)₂—R′ end group, where R′ is as defined herein, or to an —S(═O)₂— linking group.

A “sulfonate” group refers to an —S(═O)₂—O—R′ end group, where R′ is as defined herein, or to an —S(═O)₂—O— linking group.

A “sulfate” group refers to an —O—S(═O)₂—O—R′ end group, where R′ is as defined as herein, or to an —O—S(═O)₂—O— linking group.

A “sulfonamide” or “sulfonamido” group encompasses both S-sulfonamido and N-sulfonamido end groups, as defined herein, as well as a sulfonamide linking group, as defined herein.

An “S-sulfonamido” group refers to a —S(═O)₂—NR′R″ end group, with each of R′ and R″ as defined herein.

An “N-sulfonamido” group refers to an R'S(═O)₂—NR″— end group, where each of R′ and R″ is as defined herein.

A “sulfonamide linking group” refers to a —S(═O)₂—NR′— linking group, where R′ is as defined herein.

A “carbamyl” or “carbamate” group encompasses both O-carbamyl and N-carbamyl end groups, as defined herein, as well as a carbamyl linking group or carbamate linking group, as defined herein.

An “O-carbamyl” group refers to an —OC(═O)—NR′R″ end group, where each of R′ and R″ is as defined herein.

An “N-carbamyl” group refers to an R′OC(═O)—NR″— end group, where each of R′ and R″ is as defined herein.

A “carbamyl linking group” or “carbamate” linking group refers to a —OC(═O)—NR′-linking group or bond, where R′ is as defined herein.

A “thiocarbamyl” or “thiocarbamate” group encompasses O-thiocarbamyl, S-thiocarbamyl and N-thiocarbamyl end groups, as defined herein, as well as a thiocarbamyl or thiocarbamate linking group, as defined herein.

An “O-thiocarbamyl” group refers to an —OC(═S)—NR′R″ end group, where each of R′ and R″ is as defined herein.

An “N-thiocarbamyl” group refers to an R′OC(═S)NR″— end group, where each of R′ and R″ is as defined herein.

An “S-thiocarbamyl” group refers to an —SC(═O)—NR′R″ end group, where each of R′ and R″ is as defined herein.

A “thiocarbamyl linking group” refers to a —OC(═S)—NR′— or —SC(═O)—NR′— linking group, where R′ is as defined herein.

An “amide” or “amido” group encompasses C-amido and N-amido end groups, as defined herein, as well as an amide linking group, as defined herein.

A “C-amido” group refers to a —C(═O)—NR′R″ end group, where each of R′ and R″ is as defined herein.

An “N-amido” group refers to an R′C(═O)—NR″— end group, where each of R′ and R″ is as defined herein.

An “amide linking group” refers to a —C(═O)—NR′— linking group or bond, where R′ is as defined herein.

A “urea group” refers to an —N(R′)—C(═O)—NR″R′″ end group, where each of R′, R″ and R″ is as defined herein, or an —N(R′)—C(═O)—NR″— linking group or bond, where each of R′ and R″ is as defined herein.

A “thiourea group” refers to an —N(R′)—C(═S)—NR″R′″ end group, where each of R′, R″ and R″ is as defined herein, or an —N(R′)—C(═S)—NR″— linking group or bond, where each of R′ and R″ is as defined herein.

A “nitro” group refers to an —NO₂ group.

A “cyano” group refers to a —C≡N group.

The term “phosphonyl” or “phosphonate” describes a —P(═O)(OR′)(OR″) group, with R′ and R″ as defined herein, or a —P(═O)(OR′)—O— linking group, with R′ as defined herein.

The term “phosphate” describes an —O—P(═O)(OR′)(OR″) end group, with each of R′ and R″ as defined herein, or —O—P(═O)(OR′)—O— linking group, with R′ as defined herein.

The term “phosphinyl” describes a —PR′R″ end group, with each of R′ and R″ as defined herein, or a —PR′— linking group, with R′ as defined herein.

The term “hydrazine” describes a —NR′—NR″R′″ end group, where R′, R″, and R′″ are as defined herein, or to a —NR′—NR″— linking group, where R′ and R″ are as defined herein.

As used herein, the term “hydrazide” describes a —C(═O)—NR′—NR″R′″ end group, where R′, R″ and R′″ are as defined herein, or to a —C(═O)—NR′—NR″— linking group, where R′ and R″ are as defined herein.

As used herein, the term “thiohydrazide” describes a —C(═S)—NR′—NR″R′″ end group, where R′, R″ and R′″ are as defined herein, or to a —C(═S)—NR′—NR″— linking group, where R′ and R″ are as defined herein.

A “guanidinyl” group refers to an RaNC(═NRd)—NRbRc end group, where each of Ra, Rb, Rc and Rd can be as defined herein for R′ and R″, or to an —R′NC(═NR″)—NR′″— linking group, where R′, R″ and R′″ are as defined herein.

A “guanyl” or “guanine” group refers to an R′″R″NC(═NR′)— end group, where R′, R″ and R′″ are as defined herein, or to a —R″NC(═NR′)— linking group, where R′ and R″ are as defined herein.

As used herein, the term “alkylene glycol” describes a —O—[(CR′R″)_{z}—O]Y—R′″ end group or a —O—[(CR′R″)_{z}—O]_{y}— linking group, with R′, R″ and R′″ being as defined herein, and with z being an integer of from 1 to 10, preferably, from 2 to 6, more preferably 2 or 3, and y being an integer of 1 or more. Preferably R′ and R″ are both hydrogen. When z is 2 and y is 1, this group is ethylene glycol. When z is 3 and y is 1, this group is propylene glycol. When y is 2-4, the alkylene glycol is referred to herein as oligo(alkylene glycol).

When y is greater than 4, the alkylene glycol is referred to herein as poly(alkylene glycol). In some embodiments of the present invention, a poly(alkylene glycol) group or moiety can have from 10 to 200 repeating alkylene glycol units, such that z is 10 to 200, preferably 10-100, more preferably 10-50.

For any of the embodiments described herein, any of the moieties described herein, and particularly the SELP-selective targeting moiety of, e.g., Formula I, as described herein, may be in a form of a salt, for example, a pharmaceutically acceptable salt.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent moiety, while not abrogating the biological activity and properties of the moiety. A pharmaceutically acceptable salt of a targeting moiety as described herein can alternatively be formed during the synthesis of the particle, e.g., in the course of isolating the particle from a reaction mixture or re-crystallizing the particle. In the context of some of the present embodiments, a pharmaceutically acceptable salt of the targeting moiety can be a salt of the moiety of Formula I, in which R is H, and the salt is an ionized form of the respective sulfate or sulfonate moiety, and a counter ion. The counter ion can be, for example, a monovalent cation (monocation), in a stoichiometric amount with respect to the number of ionized groups in the targeting moiety, or, in case there are two or more ionized groups, a divalent, tri-valent, etc., cation. The cation can be, for example, a metal cation, for example, monovalent cations of sodium, potassium etc., or divalent cations of magnesium, calcium, etc. Any other metal cations are contemplated.

In the context of some of the present embodiments, a pharmaceutically acceptable salt of the targeting moieties described herein may optionally be an acid addition salt and/or a base addition salt.

An acid addition salt comprises at least one basic (e.g., amine and/or guanidinyl) group of the compound which is in a positively charged form (e.g., wherein the basic group is protonated), in combination with at least one counter-ion, derived from the selected acid, that forms a pharmaceutically acceptable salt. The acid addition salts of the compounds described herein may therefore be complexes formed between one or more basic groups of the compound and one or more equivalents of an acid.

A base addition salt comprises at least one acidic (e.g., carboxylic acid) group of the compound which is in a negatively charged form (e.g., wherein the acidic group is deprotonated), in combination with at least one counter-ion, derived from the selected base, that forms a pharmaceutically acceptable salt. The base addition salts of the compounds described herein may therefore be complexes formed between one or more acidic groups of the compound and one or more equivalents of a base.

Depending on the stoichiometric proportions between the charged group(s) in the compound and the counter-ion in the salt, the acid additions salts and/or base addition salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof, and/or a carboxylate anion and a base addition salt thereof.

The base addition salts may include a cation counter-ion such as sodium, potassium, ammonium, calcium, magnesium and the like, that forms a pharmaceutically acceptable salt.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

Further, each of the moieties, groups and/or particles as described herein, including the salts thereof, can be in a form of a solvate or a hydrate thereof.

The term “solvate” refers to a complex of variable stoichiometry (e.g., di-, tri-, tetra-, penta-, hexa-, and so on), which is formed by a solute (the heterocyclic compounds described herein) and a solvent, whereby the solvent does not interfere with the biological activity of the solute.

The term “hydrate” refers to a solvate, as defined hereinabove, where the solvent is water.

The moieties, groups and/or particles (structures) described herein encompass any stereoisomer, including enantiomers and diastereomers, of the moieties and/or groups described herein, unless a particular stereoisomer is specifically indicated.

As used herein, the term “enantiomer” refers to a stereoisomer of a compound or moiety that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an (R) or an (S) configuration and any combination, and compounds according to some embodiments of the present invention, can have any their chiral centers exhibit an (R) or an (S) configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

### Materials:

PLGA (D,L-lactide-co-glycolide 85-15, ester terminated, Mw 50,000-70,000 Da), PLGA-PEG (PEG average Mw 2,000 Da, PLGA average Mw 11,500 Da), PVA (Poly(vinyl alcohol), Mw 13,000-23,000 Da, 87-89% hydrolyzed), PEG (Poly(ethylene glycol), Mw 2,000 Da), PLGA (Poly(D,L-lactide-co-glycolide), 50-50 Mw 7,000-17,000 Da, Resomer® 502—ester terminated or Resomer® 502H, acid terminated) were obtained from Sigma-Aldrich, Rehovot, Israel. Dialysis tubes: mini-GeBaFlex tube 200 μL, 8 kDa MWCO, or Mega GeBaFlex tubes 20 mL, 3.5 kDa MWCO, were obtained from Gene Bio-Application Ltd, Yavne, Israel. Dabrafenib 98% purity was obtained from Tzamal-D-chem, Petach-Tikva, Israel. Trametinib 98% purity was obtained from Advanced ChemBlocks Inc., CA, USA; 2,2-dimethoxy-2-phenylacetophenone (DMPA, 99%) and 1-thioglycerol were obtained from Sigma-Aldrich. HO-PLGA₁₂ₖ-PEG₂ₖ-allyl (polymer 11) was obtained from ‘Creative PEGWorks’.

All chemical reagents, including salts and solvents, were AR grade and were obtained from Sigma-Aldrich, unless mentioned otherwise.

Chemical reactions requiring anhydrous conditions were performed under Argon atmosphere.

Distilled water, filtered using a Milli-Q® purification system, was used throughout.

### Methods:

PLGA Nanoparticles preparation: PLGA NPs were prepared using a benchtop NanoAssemblr® instrument (Precision NanoSystems Inc., Vancouver, Canada). PLGA and PLGA-PEG were dissolved in acetonitrile (ACN) at a 2:1 ratio, to a final polymer concentration of 6 mg/mL.

PLGA-PEG-Glycerol-(SO₃)₂ NPs synthesis: The NPs were synthesized using a benchtop NanoAssemblr® instrument (Precision NanoSystems Inc., Vancouver, Canada), using PLGA (lactide:glycolide 85:15 (LACTEL®; MW 66,000-107,000; Sigma), PLGA-PEG (PEG average MW 2,000 PLGA average MW 11,500 Da; Sigma), or PLGA-PEG-Glycerol-(SO₃)₂ at 2:1 ratio, respectively. The polymers were dissolved in acetonitrile to a final polymer concentration of 6 mg/mL.

Drug-loaded (TRM and DBF) PLGA Nanoparticles preparation: TRM (2 mg/mL) and DBF (4.5 mg/mL) were dissolved in ACN at a 1:10 ratio and added to the polymer solution. PLGA NPs were formed by nanoprecipitation achieved by mixing two fluid phases: (1) organic phase comprised of polymers and drug solutions (2) aqueous phase comprised of 2.5% w/v PVA solution. The aqueous and the organic phases were mixed at a 12 mL/minute flow rate and a 2:1 ratio, respectively. Then, ACN was evaporated under reduced pressure using a rotary evaporator, and the NPs were centrifuge (35,000 g, 30 minutes) and washed twice with 5 ml Milli-Q® water. Finally, the NPs were re-suspended in PBS or Milli-Q® water.

Drug-loaded (talazoparib (PARPi) and PDL1i) PLGA Nanoparticles preparation: Talazoparib (4.5 mg/mL) and exemplary PDL1i SM56 (18 mg/mL) or SM69 (18 mg/mL) were dissolved in acetonitrile or DMSO, respectively, and added to the polymer solution. The NPs were formed by nanoprecipitation achieved by mixing two fluid phases:
(1) organic phase comprised of polymers and drugs in acetonitrile solution.
(2) aqueous phase comprised of 2.5 w/v % PVA solution.

The aqueous and the organic phases were mixed at a 12 mL/minute flow rate at 2:1 ratio. Subsequently, acetonitrile was evaporated under reduced pressure using a rotary evaporator, and the NPs were centrifuge and washed with 5 mL DDW twice (35,000 g, 30 minutes). The NPs were re-suspended in PBS, DDW or lyophilized in 5% trehalose solution.

Drug-loaded (talazoparib (PARPi) and TOP1i) PLGA Nanoparticles preparation: For the co-encapsulation of talazoparib with a TOP1 inhibitor (e.g., irinotecan, topotecan) or ATR inhibitor (e.g., berzosertib), the compounds were first dissolved in acetonitrile, then the drug combination was added to the polymers solution at 1:10 ratio, respectively, following nanoprecipitation in 2% PVA solution and downstream processing as described above.

X-ray photoelectron spectroscopy (XPS): In order to determine the PLGA-PEG-Glycerol-(SO₃)₂ NPs elemental composition and to determine that the sulfate groups are present in the surface of the nanoparticles XPS, a surface-sensitive and quantitative analysis was performed. Samples were prepared depositing 60 μL of freshly prepared PLGA-PEG-Glycerol-(SO₃)₂ NPs in silica wafers. The samples were covered with a petri dish to avoid any contamination for 72 hours, until the complete evaporation of the drop. Prior to the spectral acquisition, surface cleaning was performed via sputtering with a 1000 Cluster Ar ion beam at an energy of 4 keV. The sputtering duration was set to 60 seconds, an interval selected to effectively remove surface contaminants without significantly altering the inherent surface chemistry of the sample. The analyses utilized an X-ray beam with a diameter of 650 μm under ultra-high vacuum conditions. The XPS system was equipped with a monochromatic Al Kα X-ray source to excite the electrons. Spectra were acquired using a pass energy appropriate for high resolution scans, with the exact value determined based on the equipment's specifications and the information depth required. Charge compensation was achieved through the use of a dual beam system. This system employs both low-energy electrons and a low-energy argon ion beam concurrently to neutralize the positive charge that can accumulate during XPS analysis. All measurements were performed at room temperature, and the data were calibrated against the C is peak at 284.8 eV if required, to compensate for any charging effects that were not neutralized by the dual beam system.

Nanoparticles size distribution and Zetapotential: Samples were freshly prepared in Milli-Q® water at 0.1 mg/mL NPs concentration. Mean hydrodynamic diameter, dispersity index (D index), and Zeta potential were measured using a dynamic light scattering (DLS) Möbius instrument at a 540 nm laser wavelength using a 532 nm long-pass filter (Wyatt Technology Corporation, Santa Barbara, CA 93117 USA). All measurements were performed at 25° C.

Colloidal stability: Freshly prepared PLGA NPs samples were incubated in PBS, pH 7.4, or in DMEM medium containing 10% FBS and maintained at 37° C. NPs size, D index, and Zeta potential were monitored by DLS for 48 hours. The experiments were repeated three times, and mean values and standard deviations (SD) were calculated.

Drug loading and encapsulation efficiency: Lyophilized drug-loaded NPs were dissolved in ACN and stirred at 60° C. for one hour. Samples were filtered through a Polyvinylidene Fluoride membrane filter with a 0.2 μm pore size and analyzed by HPLC. UltiMate® 3000 Nano LC systems (Dionex) was equipped with 3000 pump, VWD-3000 UV-Vis detector, and Chromeleon® 6.80 software. The column used was Jupiter column, particle size 5 μm, pore size 300 Å, 4.6×250 mm, C18 reversed-phase (RP). Chromatographic conditions: flow: 1.0 mL/minutes, gradient: 30% sol. B to 100% sol. B (solution A—0.1% trifluoroacetic acid (TFA) in water; solution B—0.1% TFA in acetonitrile (ACN)). The injection volume was 20 μL. Drug entrapment efficiency was calculated both as drug loading content (DLC w/w %), Equation (i), and drug loading efficiency (DLE %), Equation (ii):

$$DLC ( w / w \% ) = \frac{mass of drug in nanoparticles ( mg )}{PLGA nanoparticles weight ( mg )} \times 100 \qquad Equation ( i )$$

$$DLE ( \% ) = \frac{mass of drug in nanoparticles ( mg )}{total mass of drug used in formulation ( mg )} \times 100 \qquad Equation ( ii )$$

Release profile: Drug-loaded NPs were suspended in DDW, aliquoted (200 μL) into several semipermeable dialysis tubes (mini-GeBAFlex tube, 6-8 kDa MWCO, obtained from Gene Bio-Application Ltd, Yavne, Israel), and dialyzed against 1 Liter (L) of PBS (pH 7.4, 1:5000 dialysis ratio), or DMEM medium containing 10% FBS, at 37° C. At predetermined time points, for 48 hours, an aliquot of the NPs in suspension were subsequently removed, lyophilized, and dissolved in ACN. The amount of drug released was quantified by HPLC, as previously described in Kolishetti et al. [Proceedings of the National Academy of Sciences 107, 17939-17944 (2010)]. The experiments were repeated three times.

Transmission electron microscopy (TEM): PLGA NPs were visualized using 110 keV TEM (JEM-1400Plus Transmission Electron Microscope). TEM ultrathin Formvar-coated 200-mesh copper grids (Ted-pella, Inc.) were prepared by depositing a drop of the sample over the grid until drop evaporation.

In order to increase the resolution of the images, the samples were stained using the negative staining technique. Briefly, 24 hours after sample preparation, the grids were charged for 5 minutes with ultraviolet light, then one drop of Uranyl Acetate (staining agent) was deposited over a parafilm strip and the grid with the sample was deposited over the staining agent drop for 1 minute. Following, excess liquid was removed with filter paper and TEM images were acquired. For size distribution analysis, the size of at least 20 particles was measured and the average size and SD were obtained with ImageJ software.

¹H-NMR: Spectra were recorded on Bruker Avance I and Avance III 400 MHz/100 MHz spectrometers as indicated. Chemical shifts are reported in ppm and referenced to the solvent.

Size Exclusion Chromatography (SEC): PLGA-PEG-Glycerol (OH)₂ was characterized using Malvern Viscotek GPCmax equipped with 2×PSS GRAM 1000 Å+PSS GRAM 30 Å columns. The polymer was run with a mobile phase composed of dimethylformamide (DMF)+25 mM ammonium acetate (NH₄Ac) with a flow rate of 0.5 mL/minute with a runtime of 90 minutes. 50 μL from a 10 mg/ml sample polymer solution in the mobile phase was injected and before that the polymer solution was filtered with 0.45 μM PTFE filter. All other measurements were recorded on Viscotek GPCmax by Malvern using a refractive index detector and PEG standards (purchased from Sigma-Aldrich) were used for calibration.

For the SEC characterization of Polymer 14, PLGA-PEG-Glycerol-(SO₃Na)₂, Malvern Viscotek GPCmax equipped with 2×PSS GRAM 1000 Å was used. After a needle wash with DMF, the polymer was run with a mobile phase composed of DMF)+25 mM ammonium acetate (NH₄Ac) with a flow rate of 0.5 mL/minute with a runtime of 60 minutes. 50 μL from a 20 mg/mL sample amphiphiles solution in the mobile phase was injected and before that the polymer solution was filtered with 0.45 μM PTFE syringe filter. Column temperature: 50° C.; Detector: Viscotek VE3580 RI detector (see, FIG. 28B; PEG standards were used for calibration, and the Mn and P values of the amphiphiles were calculated accordingly).

Cell culture: Murine primary melanoma D4M.3A cells were obtained from Dartmouth College, Hanover. D4M.3A cells were cultured in Advanced DMEM growth media supplemented with 5% FBS, 100 μg/mL streptomycin, 100 IU/mL penicillin, 12.5 IU/mL nystatin, and 1% GlutaMAX™ (Gibco, Waltham, MA, USA). Human metastatic melanoma 131/4-5B1 cells were obtained from University of Toronto, Canada, and were cultured in RPMI 1640 medium supplemented with 10% FBS, 100 μg/mL streptomycin, 100 IU/mL penicillin, 12.5 IU/mL nystatin, and 2 mM L-Glutamine (Biological Industries Ltd, Kibbutz Beit Haemek, Israel). Human primary melanoma A375 cells were obtained from ATCC and were cultured in RPMI 1640 medium supplemented with 10% FBS, 100 μg/mL streptomycin, 100 IU/mL penicillin, 12.5 IU/mL nystatin, and 2 mM L-Glutamine. Human primary melanoma WM115 cells were obtained from ATCC and cultured in EMEM growth media (Gibco, Waltham, MA, USA) supplemented with 10% FBS, 100 μg/mL streptomycin, 100 IU/mL penicillin, 12.5 IU/mL nystatin, 1% of non-essential amino acids, 1% of sodium pyruvate and 2 mM L-glutamine. Human microvascular endothelial hCMEC/D3 cells were obtained from Merck and grown in EndoGRO™ MV Complete Media Kit (SCM004—Basal media+supplement kit) supplemented with 1 ng/mL FGF-2 (bFGF) (Merck, Germany). Human astrocytes were obtained from ScienCell™ and grown in the supplied astrocyte medium complemented with a supplemented kit (ScienCell™, CA, USA). Human brain pericytes and human microglia were obtained from ScienCell™ and grown in the supplied Pericyte Medium or Microglia medium complemented with a supplement kit (ScienCell™, CA, USA). All Cells were grown at 37° C. and 5% CO₂ and were routinely tested for mycoplasma contamination with a mycoplasma detection kit that was obtained from Biological Industries (Kibbutz Beit-HaEmek, Israel).

Human MDA-MB-231 and MDA-MB-436, and murine 4T1 and EMT6 cells were obtained from the ATCC. EMT6, MDA-MB-231 and MDA-MB-436 cells were cultured in DMEM supplemented with 10% FBS, 100 IU/mL penicillin, 100 μg/mL streptomycin, 12.5 U/mL Nystatin, 2 mM L-glutamine. 4T1 cells were cultured in RPMI supplemented with 10% FBS, 100 IU/mL Penicillin, 100 μg/mL Streptomycin, 12.5 U/mL Nystatin, 2 mM L-glutamine, 1 mM sodium pyruvate and 10 mM HEPES Buffer.

Proliferation assay: To evaluate the cytotoxicity of DBF and TRM combination, D4M.3A (7.5×10³ cells/well), 131/4-5B1 (15×10³ cells/well), and A375 (10×10³ cells/well) cell lines were plated onto a 24-well plate in growth media as indicated previously. Following 24 hours incubation, the cultured medium was replaced with a medium containing serial dilutions of DBF, TRM, and their combination as free or drug-loaded NPs. For the combined therapy, TRM concentration was 10 times more diluted than DBF concentration, which is equivalent to the ratio of the drugs in the NPs.

Untreated (control) cells were supplemented with fresh drug-free medium 24 hours after seeding. Following 72 hours incubation, the cells were counted using a Z1 Coulter Counter (Beckman Coulter). Each treatment was assayed in triplicates and the experiment was repeated three times. The proliferation of cells was normalized to the cell growth in the control group.

For MDA-MB-436, MDA-MB-23, 4T1 and EMT6, cells were seeded in 24-well plates (2.5×10⁴, 2.5×10⁴, 1×10⁴, 2×10³ cells/well, respectively) and incubated for 24 hours. Then, the cultured medium was replaced with a medium containing a serial dilution of talazoparib, talazoparib-loaded NPs or blank NPs (carrier only) for additional 72 hours. Untreated (control) cells were supplemented with fresh medium containing 0.1% DMSO. The cells were counted using a Z1 Coulter Counter (Beckman Coulter). Each treatment was assayed in triplicates, N=3.

Isobolograns and combination index: The interactions between DBF and TRM were evaluated by constructing isobolograms for different drugs ratios and calculating their combination index (CI).⁵¹ First, the inhibitory concentration (IC) values of treatment with DBF, TRM, and their combinations were calculated from the proliferation assays. Then, IC30, 50, 70 values of free TRM and DBF were marked on the X and Y axes, and the lines that represent additive effect were drawn between each IC. Data points in the upper-right of the IC line represent an antagonistic effect, while in the lower-left they represent a synergistic effect.

$$CI = \frac{{C}_{A , x}}{{IC}_{x , A}} + \frac{{C}_{B , x}}{{IC}_{x , B}}$$

Multicellular 3D tumor spheroids (MCTS) invasion assay: D4M.3A and WM115 mCherry-labeled 3D spheroids were prepared by the “hanging drop” method. WM115 spheroids contained 1000 cells/spheroid and D4M.3A spheroids contained 700 cells/spheroid. For MCTS, a mixture of human WM115 cells and brain resident cells (astrocytes, microglia, pericytes, and endothelial cells) was prepared at a 1:1:0.5:0.5:2 ratio. Each of the cells was labeled with a different fluorescent label, while the microglia cells were not labeled. The cells were deposited in 25 μL droplets composed of 0.24% w/v methylcellulose in medium and incubated on the inner side of a 20 mm dish for 72 hours at 37° C. The plates were placed upside down to allow the formation of 3D spheroids. Then, the spheroids were seeded in Matrigel® (BD, Franklin Lakes NJ, USA), in a 96-well plate (50 μl/well) and incubated for 1 hour at 37° C. Next, the spheroids were treated with free drugs or drug-loaded NPs, and their ability to invade the Matrigel® was imaged using the EVOS FL Auto cell imaging system (Thermo Fisher Scientific) and quantified with ImageJ software.

Hemolysis assay: Fresh blood was obtained from male Wistar rats by cardiac puncture and collected in heparinized tubes. The erythrocytes were washed three times with PBS and re-suspended to a final 2% w/v solution. The solution was then incubated with serial dilutions of PLGA NPs, (0.001-2 mg/mL), for 1 hour at 37° C. Dextran (Mw 70 kDa, Sigma-Aldrich, Rehovot, Israel) and PBS were used as negative controls whereas sodium dodecyl sulfate (SDS) was used as a positive control. Following centrifugation, the supernatants were transferred to a 96-well plate and the absorbance was measured at 550 nm using a SpectraMax® M5e plate reader (Molecular Devices LLC, Sunnyvale, California, USA). The results are normalized to the percentage of hemoglobin released by a 1% w/v of Triton×100 solution (100% lysis).

In studies on BRCA-mutated cells, fresh blood was obtained from C57/BL6 mice by cardiac puncture, collected in heparinized tubes and centrifuged at 1100 rpm for 5 minutes. The obtained red blood cells (RBCs) pellet was washed three times with 0.15 M NaCl, centrifuged and re-suspended in PBS to a final concentration of 2% w/w. RBCs were then incubated with serial dilutions of PLGA-PEG NPs (0.001-5.0 mg/mL) at 37° C. for 1 hour. Sodium dodecyl sulfate (SDS) was used as a positive control, and dextran (Mw 70 kDa, Sigma-Aldrich, Rehovot Israel) was used as a negative control (0.0001-1.0 mg/mL). Samples were centrifuged at 1100 rpm for 5 minutes and 100 μL of the supernatants were plated into a 96-well plate. Absorbance was analyzed at 550 nm using SpectraMax® M5e plate reader (Molecular Devices LLC, Sunnyvale, CA, USA). The degree of hemolysis is expressed as percent absorbance compared to treatment with 1% v/v of Triton X-100 solution (100% hemolysis).

In vivo motor coordination behavioral test: DBF- and TRM-related motor coordination was assessed by a Rotarod apparatus (Columbus Instruments, OH, USA). The test measures the mouse's ability to maintain itself on a rod that turns at an increasing speed. Mice were tested before treatment initiation and following the 7^{th} treatment of 1 or 2 mg/kg DBF and 0.1 or 0.2 mg/kg TRM as free or drug-loaded NPs, respectively. The control groups were treated with PBS or vehicle (0.01% DMSO, 83 mg/mL PEG 200, and 14 mg/mL Tween-80 in PBS). The initial speed was 1.6 rpm, with an acceleration rate of 4 rpm per minute. Animals were tested five times during each session, with at least 2 minutes of rest between each test. The three best performances of each mouse were considered, and the results were averaged for the whole group.

In vivo biodistribution and tumor accumulation: Male C57BL/6 mice were inoculated subcutaneously (SC) with 1×10⁶ D4M.3A cells at a dorsal site (upper back). Mice bearing about 230 mm³ tumors were treated intravenous (IV) with PLGA-Cy5 NPs or DBF-Cy5 at 20 μM Cy5 equivalent concentration. Tumor accumulation of Cy5 labeled DBF or NPs was monitored by CRI Maestro non-invasive intravital fluorescence imaging system. Mice were anesthetized using ketamine (100 mg/kg) and xylazine (12 mg/kg), shaved, and the accumulation of Cy5 signal was recorded 24 hours post-treatment. At 3 and 24 hours post-treatment mice were euthanized and the NPs accumulation in organs and tumors was calculated in ex vivo fluorescence images, in which the total fluorescence was divided by the organ area. Multispectral image-cubes were acquired through 590-750 nm spectral range in 10 nm steps using excitation (635 nm) and emission (675 nm) filter set. Mice auto-fluorescence and undesired background signals were eliminated by spectral analysis and the Maestro linear unmixing algorithm.

In studies on BRCA-mutated cells, female BALB/c mice were inoculated subcutaneously into the mammary fat pad with 0.2×10⁶ EMT6 cells. Mice bearing about 200 mm³ tumors were treated intravenous (IV) with Cy5 labelled PLGA-PEG NPs or PLGA-PEG-Glycerol-(SO₃)₂ at 20 μM Cy5 concentration. Tumor accumulation of the Cy5 signal was monitored by CRI Maestro non-invasive intravital fluorescence imaging system. Mice were anesthetized using ketamine (100 mg/kg) and xylazine (12 mg/kg), shaved, and the accumulation of Cy5 signal was recorded over time (0, 1, 2, 3, 6, and 24 hours post-treatment. At 3 and 24 h post-treatment mice were euthanized and the NPs accumulation in tumors was calculated ex vivo. NPs biodistribution was also monitored 24 hours post-injection. The total fluorescence normalized to organ/tumor area. Multispectral image-cubes were acquired through 590-750 nm spectral range in 10 nm steps using excitation (635 nm) and emission (675 nm) filter set. Mice auto-fluorescence and undesired background signals were eliminated by spectral analysis and the Maestro linear unmixing algorithm.

SELP expression by flow cytometry: P-selectin expression in 2D plated D4M.3A and WM115 cells was measured by flow cytometry. First, the cells were grown to high confluency in a 10 mm petri dish, then they were scraped with PBS, centrifuged (2000 RPM, 5 minutes, 4° C.), and re-suspended in 1 mL FACS buffer (PBS supplemented with 0.5% bovine serum albumin and 0.5 mM EDTA, Sigma-Aldrich).

For SELP expression in 3D spheroids, tumor spheroids were recovered from the Matrigel® using a cell recovery solution (BD, catalog no. 354253). The recovered cells were washed with PBS and centrifuged (2000 RPM, 5 minutes, 4° C.). Following, 0.5×10⁶ D4M.3A cells, were incubated with fluorescent-CD62p antibody (BD, FITC Rat anti-mouse, catalog no. 561923, 1:20 dilution) or with Isotype control (BD, FITC rat immunoglobulin G1 (IgG1), catalog no. 553995, 1:20 dilution) for 1 hour on ice. For WM115 cells (1×10⁶ cells) were incubated with primary mouse anti-human antibody (R & D Systems, catalog no. BBA1, 1:20 dilution) for 1 hour on ice or with an Isotype Control (R & D Systems, mouse isotype control, catalog no. mab002, 1:20 dilution). Then, the recovered cells were washed with PBS, centrifuged (2000 RPM, 5 minutes, 4° C.), and incubated with a secondary antibody, goat anti-mouse Alexa Fluor 647 (AbCAM, Ab150115, 1:300 dilution). For EMT6, the cells were washed with PBS, centrifuged (2000 rpm, 5 minutes) and re-suspended in 1 mL FACS buffer (PBS supplemented with 1% FBS, 0.1% NaN₃, and 0.5 mM EDTA). 1×10⁶ EMT6 cells were then incubated with fluorescent-CD62p antibody (BD, FITC Rat anti-mouse, #561923, 1:20 dilution) or with Isotype control (BD, FITC rat immunoglobulin G1 (IgG1), #553995, 1:20 dilution) for 1 hour on ice.

After incubation, the cells were washed with PBS twice and the fluorescent intensity was analyzed using Attune NxT Acoustic Focusing Flow Cytometer (Thermo Fisher Scientific, MA, USA). The results were analyzed by the Kaluza software.

SELP expression by immunohistochemistry (IHC): For immunostaining of murine melanoma tumors and brain metastases, tissues were embedded in an optimal cutting temperature (OCT) compound and cryo-sectioned into 5 μm thick sections. Immunostaining was performed using the BOND RX automated IHC Stainer (Leica Biosystems). Slides were fixated in cold acetone for 20 minutes and then incubated with 10% goat serum for 30 minutes to block non-specific binding sites. Next, the slides were incubated with mouse anti-mouse/rat SELP antibody (catalog no. 148301 Biolegend, 1:30 dilution) for 1 hour. Then, the slides were incubated with Mouse-IgG \hat{k} BP-CFL 488 (catalog no. sc-516176, Santa Cruz Biotechnology, 1:20 dilution) for an additional 1 hour, followed by Hoechst fluorescent dye (1:5000) for an additional 10 minutes for nuclei counterstaining. Tissues were later mounted with ProLong™ Gold antifade mountant (Thermo Fischer Scientific™) and imaged using the EVOS® FL Auto cell imaging system (Thermo Fisher Scientific). For human samples: Formalin-Fixed Paraffin-embedded slides (10 μm) were re-heated to 60° C. for 20 minutes and then incubated with 10% goat serum for 30 minutes to block non-specific binding sites. The slides were subsequently incubated with mouse anti-human E/P selectin (R & D, 1:30 dilution) for 1 hour, and then incubated with mouse-IgG k BP-CFL 488 (catalog no. sc-516176, Santa Cruz Biotechnology, 1:20 dilution) for an additional 1 hour, followed by Hoechst fluorescent dye (1:5000) for an additional 10 minutes for nuclei counterstaining. Tissues were then mounted with 70% ethanol for 1 minute, 95% ethanol for 1 minute, 100% ethanol for 1 minute, 100% ethanol for 1 minute, xylene for 1 minute, xylene for 1 minute, and with xylene based mounting (Thermo Fisher Scientific).

Cy5-NPs internalization into 3D spheroids: D4M.3A and WM115 spheroids were prepared by seeding the cells (1000 cells/spheroid for WM115 cells and 500 cells/spheroid for D4M.3A cells) in an ultra-low attachment round-bottom 96-well plate (Corning®) and allowing the cells to form spheroids for 96 hours. Followed, the medium was replaced with a fresh medium containing 1 μM Cy5-labeled targeted and non-targeted NPs. For NPs internalization following treatments with SELP inhibitor (SELPi), the spheroids were treated with SELPi (0.1, 1, and 10 μM) 1 hour before adding the NPs. The plate was then placed in the Incucyte® Live-cell analysis system (Essen Bioscience) and the Cy5 fluorescent intensity within the spheroids (brightfield) was monitored for 20-26 hours.

EMT6 spheroids were prepared by seeding the cells (500 cells/well/spheroid) in an ultra-low attachment round-bottom 96-well plate (Corning) and allowing them to form spheroids for 72 hours. After spheroids complete formation the medium was replaced with a fresh medium containing Cy5-labeled targeted and non-targeted NPs (1 μM Cy5). For NPs internalization assays following treatments with SELP inhibitor small molecule (SELPi), the spheroids were treated 10 μM SELPi for 1 hour before adding the NPs. The plate was placed in the IncuCyte Live cell analysis system (Essen Bioscience) and the Cy5 fluorescent intensity within the spheroids (brightfield) was monitored for 20 hours.

NPs binding to immobilized SELP: Recombinant human SELP (1 ng/μL, 50 μL per well), was incubated in a Nunc-Immuno 96 microwells plate (Sigma-Aldrich) at 4° C., overnight. Then, the wells were blocked with 3% skim milk solution (200 μL per well) for 1 h, washed three times with 200 μL PBS and incubated with Cy5-labeled PLGA-PEG-Glycerol-(SO₃)₂ NPs or PLGA-PEG NPs for 15 min at three different concentrations: 500, 300, 150 μg/mL. Next, the wells were wash three times with 200 μL PBS and Cy5 fluorescence signal was measured by a SpectraMax® M5e plate reader.

Animal studies: To evaluate the antitumor activity of drug-loaded NPs compared to free Drugs, 7-8 weeks old male C57BL/6 mice (Envigo C R S, Israel) were inoculated subcutaneous (SC) with 1×10⁶ D4M.3A cells. The tumors were allowed to establish for 10 days (tumor size of about 180 mm³) and then the mice were treated with DBF, TRM, or their combination as drug-loaded NPs or free drugs. Treatments were administered IV at 3 mg/kg DBF and 0.3 mg/kg TRM. Control groups were treated with PBS, Blank (empty) NPs, and vehicle (0.01% DMSO, 83 mg/mL PEG 200, and 14 mg/mL Tween-80 in PBS). Mice were treated every other day (QOD), and the tumors were measured by a digital caliper according to the formula: width²×length×0.52. Body-weight and tumor size were monitored every other day. After the 7^{th} treatment, the mice were euthanized, perfused with PBS and 4% formaldehyde, and the tumors were harvested. The tissues were then embedded in an OCT compound, and frozen-sectioned.

For drug dosing assessment, 1×10⁶ D4M.3A cells were inoculated SC into 7-8 weeks old C57BL/6 mice (Envigo C R S, Israel) and allowed to form tumors for 10 days (tumor size of about 70 mm³). Mice were then randomized into 6 groups and treated with IV injections of either free or drug-loaded NPs at 1 or 2 mg/kg DBF and 0.1 or 0.2 mg/kg TRM, QOD. The body-weight and tumor volume were monitored every other day and the mice were euthanized when tumor volume reached above 1000 mm³ or when mice lost more than 20% from their initial (day 0) body-weight. Blood samples were drawn from the submandibular vein following the 9^{th} treatment for a complete blood count.

An additional efficacy study was performed with SELP-targeted NPs. D4M.3A cells (1×10⁶ cells/mouse) were SC injected into 6-8 weeks old male C57BL/6 mice (Envigo C R S, Israel). Following 10 days (tumor size of about 50 mm³), the mice were randomized into 5 groups and were IV administered with SELP-targeted or non-targeted NPs or with the combination of free drugs, at 1 mg/kg DBF and 0.1 mg/kg TRM. When the first treated group reached tumor size above 1000 mm³, all treatments were discontinued. Body-weight and tumor size were monitored every other day and mice were euthanized when tumor volume above 1000 mm³ or when they lost more than 20% from their initial (day 0) body-weight. Blood samples were drawn from the submandibular vein following the 8^{th} treatment for biochemistry analysis.

Animal models in studies on BRCA-mutated cells: To evaluate the antitumor activity of drug-loaded NPs compared to free Drugs, 6-7 weeks old female BALB/c (Envigo C R S, Israel) mice were inoculated into the mammary fat pad with 0.2×10⁶ EMT6 cells. Tumor growth was monitored every two days with kaliper. Mice body weight was monitored also every two days. The tumors were allowed to establish for 15 days and then the mice were treated with talazoparib and SM56 as drug-loaded NPs (non-targeted and SELP-targeted) or free drugs and the corresponding controls, blank NPs (carrier only), saline and vehicle (saline containing 0.2% DMSO). Treatments were administered IV at 3.33 mg/kg SM56 and 0.33 mg/kg talazoparib, 3 time per week. The tumors were measured by a digital caliper according to the formula: width2×length×0.52. After the 7th treatment, the mice were euthanized when tumors reach 1000 mm³, in case of tumor ulceration or necrosis, or when mice display rapid weight loss (above 10% within a few days or 20% from the initial weight) or any sign of distress.

PD-L1 expression after treatments with talazoparib: EMT6 murine mammary carcinoma cells (obtained from ATCC) were incubated with talazoparib (0.1 nM, 10 nM, and 100 nM) for 24-72 hours and then harvested using FACS buffer (protein levels). 1×10⁶ cells were incubated with anti-PD-L1 at 1 μg/ml antibody concentration (Mouse PD-L1/B7-H1 Alexa Fluor® 647-conjugated Antibody; R & D Systems #FAB90781R) or isotype antibody (Rabbit IgG Alexa Fluor®647-conjugated Antibody, R & D Systems #IC1051R) for 1 hour on ice. The cells were washed twice with FACS buffer and the fluorescence intensity was measured by Attune NxT Flow Cytometer and analyzed by Kaluza software. All treatments were compared to the secondary antibody only or to the corresponding isotype control.

PD-L1i blocking activity: In order to assess the PD-L1i small molecule inhibition activity, murine EMT6 cells were seeded in 24-well plate (2×10⁴ cells/well). After 24 hours, the medium was replaced with medium containing PD-L1i (SM56; [Acúrcio et al. Journal for ImmunoTherapy of Cancer, 10(7), e004695 (2022)]) for 24-72 hours. The cells were then harvested with FACS buffer, washed and resuspended in FACS buffer. EMT6 cells (1×10⁶) were incubated with anti-PD-L1 at 1 μg/ml antibody concentration (Mouse PD-L1/B7-H1 Alexa Fluor®647-conjugated Antibody; R & D Systems #FAB90781R) or isotype antibody (Rabbit IgG Alexa Fluor®647-conjugated Antibody, R & D Systems #IC1051R) for 1 hour on ice. Additionally, to evaluate synergistic activity of PD-L1i with talazoparib, EMT6 cells were treated with the combination of PD-L1i and talazoparib for 48 hours. Later, same antibody and incubation conditions described previously were followed. These experiments were reproduced trice, and the data represent the mean±SD.

q-Real time PCR: Total RNA was isolated from cultured cells using the Direct-zol RNA Miniprep Plus (Zymo Research), according to the manufacturer's instructions. Once isolated, one microgram of RNA was reverse transcribed to cDNA using qScript cDNA synthesis kit (Quantabio, MA, USA). The cDNA was further diluted (1:50 in DNase/RNase-Free Water) for the qPCR. PD-L1 mRNA levels were assessed by SYBR green real-time PCR (StepOne plus; Thermo Fisher Scientific, Waltham, MA, USA) using PerFecTa SYBR Green FastMix ROX (Quanta BioSciences) and the following custom primers: murine PD-L1: forward, 5′-TTCAGATCACAGACGTCAAGCTG-3′, reverse, 5′-ATTCTCTGGTTGATTTTGCGGTA-3′. GAPDH: forward, 5′-ATTCCACCCATGGAATTC-3′, reverse, 5′-GGATCTCGCTCCTGGAAGATG-′3.

dSTORM imaging of PD-L1 after exposure of EMT6 cells to talazoparib: EMT6 cells were seeded in Ibidi μ-slide 8 well glass bottom chambered coverslips at a density of 5000 cells per well. After 24 hours incubation, cells were treated with Talazoparib for 48 hours (0, 10, 50 mM). After 48 hours cells were washed 1× with PBS and fixed using 3.7% formaldehyde solution for 10 minutes at room temperature. After fixation, cells were washed trice with PBS and incubated with 5% BSA solution overnight at 4° C. Antibody staining with anti-PD-L1 (Mouse PD-L1 Alexa Fluor 647 conjugated) or control antibody (Rabbit IgG Alexa Fluor 647 conjugated) was performed for 2 hours at room temperature at 1 μg/ml antibody dilution in PBS containing 5% bovine serum albumin (150 μl per well). Subsequently, cells were washed thrice with PBS and post-fixated using 1% formaldehyde solution for 10 minutes at room temperature. Finally, cells were washed thrice with PBS and stored at 4° C. before imaging. Before dSTORM imaging cells were incubated with 0.5 μg/ml WGA-488 for 10 minutes at room temperature to visualize the cell outline. Cells were washed thrice with PBS and incubated with STORM buffer (5% w/v glucose, 100 mM cysteamine, 0.5 mg/ml glucose oxidase, 40 μg/ml catalase in PBS). Before STORM imaging, a low resolution TIRF image was acquired at 5% 647 laser poser and 2% 488 laser power. For dSTORM imaging, cells were acquired for 20000 frames at 16 ms exposure time and 100% 647 laser power. Between 6 and 10 cells were imaged for each condition. dSTORM images were analyzed with the Nikon NIS elements software (version 5.21.01). dSTORM localizations were detected using Gaussian fitting, with a minimum intensity threshold height of 150 for the 647 nm channel. Molecules detected in 5 consecutive frames were considered as a single blinking event, while molecules detected in more than 5 frames were discarded. Drift correction was performed in the NIS elements software, based on an autocorrelation function. The resulting x-y coordinates of the detected localizations were imported and run through a custom MATLAB scrip to quantify the localization density per cell. Each cell was manually selected by drawing an ROI around the cell contour (detected in the 488 channel). Resulting localization densities per cell were plotted in a box plot using Origin 2020 software.

Analysis of mCherry-labeled EMT6 3D spheroids sprouting: 3D spheroid of EMT6 cells (2000 cells/spheroid) were prepared in DMEM medium supplemented with 0.24 w/v % methyl cellulose. The cells were deposited in 25 μL droplets into the inner side of a 20 mm dish and incubated for 48 hours at 37° C. when the plate is facing upside down to allow for spheroid formation. The spheroids were then embedded in Matrigel®, seeded in a 96-well plate, and monitored for cell invasion into the Matrigel®. The 3D spheroids were treated with talazoparib (1 nM, 100 nM and 1 μM) and their sprouting in the Matrigel® was evaluated by measuring the % red area with ImageJ software.

Murine splenocytes isolation and co-culture with EMT6 mCherry labeled 3D spheroids: Splenocytes were isolated from the spleen of adult BALB/c mice and cultured in RPMI medium supplemented with 10% FBS, 1% HEPES Buffer, 1% sodium pyruvate, 0.1% β-mercaptoethanol. The splenocytes were activated with 100 ng/mL of anti-CD3 (Ultra-LEAF™ Purified anti-mouse CD3 antibody; Biolegend #100340), 10 ng/mL of anti-CD28 Ultra-LEAF™ Purified anti-mouse CD28; Biolegend #102116) and 10 ng/mL E. coli-derived human IL-2 protein (R & D Systems #202-IL-500) for 48 hours. In parallel, EMT6 mCherry labelled spheroids were prepared by seeding the cells (500 cells/well/spheroid) in an ultra-low attachment round-bottom 96-well plate (Corning) and allowing them to form spheroids for 72 hours. Following, the medium was replaced with a fresh medium containing activated splenocytes (1:100 EMT6: splenocytes ratio) and talazoparib and/or SM56. The plate was then placed in the Incucyte Live cell analysis system (Essen Bioscience) and the treatment efficacy was assessed by measuring the mCherry fluorescent intensity from the spheroid for 72 hours.

Statistical methods: Data were expressed as mean±SD for in-vitro assays and as mean t standard error of the mean (SEM) for in vivo assays. Sample size (N) for each statistical analysis was added for each experiment. Statistical significance was determined using an analysis of variance (ANOVA) or t-test. P<0.05 was considered statistically significant. All statistical measurements were two-sided. Kaplan-Meier curve was created to assess the survival of mice in vivo. The software used for statistical analysis was GraphPad Prism 8.

### Example 1

### Design

In order to facilitate the penetration of the drugs into the brain, especially in the early stages of the micrometastases, the present inventors have envisioned to use a nano-sized poly(lactic-co-glycolic acid) (PLGA) drug delivery system for the encapsulation of DBF and TRM at a synergistic ratio. Co-encapsulation of the two drugs in a single nanoparticle (NP) was presumed to force the simultaneous delivery of the two drugs, at the required ratio for synergism, to each of the tumor cells specifically, and assure that the exposure to the two drugs would be the same in each of the tumor cells and enhance their therapeutic efficacy.

Traditionally, passive accumulation of NPs to the tumor site was mediated by the enhanced permeability and retention (EPR) effect [Matsumura, Y. & Maeda, H. Cancer Res 46, 6387-6392 (1986)], however, it has been shown to vary between tumor types and within the tumor itself [Monsky, W. L., et al. Cancer Res 59, 4129-4135 (1999); Rosenblum et al. Nat Commun 9, 1410 (2018)]. Both larger tumor volumes and micrometastases were shown to have variability in blood vessel permeability, and therefore the present inventors intended, inter alia, to actively target the NPs to these hard-to-reach regions, which are susceptible to subtherapeutic drug concentration and resistance development [Schroeder, A., et al. Treating metastatic cancer with nanotechnology. Nat Rev Cancer 12, 39-50 (2011); Bombelli et al. Lancet Oncol 15, e22-32 (2014); Prabhakar, U., et al. Cancer Res 73, 2412-2417 (2013); and Torchilin, V. P. AAPS J9, E128-147 (2007)].

For this reason, P-selectin (SELP) was targeted. Various cells (e.g., endothelial cells and tumor cells) were previously shown to express SELP, which was targeted by the present inventors as a feature be exploited to enhance the accumulation of NPs at both the tumor cells and the activated endothelial cells at the tumor site.

As SELP promotes metastasis by arresting circulating tumor cells at the pre-metastatic niche and enabling the tumor cells to extravasate through the activated blood vessels and facilitate colonization, the present inventors have intended to utilize the precursory expression of SELP, and by that to actively accumulate the targeted NPs at these restricted-penetration regions.

SELP-targeted polymeric nanocarriers containing multivalent sulfates as targeting agents were previously reported [Ferber, S., et al., supra; Shamay, Y., et al. supra; and Solhi, L., et al. supra] and the major driving forces for binding between SELP and sulfate/sulfonate groups present on the surface of nanocarriers are charge-charge interactions [Achazi, K., et al. Angew Chem Int Ed Engl 60, 3882-3904 (2021)] and counterion release after binding, as demonstrated in molecular dynamics (MD) simulations [Boreham, A., et al. Molecules 21, E22 (2015)]. But, to date, any synthesized amphiphilic polymers that contained sulfates had limited drug loading capacity due to the random distribution of hydrophobic and hydrophilic moieties. Moreover, as sulfate groups are negatively charged, the carriers have failed to encapsulate very hydrophobic drugs that lacked the availability of positively charged moieties. Furthermore, the hydrophilicity of the polymers can result in the fast release of their cargo already in the blood.

The present inventors have designed and successfully practiced PLGA-PEG-based NPs bearing SELP targeting moieties for targeting tumors, inter alia, at unreachable or sensitive regions, such as micrometastases.

### Example 2

### Evaluation of Combined Drugs Synergistic Activity

In order to select a synergistic ratio of DBF and TRM for their encapsulation within the NPs, a Combination index (CI) was calculated after exposing D4M.3A, 131/4-5B1, and A375 melanoma cells to different concentrations of the selected drugs. The data for Combination index (CI) of each cell line and treatment is summarized in Table 1 herein:

TABLE 1

| | CI values | | | CI values | | |
|---|---|---|---|---|---|---|
| | TRM 0.3 nM + DBF | | | DBF 1 nM + TRM | | |
| IC | D4M.3A | 131/4-5B1 | A375 | D4M.3A | 131/4-5B1 | A375 |
| 30 | 1.09 | 1.18 | 1.00 | 0.87 | 0.84 | 1.03 |
| 50 | 0.88 | 0.84 | 0.82 | 0.78 | 0.64 | 0.79 |
| 70 | 0.61 | 0.46 | 0.77 | 0.60 | 0.45 | 0.63 |

As can be seen from Table 1, based on the data presented in FIGS. 1A-F, the combined drugs showed synergism (CI<1) at the concentration that led to 50% and 70% inhibition of cell growth (IC50 and IC70, respectively) compared to each drug separately, and a 1:10 ratio of TRM:DBF was chosen for further experiments.

### Example 3

### Design and Characterization of Drug-Loaded NPs

The polymer concentration in the organic phase is a critical variable that affects the physicochemical properties of the NPs [Lim, J. M., et al. Nanomedicine 10, 401-409 (2014); and Rhee, M., et al. Adv Mater 23, H79-83 (2011)]. Therefore, two different concentrations of PLGA (10 and 6 mg/mL) were evaluated for the formulation of the NPs.

As PEG provides a hydrophilic shielding to the NPs and increases the blood circulation time while reducing their liver uptake [Beletsi, A. et al. Int J Pharm 298, 233-241 (2005); and Rafiei, P. & Haddadi, A. Int J Nanomedicine 12, 935-947 (2017)], different ratios of PLGA: PLGA-PEG (2:1 or 4:1, respectively) were also examined.

Physicochemical characterization (Drug loading content (DLC), drug loading efficiency (DLE), conductivity, and dispersity) of the different NPs are presented in FIGS. 2A-C and are summarized in Table 2 below.

TABLE 2

| | | Size | | | | | | |
|---|---|---|---|---|---|---|---|---|
| Polymer | PLGA:PLGA- | by | DLC | DLC | DLE | DLE | Z | |
| concentration | PEG | DLS | DBF | TRM | DBF | TRM | potential | Dispersity |
| [mg/mL] | ratio | [nm] | [wt %] | [wt %] | [%] | [%] | [mV] | index [Ð] |
| 6 | 2:1 | 88 ± 3 | 6.7 ± 0.5 | 0.82 ± 0.06 | 33 ± 2 | 36 ± 2 | −0.9 ± 0.7 | 0.17 ± 0.02 |
| | 4:1 | 92 ± 3 | 5.6 ± 0.8 | 0.51 ± 0.09 | 25 ± 3 | 20 ± 2 | −0.6 ± 0.7 | 0.17 ± 0.04 |
| 10 | 2:1 | 92 ± 9 | 3.1 ± 0.5 | 0.27 ± 0.05 | 25 ± 3 | 19 ± 3 | −0.4 ± 0.6 | 0.19 ± 0.01 |
| | 4:1 | 107 ± 1 | 5.1 ± 0.1 | 0.54 ± 0.03 | 38 ± 1 | 37 ± 2 | −1.4 ± 0.5 | 0.15 ± 0.03 |

Table 2 shows that the formulation that contained a ratio of 2:1 PLGA: PLGA-PEG at 6 mg/mL polymer concentration produced smaller NPs with higher drug loading content compared to the other formulations. As can be seen in FIGS. 2D and 2E, 50% release time for DBF was 10-12 hours and TRM was 16-17 hours, for all formulations, which indicates that the release profile was comparable between all four formulations and did not present a higher release rate due to the higher drug content.

Based on these results, a formulation comprising a ratio of 2:1 PLGA: PLGA-PEG at 6 mg/mL polymer concentration was used. Blank (empty) NPs and single drug-loaded NPs were prepared and used as controls.

Physicochemical characterization (Drug loading content (DLC), drug loading efficiency (DLE), conductivity, and dispersity) of the NPs are presented in FIGS. 3A-C and are summarized in Table 3 below.

TABLE 3

| | Size by | Size by | DLC | DLC | DLE | DLE | | |
|---|---|---|---|---|---|---|---|---|
| | DLS | TEM | DBF | TRM | DBF | TRM | Z potential | Polydispersity |
| Sample | [nm] | [nm] | [wt %] | [wt %] | [%] | [%] | [mV] | (PD) index [Ð] |
| Blank NPs | 84 ± 12 | 69 ± 6 | — | — | — | — | −0.35 ± 0.35 | 0.16 ± 0.06 |
| DBF + | 88 ± 3 | 74 ± 12 | 6.74 | 0.82 | 33 | 36 | −0.94 ± 0.67 | 0.17 ± 0.02 |
| TRM NPs | | | | | | | | |
| TRM NPs | 81 ± 6 | 75 ± 16 | — | 0.76 | — | 27 | −0.12 ± 0.09 | 0.21 ± 0.02 |
| DBF NPs | 85 ± 4 | 70 ± 10 | 6.77 | — | 32 | — | −0.83 ± 1.26 | 0.17 ± 0.04 |

Table 3 and FIGS. 3A-D show that the NPs average size was about 80 nm for all formulations, and the average dispersity index indicated a narrow and homogenous size distribution (Ð=0.18). DLE was similar between the single drug-loaded and the dual drug-loaded NPs (30%), and the average DLC for DBF was 6.7 wt % and for TRM 0.80 wt %. The NPs size and dispersity index were constant during the 72 hours incubation in PBS, and the zeta potential remained almost neutral. The release profile of the dual-loaded NPs was similar to the single-loaded NPs, in which 50% drug release was recorded for DBF after 10 hours and TRM 12 hours.

TEM analyses were also performed and as seen in FIGS. 4A-E, the images showed a spherical and homogeneous NPs structure with an average diameter of approximately 70 nm for all NPs formulations.

To further assess the stability and release profile of the NPs under physiological conditions, the dual-loaded DBF+TRM NPs were incubated in a growth medium containing fetal bovine serum (FBS). As the results on FIG. 5A depict, the size of the NPs was constant throughout the duration of the experiment (average size 95±12 nm), and TRM release profile (see, FIG. 5B) was preserved with 50% drug release after 14 hours. DBF release profile was faster, with 50% drug release after 5 hours.

Altogether, these findings suggest that the drug encapsulation did not change the NPs structure or size and corresponded to the DLS measurements.

### Example 4

### The Effect of a Combined Treatment on Proliferation of Melanoma Cells

The cytotoxic effect of TRM, DBF, and the combined DBF+TRM drug-loaded NPs was evaluated on D4M.3A and A375 melanoma cells' viability.

Based on the data presented in FIGS. 6A and 6B, IC₅₀ values were calculated by GraphPad Prism using nonlinear regression analysis for [Inhibitor] vs. normalized response. The IC₅₀ values of the different treatments on D4M.3A and A375 cell lines are summarized in Table 4 below.

TABLE 4

| | IC₅₀ values (nm) | | | | | | |
|---|---|---|---|---|---|---|---|
| Treatment/ | DBF | TRM | DBF + TRM | DBF | TRM | DBF + TRM | DBF NPs + |
| Cell line | free | free | free | NPs | NPs | NPs | TRM NPs |
| D4M.3A | 4.9 | 8.6 | 3.0 | 3.9 | 5.2 | 3.3 | 1.9 |
| A375 | 13.6 | 8.5 | 9.7 | 2.1 | 3.1 | 1.3 | 6.3 |

As shown in Table 4, while the IC₅₀ values of the combined DBF+TRM treatment were lower than each of the two drugs given as monotherapy, there was no difference between the IC₅₀ values of the combined free drugs and the dual-loaded NPs. Without being bound to any particular theory, it is assumed to occur since most of the drugs are being released during the 72 hours incubation period.

Next, the cytotoxic effect was evaluated on melanoma 3D spheroids. The spheroids were first treated with three different dilutions of free TRM, DBF, and their combination at 1:10 ratio, to determine the concentration that will produce a synergistic effect.

As can be seen from the results in FIGS. 7A-C and FIGS. 8A-C, the ability of the spheroids to invade the Matrigel® was significantly decreased when DBF and TRM were combined at the synergistic concentrations of 10 nM DBF and 1 nM TRM compared to 10 nM DBF or 1 nM TRM monotherapies, both in D4M.3A and WM115 melanoma-treated cells. These results indicate that the synergistic effect of the free drugs combination of spheroids sprouting is dose-dependent.

FIG. 9 shows that the selected synergist concentrations of 10 nM DBF- and 1 nM TRM-loaded NPs reduced the invasion ability of WM115 melanoma spheroids to a higher extent compared to the mono-treatments and to the combination of DBF and TRM free drugs.

As melanoma frequently develops secondary lesions in the brain, with an incidence of BRAF and NRAS mutations even higher than those observed in primary melanoma tumor, a more complex 3D model of spheroids was developed: the melanoma cells were combined with brain resident cells (astrocytes, microglia, and microvascular brain endothelial cells—hCMEC) to create a 3D MCTS model of melanoma brain metastasis (MBM).

The 3D MBM MCTS of WM115 mCherry-labeled cells were treated with free drugs or drug-loaded NPs, using the synergist concentrations of 10 nM DBF and/or 1 nM TRM.

As can be seen in FIGS. 10A-C, a significant reduction in melanoma invasion was observed, even though astrocytes and other cells of the brain microenvironment were previously shown to shield and protect melanoma cells from chemotherapy and cooperate to sustain the growth of secondary lesions [Doron, H., et al. Cell Rep 28, 1785-1798.e1786 (2019)]. Furthermore, the treatments with TRM and/or DBF did not affect the invasion ability of the brain resistant cells, and the blank NPs did not show cell toxicity.

Next, the inhibitory effect of the combined treatment was assessed on a murine melanoma spheroid model. To calibrate the concentration needed to demonstrate a synergistic effect, mCherry labeled D4M.3A spheroids were treated with different dilutions of free DBF, TRM, and their combination at 1:10 (TRM:DBF) ratio. FIGS. 8A-C show that the efficacy of the combined drugs compared to individual treatment was not as successful as in the WM115 spheroids. Without being bound to any particular theory, it may be explained by the high growth rate and sprouting abilities of D4M.3A spheroids.

Nevertheless, as seen in FIGS. 11A and 11B, the spheroids were treated with the NPs and free drugs, similar to the WM115 spheroids, and reduced the sprouting of the spheroids to a higher extent than the blank (empty) NPs and the untreated spheroids.

Overall, exemplary DBF- and TRM-loaded NPs successfully inhibited the proliferation of melanoma cell lines and reduced spheroid sprouting.

### Example 5

### The Accumulation of NPs in Tumor In Vivo

Before examining the effect of PLGA NPs on mice, hemocompatibility was assessed.

For the examination of hemocompatibility of the NPs, an erythrocytes suspension was incubated with the NPs for 1 hour.

As can be seen in FIG. 12A, the highest NPs concentration tested (2 mg/mL) caused minimal hemolysis, similarly to the results obtained for the negative control (dextran 70 kDa). Therefore, NPs at a concentration of 2 mg/mL were used for IV treatments.

The motor coordination, imbalance, memory, and learning abilities of mice treated with free drugs or drug-loaded NPs were evaluated by a RotaRod test that was taken before the 1^{st} treatment and after the 7^{th} treatment.

As FIG. 12B indicates, the latency to fall was longer after the 7^{th} treatment in all groups. These data suggest that the neuromuscular coordination of mice was not affected by the administration of the PLGA NPs.

Next, the tumor accumulation of PLGA Cy5-labeled NPs was compared to free DBF conjugated to Cy5, as described in Pisarevsky et al. (supra). Free TRM lacked binding sites to Cy5 and therefore was not used as an additional control. C57BL/6 male mice were SC inoculated with 1·10⁶ D4M.3A cells and were allowed to establish tumors. Once the tumor size reached about 230 mm³, the mice were IV-treated with PLGA-Cy5 NPs or DBF-Cy5 and the Cy5 signal was monitored for 24 hours. The results are presented in FIGS. 13A-E.

As can be seen in FIG. 13D, 3 hours post the injection, the accumulation of PLGA-Cy5 NPs into the tumor was 6.8 times higher than the free DBF-Cy5, with 1.18 scaled counts/s*mm² and 0.17 scaled counts/s*mm², respectively. 24 hours post the injection, as FIG. 13E depicts, the total tumor accumulation of PLGA-Cy5 NPs (represented by the AUC) was twice higher than the free DBF-Cy5 (7.115 and 3.786, respectively). In effect, according to the release profile (FIG. 2E), most of the drug was still entrapped inside the NPs at T=3 hours (70% for DBF and 81% for TRM), and therefore these NPs could increase the drug concentration at the tumor site due to their enhanced accumulation by the EPR effect.

In addition, after 24 hours, the free drug accumulated in the heart to a higher extent than the NPs, causing cardiac adverse events, which were previously shown to be related to DBF and TRM combined treatments (the inset in FIG. 13E) [Robert, C., et al. N Engl J Med 381, 626-636 (2019); Lamore, et al. Chem Res Toxicol 33, 125-136 (2020); and Bronte, E., et al. Pharmacol Ther 192, 65-73 (2018)].

These results indicate that the PLGA NPs accumulate at the tumor site within 3 hours following their systemic administration.

### Example 6

### In Vivo Anti-Tumor Activity of Drug-Loaded NPs

After the biocompatibility and enhanced tumor accumulation of the NPs was observed, the in vivo anti-tumor activity of drug-loaded NPs was evaluated.

C57BL/6 Mice were SC inoculated with D4M.3A cells and were allowed to establish tumors for 10 days (tumor size of about 180 mm³). Then, mice were treated with the combination of free DBF and TRM or with DBF- and TRM-loaded NPs at 3 mg/kg DBF and 0.3 mg/kg TRM, QOD. The mice were treated 7 times before the first group (PBS) reached the endpoint of tumor size above 1000 mm³, as illustrated in FIG. 14A.

As seen from the resulting tumor size in FIG. 14B, the combined therapy demonstrated increased anti-tumor activity compared to the controls without leading to substantial change in bodyweight in safety evaluations (FIG. 14C). As seen in FIGS. 15A-D, the expression of phosphorylated kinases, which reflects the level of inhibition of BRAF and MEK by DBF and TRM, was inhibited by 90-fold for pBRAF and by 80-fold for pMEK and pERK after the seventh treatment compared to PBS-treated mice.

These data indicate that treatments with DBF- and TRM-loaded PLGA NPs can inhibit the MAPK pathway in vivo.

In order to exploit the synergistic effect which led to an increased anti-tumor effect under reduced dosing, a dose reduction experiment was conducted.

C57BL/6 mice were inoculated subcutaneously with D4M.3A cells and were allowed to establish tumors. At day 10, when the average tumor volume reached 70 mm³, the mice were randomized into groups of 4-7 mice each and treated systemically with the combination of DBF (2 or 1 mg/kg, QOD) and TRM (0.2 or 0.1 mg/kg, QOD) as drug-loaded NPs or free drugs, respectively. A schematic presentation of the study protocol and timeline is presented in FIG. 16A.

The study results are presented in FIGS. 16B-G. The two drug-loaded NPs demonstrated superior efficacy by retaining a small tumor volume (below 250 mm³) for 57 (high dose) and 50 (low dose) days, compared to the corresponding doses of free drugs, which maintained this volume only until days 27 and 23, respectively, as depicted in FIGS. 16B-E. Additionally, the tumor growth was suppressed for a longer period, which suggests that the exposure of the drugs to the tumor cells was increased by the NPs. A significantly prolonged survival of the mice treated with the NPs compared to free drugs is also observed.

FIG. 16F shows that the median survival rate was 71 days for both doses of the NPs whereas the free drugs' median survival rate was 39 days for the low dose and 52 days for the high dose.

Toxicity evaluations were also performed to determine any induced adverse events that could arise from the systemic administration of the treatments. No group suffered from weight loss as seen in FIG. 16G. Also, a complete blood count was conducted after the 9^{th} treatment and did not show abnormalities (below 25% or above 75% percentiles) for the drug-loaded NPs, free drugs, and vehicle groups (data not shown).

Overall, combined DBF- and TRM-loaded NPs reduced tumor growth, and prolonged survival compared to free drugs, while not causing abnormalities in blood counts.

### Example 7

### Expression of SELP in Tumor Tissues

To further improve the therapeutic efficacy, the surface of the exemplary drug-loaded NPs was modified with SELP-binding moieties to actively target the tumor site by the NPs. For this purpose, the expression of SELP was assessed in murine and human melanoma tissues in comparison with healthy tissues.

The immunostaining for SELP on D4M.3A murine samples, presented in FIG. 17A-C, show higher expression of SELP in primary tumor and brain metastases tissues compared with healthy skin and brain tissues.

To assess the clinical relevancy of SELP-targeting, three patient-derived human samples were examined.

The results are presented in FIG. 17D-E. The samples showed elevated expression of SELP in primary tumors and in MBM compared with healthy brain samples. Furthermore, SELP expression was even higher in MBM specimens than in the primary tumor sample, which is in accordance with previous data.

Next, before examining the binding abilities of potential targeting moieties, a reliable in vitro model was needed. Since D4M.3A and WM115 cells grown in 2D cultures demonstrated low expression levels of SELP, 3D in vitro spheroids models were tested as they recapitulate better the tumor physiological conditions found in vivo [Pozzi, S., et al. Adv Drug Deliv Rev 175, 113760 (2021)].

Indeed, SELP expressions, presented in FIGS. 17F and 17G, was two times higher in cells grown in 3D spheroids than in 2D cultures. Additionally, when D4M.3A cells were combined with endothelial cells (bEnd.3) to form multicellular spheroids, SELP staining was three times higher compared to each cell line grown separately in 2D culture, as seen in FIGS. 17H-I.

These results indicate that SELP is highly expressed in tumor tissues and 3D spheroids tumor models.

### Example 8

### Design and Chemical Syntheses of SELP-Targeted NPs

After validating the expression of P-selectin, two modified PLGA-PEG polymers were synthesized and evaluated for P-selectin binding.

PEG polymer was incorporated into the polymeric matrix of the non-targeted NPs to benefit the hydrophilic stealth of PEG, which reduces NPs aggregation and provide longer blood circulation times. Therefore, the synthetic approach was to introduce the sulfate groups at the terminal end of the 2 kDa PEG in PLGA-PEG polymer, so that upon formation of NPs, sulfate groups would be multivalently presented on the surface of the NPs.

It was previously shown that the degree of sulfation in a hyperbranched polyglycerol polymer controls the binding affinity of the carrier to the L/P-selectin. Therefore, two polymers (PLGA-PEG-SO₃ and PLGA-PEG-Glycerol-(SO₃)₂, FIGS. 18A and 19A were synthesized, containing one and two sulfate groups, respectively.

The exemplary sulfated polymer, PLGA-PEG-SO₃ was synthesized as described in FIG. 18A, and as follows.

### 1. Preparation of Polymer 1

Synthesis of homobifunctional PEG-OMs: (based on Dey et al. Mimicking of Chondrocyte Microenvironment Using In Situ Forming Dendritic Polyglycerol Sulfate-Based Synthetic Polyanionic Hydrogels. Macromol Biosci 16, 580-590 (2016)): PEG-OH with a molecular weight of 2000 gram/mol (16.0 grams, 16 mmol of OH groups) was melted at 65° C. under high vacuum to remove traces of water overnight. After cooling to room temperature (RT) the PEG-OH was dissolved in 150 mL anhydrous dichloromethane (DCM) under argon. Dry triethylamine (11.0 mL, 80 mmol, 5 equivalents) was added while stirring and the mixture was cooled with an ice bath, followed by the dropwise addition of methanesulfonyl chloride (4.9 mL, 64 mmol, 4 equivalents). The reaction proceeds overnight under argon at room temperature (RT). The crude mixture was diluted with DCM (200 mL) and washed twice with brine (200 mL). The organic layer was dried with sodium sulfate and concentrated under vacuum. The residue was precipitated in diethyl ether and stirred vigorously for 2 hours, filtered, and washed again with diethyl ether to remove traces of excess amine and methanesulfonyl chloride. Drying under high vacuum yielded the desired above product (97% yield) as a white powder.

¹H NMR (400 MHz; DMSO-d₆; 25° C.): δ (ppm)=4.3 (m, 4H, SO₂OCH₂—PEG), 3.7-3.32 (m, 180H, PEG-backbone), 3.18 (s, 6H, CH₃SO₂).

Synthesis of homobifunctional PEG-NH₂ (Polymer 1): Homobifunctional PEG-OMs (15.0 grams) was added to 150 mL 25% aqueous ammonia solution and stirred for 4 days at room temperature (RT) in a sealed flask. The ammonia was evaporated under high vacuum, the pH of the aqueous solution was raised to 13 with 1N NaOH and the solution was extracted three times with 100 mL dichloromethane. The organic layers were combined and concentrated. The product was precipitated in diethyl ether and stirred vigorously for 2 hours, filtered and washed again with diethyl ether. Drying under high vacuum yielded the desired product (PEG-NH₂) as a white powder (92% yield).

¹H NMR (400 MHz; DMSO-D6; 25° C.): δ (ppm)=3.68 (m, 4H, —OCH₂—CH₂), 3.75-3.62 (m, 180H, PEG backbone), 2.65 (t, 4H, —CH₂—CH₂—NH₂).

2. Synthesis of NH₂-PEG-SO₃ (Polymer 2): Homobifunctional PEG-NH₂ (Polymer 1) (1.0 gram, 0.5 mmol, 1 equivalent) was taken in a round bottom flask equipped with a magnetic stir bar and dissolved in tetrahydrofuran (THF) (10% w/v). Then, 10% w/v propane sulfone (61 mg, 0.5 mmol, 1 equivalent) in THF was added dropwise to the PEG solution and the reaction was stirred at 50° C. for 5 hours. Then, as the reaction proceeded, the product was precipitated, filtered, washed with cold THF, and dried under high vacuum overnight.

¹H NMR (400 MHz; CDCl₃; 25° C.): δ (ppm)=4.0-3.5 (m, 180H, PEG backbone), 3.05 (t, 2H, CH₂—CH₂—CH₂—SO₃H), 2.81 (t, 4H, —CH₂—CH₂—NH₂), 2.2 (m, 2H, —CH₂—CH₂—CH₂—SO₃H).

3. Synthesis of PLGA-PEG-SO₃ (Polymer 3): PLGA (Resomer® RG 502H, Sigma) (1.0 gram, 0.143 mmol, 1 equivalent) was taken in a round-bottom flask, then N-Hydroxysuccinimide (NHS) (0.083 gram, 0.715 mmol, 5.0 equivalents) and dry DCM (10 mL) were added and stirred at RT. Next 1-Ethyl-3-(3-dimethylaminopropyl)carbodiimide (EDC) (0.11 gram, 0.572 mmol, 4 equivalents) was added and the reaction was stirred overnight. Following, DCM was evaporated, and the product was precipitated in cold methanol (MeOH) thrice. Finally, the residue was collected and dried under high vacuum to obtain PLGA-NHS. (90% yield)

¹H NMR (400 MHz; CDCl₃; 25° C.): δ (ppm)=5.5-5.1 (m, 92H, —O—CH(CH₃)—CO—), 5.0-4.0 (m, 184H, —O—CH₂—CO—), 2.9-2.75 (m, 4H, CH₂ from NHS), 1.7-1.5 (m, 276H, —O—CH(CH₃)—CO—).

Polymer 2 (0.27 gram, 0.129 mmol, 3 equivalents) was placed in a round bottom flask. N,N-Diisopropylethylamine (DIPEA) (0.04 mL, 0.216 mmol, 5.0 equivalents) and dry chloroform (10 mL) were added and stirred at RT for 10 minutes followed by the addition of PLGA-NHS (0.3 gram, 0.043 mmol) and stirred at RT overnight. Then the reaction mixture was heated at 45° C. for 6 hours and the chloroform was removed. The residue was washed with cold MeOH thrice, and the residue was collected. The final product was dried under high vacuum to obtain PLGA-PEG-SO₃ as a white solid (70% yield).

¹H NMR (400 MHz; CDCl₃; 25° C.): δ (ppm)=5.5-5.1 (m, 92H, —O—CH(CH₃)—CO—), 5.0-4.0 (m, 184H, —O—CH₂—CO—), 4.0-3.5 (m, 180H, PEG CH₂), 1.7-1.5 (m, 276H, CH₃).

The structure of PLGA-PEG-SO₃ was confirm by ¹H NMR. The introduction of peaks at 2.79 ppm confirms the formation of amino functionalization from hydroxy groups (FIG. 18B, red spectrum). CH₂ peak of 1,3-propane sulfone was observed at 2.2 ppm further confirms the functionalization. Based on the ¹H NMR ratio between PEG and 1,3-propane sulfone, the introduction of a single group was established.

Functionalization with two sulfonate groups was expected to result in a non-reactive PEG towards the next reaction (FIG. 18B, green spectrum). Therefore, the introduction of PEG to PLGA was verified by comparing the ratio between the PEG (peak at 4-3.5 ppm) and PLGA Lactic acid's CH (peak at 5.5-5.1 ppm). Excess of PEG was removed by washing with MeOH and dialysis (FIG. 18B, blue spectrum).

Next, two sulfates were introduced at the terminal end of PEG by coupling one glycerol unit for the preparation of PLGA-PEG-Glycerol-(SO₃)₂.

Preparation of PLGA-PEG-Glycerol-(SO₃)₂: Solketal was activated using CDI, and then it was reacted with PEG diamine to obtain polymer 4 (FIG. 19A). Here the coupling of one unit of glycerol was confirmed by ¹H NMR and HRMS. First, the formation of 3 was confirmed by ¹H NMR (FIG. 19B, red spectrum), and after coupling to PEG diamine, corresponding peaks of sol ketal was observed between 4.5 and 4.0 ppm along with the peaks of NH at 5.94, methyl groups from acetals at 1.5-1.3 ppm and PEG peaks 4.5-4.0 ppm (FIG. 19B, blue spectrum).

### 1. Preparation of Polymer 5

Synthesis of compound 3: Solketal (0.5 gram, 3.78 mmol, 1 equivalent) was placed in a flame dried round bottom flask and dry DCM (8 mL) was added under argon. Then 1,1′-carbonyldiimidazole (CDI) (0.79 gram, 4.9 mmol, 1.3 equivalents) was dissolved in dry DCM (3 mL) and added to the above reaction mixture instantly. The reaction mixture was stirred at RT for 3 hours and washed with brine. The organic layer was dried with sodium sulfate, and a flash column was performed using ethyl acetate/hexane. Pure product (white oil) was obtained with 20% ethyl acetate/hexane (70% yield).

¹H NMR (400 MHz; CDCl₃; 25° C.): δ (ppm)=8.15 (s, 1H, —N—CH═N—CH), 7.44 (s, 1H, —N(CH)—CH═CH—), 7.07 (s, 1H, —N(CH)—CH═CH—), 4.55-4.35 (m, 3H, —O—CH₂—CH—), 4.2-4.1 (m, 1H, —O—CH₂—CH—CH₂), −3.9-3.8 (m, 1H, —O—CH₂—CH—CH₂), 1.5-1.3 (s, 6H, —CH₃).

Synthesis of polymer 4: Homobifunctional PEG-NH₂ (Polymer 1) (1.0 gram, 0.5 mmol, 1 equivalent) was placed in a round bottom flask equipped with a magnetic stir bar. Then 18 mL dry chloroform and DIPEA (0.13 mL, 0.75 mmol, 1.5 equivalents) were added under argon and the reaction mixture was stirred for 5 minutes at RT. Then compound 3 (0.178 gram, 0.6 mmol, 1.2 equivalents) dissolved in dry chloroform (2 mL) was added dropwise to the reaction mixture and stirred overnight at RT. Then chloroform was concentrated, and the product was precipitated in cold diethyl ether. Following, the white precipitate was collected and dried under high vacuum to obtain polymer 4 as a white solid (76% yield).

¹H NMR (400 MHz; CDCl₃; 25° C.): δ (ppm)=5.44 (s, 1H, NH), 4.3-4.1 (m, 5H, CH₂ and CH from sol ketal), 4.0-3.25 (m, 180H, PEG backbone), 2.94 (t, 2H, —CH₂—CH₂—NH—CO—O—) and 2.8 (t, 2H, —CH₂—CH₂—NH₂), 1.5-1.3 (m, 6H, CH₃).

Synthesis of polymer 5: Polymer 4 was dissolved in 4 mL of dry DCM and 3 mL TFA was placed in 0.5 mL of dry DCM and added dropwise to the polymer at 0° C. and stirred overnight. TFA and DCM were evaporated, and the product was dissolved in DCM followed by precipitation in cold diethyl ether. The residue was collected and dried under high vacuum (Yield 90%).

¹H NMR (400 MHz; CDCl₃; 25° C.): δ (ppm)=5.94 (s, 1H, NH), 4.2-4.0 (m, 2H, —NH—CO—O—CH₂—CH(OH)—CH₂—OH), 4.0-3.5 (m, 180H, PEG backbone), 3.5-3.25 (m, 3H, CH—OH and CH₂—OH from glycerol), 3.25 (t, 2H, 2H, —CH₂—CH₂—NH—), 2.8 (t, 2H, —CH₂—CH₂—NH₂).

ESI-MS spectra of PEG diamine and polymer 5 showed Mp (peak with high intensity) at 1823.1 gram/mol and 1940.9 gram/mol, respectively. The m/z difference of 118 gram/mol corresponds to one glycerol unit. Further, all the masses in the distribution can be explained by the following equations:

Equation i. M (PEG-diamine)=(n−1)*44.026 (CH₂CH₂O)+44.077 (CH₂CH₂ NH₂)+16.023 (NH₂)+1.008 (H⁺) Equation ii. M (PEG-Glycerol-OH, polymer 5)=(n−1)*44.026 (CH₂CH₂O)+162.165 (CH₂CH₂ NH—COO—CH₂CH(OH)CH₂OH)+16.023 (NH₂)+1.008 (H⁺)

2. Preparation of polymer 7: Deprotection of acetal groups leads to peak disappearance at 1.7-1.5 ppm (FIG. 19C, red spectrum). After coupling of solketal group, single mass distribution was obtained, and all the corresponding peaks were identified using Equation ii (data not shown).

Then, polymer 5 was reacted with NHS activated PLGA to obtain polymer 6, which was characterized by ¹H NMR spectra (FIG. 19C, green spectrum).

Moreover, by integrating the peaks of CH protons of PLGA chains and corresponding CH₂ protons from PEG, the ratio can be calculated, to confirm the coupling of a single PEG chain to one end of PLGA.

2.1 Synthesis of polymer 6: PLGA (Resomer® RG 502H, Sigma) (0.5 gram, 0.072 mmol, 1 equivalent), was placed in a round bottom flask equipped with a magnetic stir bar. Dry DCM (8 mL) and triethylamine (0.06 mL, 0.432 mmol, 6 equivalents) were added and stirred at RT for 10 minutes. Then acetyl chloride (0.03 mL, 0.486 mmol, 4.5 equivalents) was added at 0° C. and stirred at RT overnight. Following, DCM was removed, and the product was precipitated in cold MeOH thrice. The residue was collected and dried under high vacuum to obtain polymer 6 as a white solid (93% yield).

¹H NMR (400 MHz; CDCl₃; 25° C.): δ (ppm)=5.5-5.1 (m, 92H, CH), 5.0-4.0 (m, 184H, CH₂), 2.18-2.10 (s, 3H, CH₃), 1.7-1.3 (m, 276H, CH₃).

2.2 Synthesis of PLGA-PEG-Glycerol-OH (polymer 7): Polymer 6 (1.0 gram, 0.143 mmol, 1 equivalent) was placed in a round bottom flask. Then NHS (0.083 gram, 0.715 mmol, 5.0 equivalents) and dry DCM (10 mL) were added and stirred at RT. Then EDC (0.11 gram, 0.572 mmol, 4 equivalents) was added and stirred overnight. DCM was removed and the product was precipitated in cold MeOH thrice. The residue was collected and dried under high vacuum to obtain NHS ester of polymer 6 as a white solid (90% yield).

¹H NMR (400 MHz; CDCl₃; 25° C.): δ (ppm)=5.5-5.1 (m, 92H, CH), 5.0-4.0 (m, 184H, CH₂), 2.9-2.75 (m, 4H, NHS), 2.18-2.10 (s, 3H, CH₃), 1.7-1.5 (m, 276H, CH₃).

Polymer 5 (0.27 gram, 0.129 mmol, 3 equivalents) was taken in a round bottom flask. Then DIPEA (0.04 mL, 0.216 mmol, 5.0 equivalents) and dry chloroform (10 mL) were added and stirred at RT for 10 minutes. Then, the NHS ester of polymer 6 (0.3 gram, 0.043 mmol, 1 equivalent) was added and stirred at RT overnight. Next, the reaction mixture was heated at 45° C. for 6 hours, and chloroform was removed. The residue was washed with cold MeOH thrice and collected. Then it was dried under high vacuum to obtain polymer 7 as a white solid (79% yield).

¹H NMR (400 MHz; CDCl₃; 25° C.): δ (ppm)=5.5-5.1 (m, CH, 92H), 5.0-4.0 (m, CH₂, 184 H), 4.0-3.5 (m, PEG CH₂, 180H), 2.18-2.10 (s, 3H, CH₃), 1.7-1.5 (m, 276H).

### 3. Preparation of PLGA-PEG-Glycerol-(SO₃)₂

Sulfation using SO₃-DMF complex leads to the formation of PLGA-PEG-Glycerol-(SO₃)₂, and the presence of both ¹H protons from PLGA and PEG confirmed the synthesis of the target polymer. In addition, the ratio of PLGA and PEG chains was in the desired range.

Sulfation was confirmed by checking the FTIR of both sulfated PLGA-PEG-Glycerol-(SO₃)₂ (polymer 8) and PLGA-PEG-Glycerol-OH (polymer 7) The appearance of two FTIR bands at 1422 and 1385 cm-1 compared to polymer 7 confirmed the sulfation (data not shown).

Sulfation was performed following coupling of polymer 5 to PLGA, which also contain a hydroxy group on another side. ¹H NMR confirmed the presence of methyl ester. As expected, the peak was observed at 2.2-2.1 ppm in both polymers PLGA-PEG-Glycerol-OH (7) and PLGA-PEG-Glycerol-(SO₃)₂. Originally the corresponding peaks could be seen in the ¹H NMR of 6 as well (FIG. 19C).

Synthesis of PLGA-PEG-Glycerol-(SO₃)₂ (polymer 8): Polymer 7 (0.236 gram, Mw 9000, 0.052 mmol, 1 equivalent) was dissolved in 5 mL dry dimethylformamide (DMF) and heated to 65° C. Then, SO₃-DMF complex (80 mg, 0.524 mmol, 10 equivalents) was dissolved in 3 mL dry DMF and added dropwise to the solution of 7 under argon. Then the reaction mixture was heated to 65° C. for 24 hours and stirred at RT for another 24 hours. Then DMF was removed using high vacuum and aqueous NaHCO₃ solution was added and stirred for a few minutes. The product was dialyzed in Milli-Q® water keeping NaCl solution outside for 24 hours and then Milli-Q® water respectively for another 48 hours. The solution present inside the dialysis was lyophilized to obtain PLGA-PEG-Glycerol-(SO₃)₂ as a white solid (72% yield).

¹H NMR (400 MHz; CDCl₃; 25° C.): δ (ppm)=5.5-5.1 (m, 92H, CH), 5.0-4.0 (m, 184H, CH₂), 4.0-3.5 (m, 180H, PEG backbone), 2.18-2.10 (s, 3H, CH₃), 1.7-1.5 (m, 276H, CH₃).

An additional exemplary sulfated polymer, PLGA-PEG-Glycerol-(SO₃)₂ amphiphilic hybrid, was synthesized as described in FIG. 28A, using Click chemistry, and as follows.

HO-PLGA₁₂ₖ-PEG₂ₖ-allyl (polymer 11) was obtained from Creative PEGWorks. It was characterized as follows: ¹H NMR (400 MHz, Chloroform-d) δ 5.97-5.84 (m, 1H, CH₂═CH—CH₂—O—), 5.33-5.06 (m, 83H, —O—CH(CH₃)—CO—, PLGA backbone), 4.95-4.55 (m, 100H, —O—CH₂—CO—, PLGA backbone), 4.09-3.92 (m, 2H, CH₂═CH—CH₂—O—), 3.85-3.43 (m, 181H, —O—CH₂—CH₂—O—, PEG backbone), 1.78-1.38 (m, 250H, —O—CH(CH₃)—CO—, PLGA backbone). SEC (DMF+25 mM NH₄Ac, PEG standards calibration): Mn=3.8 kDa, Ð=2.2; see, FIG. 28B.

Synthesis of AcO-PLGA₁₂ₖ-PEG₂ₖ-allyl (polymer 12): Polymer 11 (1.95 gram, Mw 11 kDa, 1 equivalents) was dissolved in DCM (10 mL per 1 gram of polymer). Et₃N (240 μL, 10 equivalents) and Acetyl-Cl (250 μL, 20 equivalents) were added, and reaction was left to stir overnight at room temperature. The reaction mixture was diluted with DCM, washed with Brine, and then with saturated ammonium chloride solution twice. The organic layer was separated and dried, then the organic solvent was evaporated to dryness and the product was dried under high vacuum. The product was obtained as a white solid in quantitative yield (1.85 grams).

¹H NMR (400 MHz, Chloroform-d) δ 5.98-5.84 (m, 1H, CH₂═CH—CH₂—O—), 5.38-5.06 (m, 83H, —O—CH(CH₃)—CO—, PLGA backbone and CH₂═CH—CH₂—O—), 4.97-4.51 (m, 100H, —O—CH₂—CO—, PLGA backbone), 4.01 (dt, J=5.6, 1.5 Hz, 2H, CH₂═CH—CH₂—O—), 3.88-3.40 (m, 181H, —O—CH₂—CH₂—O—, PEG backbone), 2.06 (s, 3H, —O—CO—CH₃), 1.77-1.35 (m, 250H, —O—CH(CH₃)—CO—, PLGA backbone). SEC (DMF+25 mM NH₄Ac, PEG standards calibration): Mn=4.3 kDa, Ð=2.0; see, FIG. 28B.

Synthesis of AcO-PLGA₁₂ₖ-PEG₂ₖ-glycerol (polymer 13): Polymer 12 (1.80 gram, 1 equivalents), 1-thioglycerol (430 μL, 30 equivalents) and DMPA (13 milligrams, 0.3 equivalents; 1 mol % with respect to the thiol) were dissolved in DMF (3 mL per 1 gram of hybrid). The solution was purged with nitrogen for 30 minutes and then stirred under UV light (365 nm) for 2 hours. Then, the reaction mixture was dissolved in minimal amount of DCM, and product was precipitated using slow addition of ether (150 mL). The obtained solid was filtered, washed with ether, and finally dried under high vacuum. The product was obtained as a white solid in quantitative yield (1.80 grams).

¹H NMR (400 MHz, Chloroform-d) δ 5.39-5.05 (m, 83H, —O—CH(CH₃)—CO—, PLGA backbone), 4.98-4.51 (m, 100H, —O—CH₂—CO—, PLGA backbone), 3.88-3.40 (m, 186H, —O—CH₂—CH₂—O—, PEG backbone and glycol), 2.73-2.52 (m, 4H, —CH₂—S—CH₂—), 2.13 (s, 3H, —O—CO—CH₃), 1.93-1.76 (m, 2H, —O—CH₂—CH₂—CH₂—S—), 1.77-1.35 (m, 250H, —O—CH(CH₃)—CO—, PLGA backbone). SEC (DMF+25 mM NH₄Ac, PEG standards calibration): Mn=5.0 kDa, Ð=1.8; see, FIG. 28B.

Synthesis of PLGA-PEG-Glycerol-(SO₃)₂ (Polymer 14): Polymer 13 (980 milligrams, 1 equivalents) was dissolved in dry DMF (5 mL per 1 gram) and SO₃-DMF complex (270 milligrams, 20 equivalents) was added to the solution. Then the reaction mixture was heated to 65° C. for 2 hours. The solvent was evaporated, and the mixture was quenched by the addition of 5 mL aqueous NaHCO₃ and further diluted with 50 mL water. The aqueous mixture was left in the fridge overnight, and the polymer was precipitated and separated from the solution using centrifuge. The white solid was then re-suspended in water, cooled, and centrifuged. This step was repeated twice more. The precipitate was finally re-suspended in water and lyophilized to obtain the final product in 83% yield (810 milligrams). It should be noted that after precipitation, lower MW polymers were separated, and Mn of PLGA block increased.

Due to interactions of the sulfonates with the column, this hybrid was not analyzed by SEC.

¹H NMR (400 MHz, Chloroform-d) δ 5.32-5.05 (m, 120H, —O—CH(CH₃)—CO—, PLGA backbone), 4.96-4.53 (m, 137H, —O—CH₂—CO—, PLGA backbone), 3.86-3.41 (m, 186H, —O—CH₂—CH₂—O—, PEG backbone and glycol), 2.82-2.65 (m, 4H, —CH₂—S—CH₂—, partially falls under water), 2.12 (s, 3H, —O—CO—CH₃), 1.93-1.81 (m, 2H, —O—CH₂—CH₂—CH₂—S—), 1.77-1.34 (m, 360H, —O—CH(CH₃)—CO—, PLGA backbone).

Due to the intended interaction with the SELP molecule presented on the surface of the cancer cells and the activated endothelial cells, the presence of —SO₃ groups on the surface of the nanoparticles was assessed.

To ascertain the elemental makeup of the exemplary PLGA-PEG-Glycerol-(SO₃)₂ (Polymer 14) NPs and to confirm the presence of sulfate groups on their surface, a surface-specific quantitative XPS analysis was conducted.

The XPS Survey (FIG. 33A) reveals characteristic peaks for carbon, nitrogen, oxygen, and sulfur, indicating the presence of these elements on the sample surface. Additional peaks may suggest the presence of sodium and chlorine. At higher binding energies (above 700 eV), peaks for metals or other less common elements are found. The sulfur peak around 160-170 eV suggests sulfonate groups, which are relevant for surface interactions that target SELP.

To accurately pinpoint the peak positions of each element, the C1s (C—C) bond peak was initially located at 284.86 eV, aligning closely with the theoretical expectation of 284.8 eV (FIG. 33B). Consequently, no adjustments were needed to align the observed results with theoretical values.

Table 5 below summarizes the peaks of the elements found during the XPS analysis including the position, the Full Width at Half Maximum, peak Area and atomic percentage of the element relative to the whole sample:

TABLE 5

| Name | Peak BE | FWHM ev | Area (P) CPS. ev | Atomic % |
|---|---|---|---|---|
| C 1s | 284.83 | 4.51 | 1837755.86 | 58.01 |
| O 1s | 531.61 | 4.25 | 1992495.93 | 25.17 |
| N 1s | 399.33 | 4.12 | 262186.43 | 5.25 |
| Na 1s | 1070.98 | 4.16 | 576570.27 | 3.30 |
| F 1S | 684.79 | 5.39 | 290079.11 | 2.86 |
| Ca 2p | 347.31 | 6.21 | 280741.92 | 1.53 |
| S 2p | 168.48 | 4.05 | 63611.04 | 1.01 |
| B 1s | 189.10 | 4.45 | 10692.49 | 0.86 |
| Mg 2s | 89.02 | 4.60 | 21693.56 | 0.82 |
| Cl 2p | 198.50 | 5.05 | 47583.41 | 0.53 |
| P 2p | 133.03 | 3.73 | 18293.84 | 0.40 |
| P 2s | 191.40 | 3.75 | 5861.99 | 0.16 |
| Zn 2p | 1020.99 | 4.79 | 58098.41 | 0.10 |

FIG. 33C presents the high-resolution XPS spectra for the sulfate group peak located at 168.48 eV. It resolves the binding energies of the 2p3/2 and 2p1/2 orbitals, confirming the presence of sulfate functional groups on the surface of the PLGA-PEG-Glycerol-(SO₃)₂ nanoparticles. In FIG. 33D, the depth profile analysis of the S2p peak indicates a consistent peak shape across different sputtering times, each layer representing 60-second intervals of ion exposure.

Altogether, these data demonstrate that no significant variation in the sulfate content or the sulfur atomic percentage relative to carbon occurs within the top 30 nm of the nanoparticle surface, which indicates that the S-content is homogenously distributed in the surface of PLGA-PEG-Glycerol-(SO₃)₂ 20%.

An additional exemplary sulfated polymer, PLGA-PEG-Glycerol-(SO₃)₄ amphiphilic hybrid, was synthesized as described in FIG. 28C, using Click chemistry. The chemical structure was verified by ¹H-NMR (data not shown).

### Example 9

The Effect of SELP-targeting Moieties on NPs Internalization into 3D spheroids Following the addition of the hydrophilic sulfate moieties, the physicochemical properties were examined alongside their internalization rate into SELP-expressing 3D spheroids.

The novel sulfated PLGA-PEG polymers were incorporated in the formulation, replacing the non-modified PLGA-PEG. The physicochemical properties (size, conductivity, and polydispersity) of the SELP-targeted Cy5-labeled NPs were compared to that of the non-targeted NPs (PLGA-PEG-Cy5).

The results are presented in FIGS. 20A-C and 20D-F and are summarized in Table 6 below. The table shows the average (mean±SD) of three independent measurements.

TABLE 6

| | Size by DLS | Z potential | Polydispersity |
|---|---|---|---|
| Sample | [nm] | [mV] | (PD) index [Ð] |
| PLGA-PEG-Cy5 | 96 ± 13 | −1.6 ± 0.9 | 0.12 |
| PLGA-PEG-SO₃- | 130 ± 2 | −3.7 ± 2.4 | 0.08 |
| Cy5 | | | |
| PLGA-PEG- | 113 ± 5 | −3.4 ± 2.7 | 0.11 |
| Glycerol-(SO₃)₂-Cy5 | | | |

As can be seen from Table 6, all NPs had an approximate 100 nm diameter size, a narrow size distribution, and a nearly neutral zeta potential. FIGS. 20A-C and 20D-F present TEM images showing that the NPs has a spherical shape.

The internalization rate into SELP-expressing 3D spheroids was then evaluated by treating human and murine spheroids with solutions containing 1 μM Cy5-equivalent NPs.

As can be seen in FIGS. 20G and 20H, the exemplary PLGA-PEG-Glycerol-(SO₃)₂ (Polymer 8) NPs internalize into the spheroids to a significantly higher extent than the other groups already after 8 hours. During the first 4-6 hours, the NPs slowly began to accumulate in the spheroids at a similar rate, but after 8 hours, PLGA-PEG-Glycerol-(SO₃)₂ NPs demonstrated an enhanced internalization rate which continued to grow during the experiment intervals and the final Cy-5 signal was considerably higher in both cell lines. The exemplary PLGA-PEG-SO₃, which contained one sulfonate group, internalized into the spheroids more than the non-targeted NPs did.

Overall, an increase in the internalization of targeted-NPs into 3D spheroids was observed in the presence of the exemplary sulfated polymer PLGA-PEG-Glycerol-(SO₃)₂.

Next, the spheroids were treated with SELP inhibitor (SELPi) 1 hour before the addition of the NPs, and the results are presented in FIGS. 21A-D.

The internalization of PLGA-PEG-Glycerol-(SO₃)₂ (Polymer 8) and PLGA-PEG-SO₃ NPs was reduced by 5-fold and 1.5-fold in D4M.3A spheroids and by 1.5-fold and 1.3-fold in WM115 spheroids, after treatments with 10 μM SELPi, accordingly, whereas the internalization of the non-targeted NPs was not affected.

Taken together, these data suggest a SELP-mediated internalization mechanism of the exemplary sulfated PLGA-PEG NPs.

The binding abilities of the PLGA-PEG-Glycerol-(SO₃)₂ polymer 14 were also validated. Murine EMT6 spheroids were incubated with 1 μM Cy5-equivalent SELP-targeted NPs with or without 10 μM SELPi for 20 hours.

As can be seen in FIGS. 34A-B, Similarly to the previous observation using melanoma models, the internalization of SELP-targeted NPs was reduced by 2-fold in the presence of SELPi, indicating SELP-mediated internalization mechanism of the optimized sulfated PLGA-PEG-Glycerol-(SO₃)₂ (Polymer 14) NPs.

### Example 10

### SELP Binding Abilities and Tumor Accumulation of SELP-Targeted NPs

The tumor accumulation of Cy5-labeled targeted NPs, non-targeted NPs, or DBF-Cy5 was assessed in C57BL/6 mice that were inoculated SC with D4M.3A cells. Once the tumor size reached about 200 mm³, the mice were IV administered with the treatments, and the Cy5 signal from the tumor was followed for 2 hour.

The results are presented in FIGS. 22A-C. As can be seen, the Cy5 signal of PLGA-PEG-Glycerol-(SO₃)₂ (Polymer 8) was 5-times higher than the Cy5 signal from DBF-Cy5 and PLGA-PEG NPs and 2.6-times higher than PLGA-PEG-SO₃ NPs signal, which corresponded to the enhanced internalization observed in the 3D spheroids.

FIGS. 22B-C indicate that the sulfate or sulfonate modification did not alter the NPs distribution to essential organs and resembled the distribution of non-targeted NPs with negligible accumulation in the liver after 24 hours, which is essential to prevent adverse events.

The tumor accumulation of Cy5-labelled PLGA-PEG-Glycerol-(SO₃)₂ (Polymer 14) NPs was then compared to non-targeted PLGA-PEG-Cy5 NPs. BALB/C mice were intramammary fat pad inoculated with EMT6 cells and allowed to establish tumors for 10 days. The mice were IV-treated with 20 μM Cy5-equivalent NPs and the Cy5 signal was monitored for 24 hours, and the results are presented in FIGS. 34C-D. As can be seen, 3 and 24 hours post-injection, the Cy5 signal from the harvested tumors was significantly higher for the PLGA-PEG-Glycerol-(SO₃)₂-Cy5 NPs than for the PLGA-PEG-Cy5 NPs (1.7 vs. 1.1 scaled counts s⁻¹ after 3 hours and 2.4 vs. 1.1 scaled counts s⁻¹ after 24 hours, respectively). It should be noted that 24 hours post-administration the high Cy5 signal from the tumors indicates the retention of PLGA-PEG-Glycerol-(SO₃)₂-Cy5 NPs inside the tumors.

In-addition, 24 hours post administration, the distribution to essential organs was assessed (see, FIGS. 34E-F), showing highest accumulation in the liver, as the main elimination pathway, followed by the tumor for PLGA-PEG-Glycerol-(SO₃)₂-Cy5 NPs and the spleen for the PLGA-PEG-Cy5 NPs.

Furthermore, the NPs brain accumulation was assessed on BALB/C female mice, 12 days after intracranial injection of EMT6 cells or D4M.3A cells, as schematically depicted in FIG. 3G. As FIG. 34H shows, for EMT6 cells, 6 hours post-administration, the NPs accumulated in the brain 3-times higher than the free drug, which suggest that the SELP-targeted NPs allow enhanced tumor accumulation in SELP-expressing BC orthotopic model and brain metastases, consequently mediating improved drug accumulation in these compartments. Improved accumulation was also observed in the D4M.3A cells-induced model. The drugs were DBF for the D4M.3A cells and talazoparib for the EMT6 cells.

These data show the internalization of the exemplary drug-loaded PLGA-PEG-Glycerol-(SO₃)₂ (Polymer 14) NPs into 3D spheroids is SELP-mediated. These data further suggest that these NPs accumulated in orthotopic models of BRCA-mutated BC.

### Example 11

### Antitumor Activity of Drug-Loaded SELP-Targeted NPs

The in vivo activity of the novel NPs was then studied.

Physicochemical characterization (drug loading content (DLC), drug loading efficiency (DLE), conductivity, and dispersity) of the SELP-targeted NPs loaded with DBF and TRM are presented in FIGS. 23A-C and are summarized in Table 7 below.

TABLE 7

| | | Size | | | | | | |
|---|---|---|---|---|---|---|---|---|
| | Size by | by | DLC | DLC | DLE | DLE | Z | Polydispersity |
| | DLS | TEM | DBF | TRM | DBF | TRM | potential | (PD) index |
| Sample | [nm] | [nm] | [wt %] | [wt %] | [%] | [%] | [mV] | [Ð] |
| PLGA-PEG- | 90 ± 5 | 64 ± 14 | 6.8 ± 0.3 | 0.7 ± 0.1 | 35 ± 3 | 33 ± 8 | −1.1 ± 1.6 | 0.15 ± 0.06 |
| SO₃ | | | | | | | | |
| PLGA-PEG- | 100 ± 7 | 53 ± 13 | 6.2 ± 0.2 | 0.7 ± 0.1 | 28 ± 5 | 28 ± 5 | −0.8 ± 0.5 | 0.16 ± 0.04 |
| Glycerol- | | | | | | | | |
| (SO₃)₂ | | | | | | | | |

Table 7 shows that DLE [%] and DLC [wt %] were similar to those observed for the non-targeted NPs. Additionally, the NPs maintained a low dispersity and a constant hydrodynamic diameter for 72 hours incubation in PBS. The zeta potential was almost neutral owing to the high content of ester terminated PLGA in the NPs matrix. As can be seen in FIG. 23D, the release profile of DBF and TRM also resembled that of the non-targeted NPs (FIG. 3D). TRM release half-life was 17 hours for both formulations, and DBF release half-life was 7 hours for PLGA-PEG-Glycerol-(SO₃)₂ (Polymer 8) NPs and 14 hours for PLGA-PEG-SO₃ NPs.

Without being bound to any particular theory, it is assumed that, due to the hydrophobicity and higher loading of DBF into the NPs, its encapsulation efficacy and release profile are more affected by the addition of hydrophilic sulfate moieties.

Furthermore, as can be seen in FIGS. 23E-F, the NPs did not affect the viability of NIH/3T3 murine fibroblast cells and did not cause hemolysis to red blood cells, indicating that the NPs were biocompatible and safe for systemic administration.

The following data demonstrate the superior antitumor activity and prolonged survival of targeted PLGA-PEG-Glycerol-(SO₃)₂ (Polymer 8) NPs in comparison to non-targeted PLGA-PEG NPs and free drugs.

The anti-tumor activity of the targeted NPs was demonstrated while examining SELP-expression on both the activated endothelial cells at the tumor site and the tumor cells.

For this purpose, C57BL/6 mice were SC inoculated with D4M.3A cells and were allowed to establish tumors until day 10, once the average tumor volume was 50 mm³. The mice were then randomized into groups containing 5-10 mice per group and were IV administered with the combination of 1 mg/kg DBF and 0.1 mg/kg TRM as free drugs, and targeted or non-targeted drug-loaded NPs. The previously-administered low dose was selected since there was no survival benefit, or increased tumor inhibition, to the high dose compared to the low dose of NPs, as seen in FIGS. 16B-G. The mice were treated QOD until the first group reached the endpoint of tumor volume above 1000 mm³ (18 doses). A schematic presentation of the study protocol and timeline is presented in FIG. 24A.

The results are presented in FIGS. 24B-F, and as can be seen, the combination of free DBF and TRM retained a small tumor volume (below 250 mm³) until day 31, while with the non-targeted and PLGA-PEG-SO₃ NPs, it was retained until day 39. Surprisingly, 11 days after treatment withdrawal (day 57), the average tumor size of mice treated with the exemplary SELP-targeted NPs, PLGA-PEG-Glycerol-(SO₃)₂ (Polymer 8) NPs, was still below 250 mm³ (FIG. 24B) and 7/10 mice had tumor size below 150 mm³ (FIG. 24E). A survival benefit was achieved, with a median survival of 80 days for mice treated with the PLGA-PEG-Glycerol-(SO₃)₂ (Polymer 8) NPs in comparison to 57-58 days for the other treated groups and 29 days for the PBS group (FIG. 24G).

A safety evaluation was made by monitoring the bodyweight change of the treated mice. As can be seen in the body-weight change graph in FIG. 24F, neither group suffered from abnormal weight changes that could indicate induced toxicity.

Immunostaining for cell proliferation (Ki-67) and apoptosis (cleaved caspase-3), which were performed after the ninth treatment are presented in FIGS. 25A-D. A notably decreased Ki-67 staining in PLGA-PEG-Glycerol-(SO₃)₂-treated group is observed compared to the other groups. Similarly, increased staining for cleaved caspase-3, supported the better anti-tumor efficacy obtained by the exemplary SELP-targeted PLGA-PEG-Glycerol-(SO₃)₂ (Polymer 8) NPs.

In addition, following the 9^{th} treatment, blood was drawn to perform a blood biochemistry analysis. The data does not indicate hepatic or renal toxicities took place following administration of the free drugs or the drug-loaded NPs (data not shown).

These data demonstrate that co-encapsulation of DBF and TRM in NPs, and especially in SELP-targeting NPs, results in reduced tumor growth and prolonged survival of melanoma-bearing mice, while reducing the concentration of DBF by 30 times and the concentration of TRM by 3 times compared to the concentration of the free drugs.

Additional data demonstrate a superior activity of SELP-targeting NPs co-encapsulating DBF and TRM compared to a combination of SELP-targeting NPs encapsulation DBF and SELP-targeting NPs encapsulation TRM in melanoma spheroids; IC50 values of SELP-targeting NPs encapsulation DBF, of a combination of SELP-targeting NPs encapsulation DBF and SELP-targeting NPs encapsulation TRM, and of SELP-targeting NPs co-encapsulating DBF and TRM in each particle were 1.1, 9.6 and 14.9 nM, respectively.

### Example 12

### Effect of Drug-Loaded SELP-Targeted NPs on BRCA-Mutated Cells

The encapsulation of additional drugs (e.g., antitumor agents) by the exemplary SELP-targeted NPs was examined. Herein, BRCA-mutated cells were targeted.

The effect of the exemplary sulfated PLGA-PEG NPs loaded with talazoparib and the PD-L1i was examined on BRCA-mutated BC cells. Four different TNBC cell lines were treated with a serial dilution of talazoparib, including BRCA-mutated murine EMT6 and human MDA-MB-436 cells, and murine BRCA wild-type 4T1 and MDA-MB-231 cells.

As can be seen in FIGS. 29A-D, talazoparib was the most potent among the tested PARPi with preferred cytotoxic activity in BRCA-mutated cells compared to wild type cells with IC₅₀ values of 10 nM and 2 nM for EMT6 and MDA-MB-436 cells, compared to 58 nM and 411 nM for 4T1 and MDA-MB-231 cells, respectively. These data suggest that talazoparib induces a synthetic lethality in BRCA-mutated BC cells, characterized by deficient homologous recombination [A. Huang (2020), supra].

Without being bound to any particular theory, it is possible that the activity was more cytotoxic in the BRCA-mutated cell lines due to their deficient homologous recombination and consequently synthetic lethality displayed by the PARPi [A. Turk, K. B. Wisinski, PARP Inhibition in BRCA-Mutant Breast Cancer. Cancer 124, 2498-2498 (2018); A. Huang, L. A. Garraway, A. Ashworth, B. Weber, Synthetic lethality as an engine for cancer drug target discovery. Nature Reviews Drug Discovery 19, 23-38 (2020); S. Ferber et al., Co-targeting the tumor endothelium and P-selectin-expressing glioblastoma cells leads to a remarkable therapeutic outcome. Elife 6 (2017)].

Subsequently, the cytotoxic activity of talazoparib was demonstrated on 3D spheroids of mCherry-labelled EMT6 cells, and the results are presented in FIGS. 29E-F. As can be seen, a 70% inhibition of the spheroids' sprouting was observed following treatments with 1 μM talazoparib compared to the control. Indeed, talazoparib showed potent anti-tumoral effects against mCherry-labeled EMT6 3D spheroids; increasing concentrations of talazoparib reduced the ability of mCherry-labeled EMT6 3D spheroids to sprout in Matrigel®. Therefore, talazoparib was the therapeutic agent selected to be entrapped in the exemplary PLGA-PEG NP.

Then, it was examined whether treatment with PARPi upregulated PD-L1 expression, as previously reported in other cancer types [S. Jiao (2017), supra]. As can be seen in FIG. 30A, treatments with talazoparib induced PD-L1 expression in EMT6 cells in a dose-dependent manner. The synergistic potential of an exemplary poly ADP ribose polymerase (PARP) inhibitor, talazoparib, and a PD-L1 inhibitor (PD-L1i) was demonstrated after showing increased levels of PD-L1 following 24-72 hour-treatments with 10 nM talazoparib (FIGS. 26A-B). The time-dependent increase in PD-L1 expression can be observed both at RNA level and protein expression.

dSTORM super-resolution imaging were also performed, and it showed an increased number of PD-L1 localizations and higher PD-L1 protein density following treatments with increasing concentrations of talazoparib (FIGS. 30B-C).

Altogether, these data suggest that treatments with the exemplary PARPi, talazoparib, can trigger tumor immunosuppression and consequently reduce the treatment efficiency. To meet this challenge, a combination treatment of talazoparib and the exemplary PD-L1i was tested in vitro.

For this purpose, BRCA-mutated EMT6 cells were incubated with 10 μM SM56 for 24-72 hours. As can be seen in FIG. 30D, the exemplary PD-L1i SM56 caused a reduction in PD-L1 expression in a time-dependent manner. Then, the EMT6 cells were treated with the combination of 10 nM talazoparib and 10 μM PD-L1i to assess their ability to abrogate PD-L1 upregulation. As seen in FIG. 30E, treatment with talazoparib alone upregulated PD-L1 expression by 3-folds, while treatments with the exemplary PD-L1i alone reduced PD-L1 expression by about 40% compared to untreated cells. The addition of the exemplary PD-L1i (SM56) to talazoparib reduced PD-L1 expression from 3 to 1.5 folds.

The anti-proliferative effect was also assessed in mCherry-labelled EMT6 3D spheroids. The spheroids were established in a U-well shaped plate for 72 hours, then treated with 1 μM talazoparib, 10 μM PD-L1i separately or combined, and lastly co-cultured with activated splenocytes. The activated splenocytes alone inhibited the spheroids growth, but the most significant inhibition was achieved for the combined treatment (FIGS. 30F-G). These data confirm the rational for the combination of the compounds for the treatment.

SELP expression was evaluated in primary and brain metastases of EMT6 tumors, as its expression in BC human samples has been observed (not shown). The results, presented in FIGS. 31A-C, show that both primary and brain metastases express high levels of SELP. Additionally, SELP was also found to be expressed on EMT6 3D spheroids, which together verify the feasibility of SELP targeting in this BC model.

Consequently, an exemplary PLGA-PEG-Glycerol-(SO₃)₂ NPs (Polymer 14) were used to co-encapsulate the exemplary PARP inhibitor, talazoparib, and/or the small molecule PD-L1 inhibitor (PD-L1i) as described in Acúrcio et al. Journal for ImmunoTherapy of Cancer, 10(7), e004695 (2022) and in WO 2022/175955. Exemplary single drug-loaded NPs and dual drug-loaded NPs were successfully obtained, as can be seen in FIG. 26C. TEM images indicated a homogeneous and spherical morphology with an average diameter of 73 nm.

Physicochemical properties (drug loading content (DLC), drug loading efficiency (DLE), conductivity, and dispersity) of the obtained NPs are summarized in Table 8 below.

TABLE 8

| | | | DLC | | Z- | |
|---|---|---|---|---|---|---|
| | Size by | Size by | talazoparib | DLC PD-L1i | potential | Polydispersity |
| Sample | DLS [nm] | TEM [nm] | [wt %] | [wt %] | [mV] | index [Ð] |
| Unloaded | 121.1 ± 5.6 | 69.6 ± 6.2 | — | — | −0.20 | 0.10 |
| (blank) NPs | | | | | | |
| Talazoparib | 122.3 ± 6.6 | 74.1 ± 12.9 | 18 | — | −0.12 | 0.13 |
| (Tal) NPs | | | | | | |
| PD-L1i NPs | 131.83 ± 8.3 | 77.0 ± 13.7 | — | 10.5 | −0.30 | 0.13 |
| Talazoparib + | 130.1 ± 13.6 | 71.3 ± 9.1 | 3.5 | 12.5 | −0.20 | 0.18 |
| PD-L1i | | | | | | |
| (Tal- PD- | | | | | | |
| L1i NPs) | | | | | | |

The DLC for talazoparib-loaded NPs (also denoted herein as “Tal NPs”) was 18 wt %, and for PD-L1i NPs it was 10.5% wt %. The DLC for talazoparib and PD-L1i co-encapsulation, at a 1:4 ratio, was 3.5 wt % and 12.5 wt % respectively. The NPs' average hydrodynamic diameter was 125 nm, with a narrow size distribution (PDI=0.13), and a nearly neutral zeta potential.

Table 8 and FIG. 26C show that the NPs are compatible for the encapsulation of talazoparib and PD-L1i, separately or combined.

The release profile showed that the exemplary combined talazoparib and SM56-loaded NPs had a slower initial burst release of 40% after the first 3 hours compare to the single-loaded NPs, which was followed by a more sustained drug release up to 48 hours (FIG. 32A). Furthermore, The NPs were stable in PBS for 48 hours (FIG. 32B). In vitro, talazoparib-loaded NPs demonstrated similar anti-proliferative capabilities as free talazoparib with IC₅₀ values of 20 nM and 8 nM, respectively (FIG. 32C). Importantly, blank NPs (unloaded NPs) did not affect the EMT6 proliferation nor caused red blood cell lysis up to 5 mg/mL, implying their safety for systemic administration (FIGS. 32E-F).

BALB/C female mice were inoculated in the intramammary fat pad with EMT6 cells and were allowed to establish tumors until the average tumor volume was 50 mm³ (day 15). Then, the mice were randomized into groups containing 6-9 mice per group and were IV administered with the combination of 3 mg/kg SM56 and 0.3 mg/kg talazoparib as free compounds, and SELP-targeted or non-targeted drug-loaded NPs, three times a week. The combination of free talazoparib and the exemplary PD-L1i did not significantly reduce the tumor growth compared to the control groups (PBS, vehicle and PLGA-PEG-Gly-(SO₃)₂ carrier), but the SELP-targeted NPs reduced substantially the tumor growth compared to the control groups and free compounds (FIG. 35A). Notably, neither group suffered from abnormal weight changes that could indicate induced toxicity (FIG. 35B).

These results show that the exemplary sulfated PLGA-PEG NPs according to some embodiments of the present invention, PLGA-PEG-Glycerol-SO₃ NPs, when loaded with drugs, allows superior anti-tumor activity compared to non-targeted PLGA-PEG NPs and to the non-encapsulated (free) drugs.

An overall suggested mechanism for the anti-tumor activity of sulfated PLGA-PEG NPs is presented in FIG. 36, and includes the following steps:
1) The ligand-targeted NPs selectively bind to P-Selectin, present in the surface of the BRCA mutated BC cell lines;
2) The nanoparticles internalize to the cellular cytosol;
3) Drug release and PARP inhibition;
4) Upregulation of PD-Li;
5) PD-L1i small molecule abrogates PD-L1 upregulation; and
6) T-cell expansion and antitumoral effect.

PLGA-PEG-Glycerol-(SO₃)₂ NP-entrapped drug combinations are also tested in other BRCA-mutated cancer types, such as pancreatic and ovarian cancers.

PLGA-PEG-Glycerol-(SO₃)₂ NP-entrapped drug combinations of PARPi, such as talazoparib, and other anti-cancer drugs such as berzosertib (ATR inhibitor) or topotecan (topoisomerase inhibitor), as described herein, are also tested.

### Example 13

### Concluding Insights

Based on the feasibility of SELP expression, SELP-targeted PLGA NPs were synthesized. Two polymers that contained sulfate moieties conjugated to PLGA-PEG polymer were synthesized and their physicochemical characterization, internalization, and anti-tumor efficacy were evaluated.

The sulfate-modified NPs internalized into 3D spheroids to a higher extent than non-modified NPs. Specifically, the highest internalization was achieved by the exemplary PLGA-PEG-Glycerol-(SO₃)₂ NPs, followed by the exemplary PLGA-PEG-SO₃ NPs, which contained two and one sulfate moieties on a single PLGA-PEG polymer, respectively.

The PLGA-PEG NPs self-assemble to form a hydrophobic core, which enabled the encapsulation of the two exemplary hydrophobic drugs, DBF and TRM. PEG was used as a spacer for the sulfate moieties, so as to render the hydrophobic core intact. The consequent drug release profile remained unaffected by the presence of these hydrophilic sulfate moieties.

To evaluate the therapeutic advantage of the targeted NPs, their anti-tumor efficacy was compared to non-targeted NPs and free drugs. Treatments with exemplary DBF- and TRM-loaded PLGA-PEG-Glycerol-(SO₃)₂ NPs resulted in inhibition of tumor growth and prolonged survival. Moreover, the exemplary PLGA-PEG-Glycerol-(SO₃)₂ NPs substantially inhibited ki67 and increased the levels of cleaved caspase-3 in tumors 20 hours post-final dose.

In addition, an in vivo dose acceleration experiment showed that daily treatments with 30 mg/kg DBF reduced pERK in tumors for up to 18 hours post-dose administration. Specifically, 6 hours post-final dose, 89% inhibition of pERK, and 28% reduction in ki67 were observed [King et al. PLoS One 8, e67583 (2013)]. The exemplary PLGA-PEG-Glycerol-(SO₃)₂ NPs modified the pharmacodynamic markers longer and to a higher extent in comparison to other groups. It was assumed that by actively targeting the NPs to the tumor site, the drug concentrations within the tumor is increased, and consequently, produces a superior anti-tumor effect.

The schematic illustration of the overall process for the preparation and administration of the exemplary NPs is presented in FIG. 27.

The option to co-deliver a synergistic ratio of two therapeutically active agents, as demonstrated herein for DBF and TRM, in a P-selectin-targeted nanocarrier has been validated herein. As a result, DBF concentrations were reduced by 30 times and TRM concentration by 3 times, while inhibiting tumor growth and prolonging the survival of mice bearing melanoma tumors compared to free drugs and non-targeted NPs.

Then, an exemplary PARPi/PD-L1i-loaded sulfated PLGA-PEG NPs was developed to treat BRCA-mutated BC.

The anti-proliferative effect of several PARPi was assessed and talazoparib was selected, as it was shown to be the most potent. As a side effect of talazoparib, an increased expression of PD-L1 was observed following treatments with Talazoparib on EMT6 murine BRCA-mutated BC cell line.

The expression of PD-L1 was assessed when using increasing concentrations of talazoparib in EMT6 cells both by FACS and super resolution microscopy, dSTORM which allowed observing this upregulation at the single molecule level. Also, both at protein and RNA levels increasing expression of PD-L1 while increasing the exposure time of the EMT6 cells to the drug was found. This suggests that combining anti-PD-1/PD-L1 therapies with PARPi in BRCA-mutated would allow to offset the tumor immunosuppression associated to the PARPi therapy and obtain a synergistic anti-cancer effect.

The therapeutic potential of a PD-L1i small-molecule was therefore assessed, in BRCA-mutated TNBC in vitro model. First, the ability of an exemplary PD-L1i small-molecule to bind the membranal PD-L1 of EMT6 cells and reduce the FACS signal below the basal levels was observed. Moreover, the PD-L ii was able to abrogate the PD-L1 upregulation enhanced by PARPi therapy when the EMT6 cells were simultaneously treated with talazoparib and the PD-L1i. Also, the PD-L1i inhibited EMT6 proliferation when the cells were co-cultured with activated splenocytes.

In addition, the exemplary PD-L1i limited mCherry-labeled EMT6 3D spheroids proliferation when they were in co-culture with activated splenocytes. This anti-tumor effect was enhanced even more when the small-molecule was combined with talazoparib, suggesting synergism between both compounds. This synergism was observed when mCherry-labeled EMT6 spheroids were co-cultured with activated splenocytes. The combined treatment of PARPi with PD-L1i limited the 3D BC spheroid growth to a higher extent compared to the single treatments.

Thus, the co-encapsulation of an exemplary PD-L1i, as described in WO 2022/175955 and the exemplary PARPi, talazoparib, was performed.

These studies further demonstrate that sulfonation of the nanocarriers allows SELP targeting. Therefore, three more PLGA-PEG ligand-targeted NPs, with 1, 2 or 4 sulfate end groups, were synthesized to enhance the anti-tumoral effect of the proposed therapy to treat BRCA-mutated BC.

The sulfonated PLGA-PEG NPs kept the physicochemical properties of the non-targeted in terms of size and polydispersity although they were slightly more negatively charged. Among the synthesized candidates, the NPs with two sulfate end groups, the PLGA-PEG-Gly-(SO₃)₂ internalized faster and to a higher extent than the other sulfonated NPs and especially compared to the non-targeted PLGA-PEG NPs in the EMT6 3D spheroids. This nanocarrier was shown to be stable under physiological conditions and capable to co-encapsulate the PD-L1i and talazoparib, biocompatible and suitable for being administered systemically. In vivo experiments further supported the accumulation of the sulfonated NPs more than the non-targeted NPs in the BRCA-mutated BC orthotopic mice model.

Thus, a SELP-targeted nanomedicine for BRCA-mutated BC was developed, combining PARPi therapy and immunotherapy, and displaying a more powerful therapeutic activity than the non-targeted nanomedicine and specifically more than the co-administration of the free drugs.

The targeted nanoparticles as described herein demonstrate the applicability thereof as delivery vehicles for a variety of relevant drugs.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A composition comprising a plurality of particles, wherein in at least a portion of said particles, each particle comprises a polymeric matrix having associated therewith at least one therapeutically active agent usable in treating a medical condition associated with an overexpression of P-selectin in a subject in need thereof,
wherein in at least a portion of said particles which comprise said polymeric matrix, said polymeric matrix has attached to a surface thereof a P-selectin selective targeting moiety represented by Formula I: 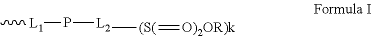
or a pharmaceutically acceptable salt thereof,
wherein:
R is hydrogen or alkyl;
the curved line represents an attachment point to the polymeric matrix;
P is an amphiphilic polymeric or oligomeric moiety;
L₁ and L₂ are each independently a linking moiety or absent; and
k is an integer ranging from 1 to 10, or from 1 to 6, or from 1 to 3,
wherein when k is greater than 1, L₂ is or comprises a branching unit,
and wherein an average molecular weight of said polymeric moiety ranges from about 100 to about 10,000, or from about 500 to about 5,000, or from about 1,000 to about 5,000, or from about 1,000 to about 3,000 grams/mol.

2. The composition of claim 1, wherein P is or comprises a poly(alkylene glycol) moiety.

3. The composition of claim 1, wherein L₁ and L₂ are each independently selected from an alkyl, an aminoalkyl, a hydroxyalkyl, a thioalkyl, an ether, a thioether, —O—, —S—, an amine, —C(═O)—, —C(═S)—, an amide, a carbamate, a carboxylate, a thiocarboxylate, a thiocarbamate, a thioamide, sulfonate, sulfoxide, phosphonate, sulfonamide, urea, thiourea, hydrazine, hydrazide, a hydrocarbon substituted or interrupted by any of the foregoing, and any combination thereof.

4. The composition of claim 1, wherein L₁ is or comprises an amine or an aminoalkyl.

5. The composition of claim 1, wherein L₂ is or comprises an amine or an aminoalkyl.

6. The composition of claim 5, wherein k is 1.

7. The composition of claim 1, wherein L₂ is or comprises a hydrocarbon interrupted by one or more of —O—, —S—, an amine, —C(═O)—, —C(═S)—, an amide, a carbamate, a carboxylate, a thiocarboxylate, a thiocarbamate, and a thioamide.

8. The composition of claim 1, wherein said targeting moiety is represented by: 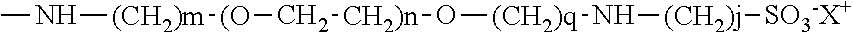
wherein:
n is an integer of at least 10, or at least 20;
each of m, q and j is independently 0, 1, 2, 3 or 4; and
X⁺ is a monocation.

9. The composition of claim 1, wherein k is greater than 1, and L₂ is or comprises said branching unit.

10. The composition of claim 9, wherein said branching unit is derived from glycerol.

11. The composition of claim 10, wherein k is 2 and said targeting moiety is represented by: 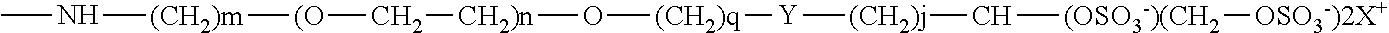
wherein:
n is an integer of at least 10, or at least 20;
each of m, q and j is independently 0, 1, 2, 3 or 4;
X⁺ is a monocation; and
Y is selected from —O—, —S—, an amine, —C(═O)—, —C(═S)—, an amide, a heteroaryl, a heteroalicyclic, a carbamate, a carboxylate, a thiocarboxylate, a thiocarbamate, and a thioamide.

12. The composition of claim 1, wherein said medical condition is a SELP-expressing cancer.

13. The composition of claim 1, wherein said medical condition is selected from melanoma, a primary brain cancer, a colon cancer, a pancreatic cancer, a non-small cell lung cancer, an ovarian carcinoma, a head and neck squamous cell carcinoma, a breast cancer, a kidney cancer, a pediatric glioma metastases thereof, and inflammation.

14. The composition of claim 1, wherein said at least one therapeutically active agent is or comprises an agent selected from an agent that downregulates an activity of MEK and/or BRAF; an immune checkpoint inhibitor; an agent that interferes with an activity or expression of PD1 and/or PDL1; an agent that interferes with an interaction between PD1 and PDL1; a PARP inhibitor; and a topoisomerase 1 inhibitor.

15. The composition of claim 1, comprising at least two of said therapeutically active agents.

16. The composition of claim 15, wherein said at least two therapeutically active agents act in synergy in treating said medical condition.

17. The composition of claim 15, wherein in at least a portion of said particles, each particle comprises said at least two therapeutically active agents.

18. The composition of claim 15, wherein in at least a portion of said particles each particle comprises a first therapeutically active agent and in at least another portion of said particles each particle comprises a second therapeutically active agent, and wherein said first and second therapeutically active agents act in synergy.

19. The composition of claim 1, being a pharmaceutical composition that further comprises a pharmaceutical acceptable carrier.

20. A method of treating associated with an overexpression of P-selectin in a subject in need thereof, the method comprising administering to the subject the composition of claim 1.
